(19) United States
(12) Patent Application Publication
(10) Pub. No.: US 2005/0272089 A1
(43) Pub. Date: Dec. 8, 2005

(54) Critical genes and polypeptides of haemophilus influenzae and methods of use

(76) Inventors: John E. Mott, Kalamazoo, MI (US); Catherine M. Trepod, Portage, MI (US); Staffan Arvidson, Akersberga (SE)

Correspondence Address: MUETING, RAASCH & GEBHARDT, P.A., P.O. BOX 581415, MINNEAPOLIS, MN, 55458, US

(21) Appl. No.: 11/194,246

(22) Filed: Aug. 1, 2005

Related U.S. Application Data
(62) Division of application No. 10/274,586, filed on Oct. 21, 2002, now abandoned.
(60) Provisional application No. 60/345,438, filed on Oct. 19, 2001.

(51) Int. Cl. C12Q 1/68; G01N 33/554; G01N 33/569; C07H 21/04; C12N 1/20; C07K 14/195

(52) U.S. Cl. 435/6; 435/69.1; 435/252.3; 435/7.32; 530/350; 536/23.7

(57) ABSTRACT

The present invention provides methods of identifying agents that bind polypeptides critical for the survival of Haemophilus spp., preferably H. influenzae. The present invention also provides critical polypeptides and the polynucleotides encoding the critical polypeptides.

### CONTINUING APPLICATION DATA

This application claims the benefit of U.S. Provisional Application Ser. No. 60/345,438, filed Oct. 19, 2001, which is incorporated by reference herein.

### BACKGROUND

The increasing presence of antibiotic resistant bacteria in the clinical setting has renewed interest within the pharmaceutical industry for the development of new classes of antimicrobial agents. One strategy for the identification of potential new antibiotics is to develop molecular screens against novel antibacterial targets that are essential for the survival of the bacterium. Essential gene products traditionally have been identified through the isolation of conditional lethal mutants or the directed deletion of the target gene in the presence of a complementation vector. However these approaches are time consuming, laborious, and limited to bacteria with well defined genetic systems. The complete sequence analysis of many microbial genomes has led to the development of in vitro transposon mutagenesis as a more rapid method for identifying essential genes in bacteria. Transposon mutagenesis results in the disruption of the gene into which the transposon has inserted. Following recombination into the host chromosome, if the insertion allows the bacteria to survive and form colonies then it is unlikely that the gene is essential for bacterial viability under those conditions.

Haemophilus influenzae is a small, gram-negative, facultative anaerobic bacilli that frequently resides in the human upper respiratory tract. Infection by H. influenzae occurs most commonly in children, where it causes meningitis, sepsis, epiglotlitis, pneumonia, sinusitis, and where it is isolated in up to 25% of cases of otitis media. H. influenzae infection occurs in adults, typically those compromised by other conditions including diabetes and AIDS.

In the past, antibiotics were often effective to treat H. influenzae infections; however, antibiotic resistant strains have become prevalent. Thus, there continues to exist a need for new agents useful for treating bacterial infections, particularly those caused by antibiotic-resistant H. influenzae, and for methods of identifying such new agents. Such methods ideally would identify agents that are unrelated to existing antimicrobials and that target different aspects of H. influenzae pathogenesis in the host, compared to existing antimicrobials.

### SUMMARY OF THE INVENTION

The present invention provides isolated polynucleotides. A polynucleotide may include a nucleotide sequence of the coding sequence in SEQ ID NO:11, 16, 26, 31, 46, 64, 69, 74, 79, 84, 89, 99, 104, 109, 114, 124, 129, 145, 165, 170, 180, 205, 210, 215, 220, 232, 237, 242, 247, 252, 357, 150, 185, 200, 267, 389-443, nucleotides 936-2429 of SEQ ID NO:225, nucleotides 2443-3809 of SEQ ID NO:225, or the complements thereof. In another aspect, a polynucleotide may include a coding sequence encoding a critical polypeptide having structural similarity, for instance, at least about 95 percent structural similarity, with an amino acid sequence of SEQ ID NO: 282, 283, 285, 286, 289, 292, 293, 294, 295, 296, 297, 299, 300, 301, 302, 304, 305, 307, 308, 311, 312, 314, 315, 318, 321, 322, 323, 324, 327, 328, 329, 340, 341, 343, 345,325, 326, 444-498 or may include a coding sequence encoding an essential polypeptide having structural similarity, for instance, at least about 95 percent structural similarity, with an amino acid sequence selected from the group consisting of SEQ ID NO: 282, 283, 285, 286, 289, 293, 294, 295, 296, 297, 300, 301, 302, 304, 307, 311, 312, 314, 321, 322, 323, 324, 327, 328, 329, 340, 341, 345, 325, 326, and 444-498.

The present invention also provides isolated polypeptides. A polypeptide may include an amino acid sequence of SEQ ID NO:282, 283, 285, 286, 289, 292, 293, 294, 295, 296, 297, 299, 300, 301, 302, 304, 305, 307, 308, 311, 312, 314, 315, 318, 321, 322, 323, 324, 327, 328, 329, 340, 341, 343, 345, 325, 326, and 444-498. In another aspect, the polypeptide is a critical polypeptide, preferably, an essential polypeptide, may have an amino acid sequence having structural similarity, for instance, at least about 95 percent structural similarity, with an amino acid sequence of SEQ ID NO: 282, 283, 285, 286, 289, 292, 293, 294, 295, 296, 297, 299, 300, 301, 302, 304, 305, 307, 308, 311, 312, 314, 315, 318, 321, 322, 323, 324, 327, 328, 329, 340, 341, 343, 345, 325, 326, and 444-498.

Also provided by the present invention is a method for identifying an agent that binds a polypeptide. The method includes combining a polypeptide and an agent to form a mixture, and determining whether the agent binds the polypeptide. The polypeptide is encoded by a polynucleotide of the present invention. Determining whether the agent binds the polypeptide can include an assay, for instance, an enzyme assay, a binding assay, or a ligand binding assay.

The method may further include determining whether the agent decreases the growth rate of a microbe. Determining whether the agent decreases the growth rate includes combining a microbe with the agent, incubating the microbe and the agent under conditions suitable for growth of a microbe that is not combined with the agent, and determining the growth rate of the microbe combined with the agent. A decrease in growth rate compared to the microbe that is not combined with the agent indicates the agent decreases the growth rate of the microbe. Preferably the microbe is H. influenzae, and preferably, the microbe is in vitro or in vivo. The present invention includes an agent identified by the method.

In another aspect of such methods for identifying an agent that binds a polypeptide, the polypeptide is a critical, preferably, an essential, polypeptide having structural similarity, for instance, at least about 95 percent structural similarity, with a polypeptide of the present invention.

The present invention is also directed to a method for decreasing the growth rate of a microbe. The method includes combining a microbe with an agent that binds to a polypeptide of the present invention. The microbe may by in vitro or in vivo.

The present invention is further directed to a method for making an H. influenzae with reduced virulence. The method includes altering a coding sequence in an H. influenzae to include a mutation, and determining if the H. influenzae including the mutation has reduced virulence compared to an H. influenzae that does not include the mutation. The non-mutagenized coding sequence can include a coding sequence present at a polynucleotide of the present invention. The mutation may be, for instance; a deletion mutation, an insertion mutation, a nonsense mutation, and a missense mutation. The present invention also includes a H. influenzae having reduced virulence, and a vaccine composition that includes the H. influenzae.

### BRIEF DESCRIPTION OF THE FIGURES

FIG. 1. Overview of strategy for vector-based targeted coding sequence disruption in H. influenzae.

FIG. 2. Sequence of the galK coding sequence and flanking regions. The galK coding sequence is in lowercase letters and the PCR primers are underlined.

FIG. 3. Sequence of the tmk coding sequence and flanking regions. The tmk coding sequence is in lowercase letters and the PCR primers are underlined.

FIG. 4. Overview of strategy for the direct mutagenesis of a target PCR product. The triangle represents a transposon.

FIG. 5. Sequence of the aroC coding sequence and flanking regions. The aroC coding sequence is in lowercase letters and the primer sequences are underlined.

FIG. 6. Sequence of the coaD coding sequence and flanking regions. The coaD coding sequence is in lowercase letters and the primer sequences are underlined.

FIG. 7. Sequence of the rhlB coding sequence and flanking regions. The rhlB coding sequence is in lowercase letters and the primer sequences are underlined.

FIG. 8. Sequence of the ribB coding sequence and flanking regions. The ribB coding sequence is in lowercase letters and the primer sequences are underlined.

FIG. 9. Sequence of the ribF coding sequence and flanking regions. The ribF coding sequence is in lowercase letters and the primer sequences are underlined.

FIG. 10. Sequence of the yihZ coding sequence and flanking regions. The yihZ coding sequence is in lowercase letters and the primer sequences are underlined.

FIG. 11. Sequence of the yfgB coding sequence and flanking regions. The yfgB coding sequence is in lowercase letters and the primer sequences are underlined.

FIG. 12. Sequence of the yrdC coding sequence and flanking regions. The yrdC coding sequence is in lowercase letters and the primer sequences are underlined.

FIG. 13. Sequence of the suhB coding sequence and flanking regions. The suhB coding sequence is in lowercase letters and the primer sequences are underlined.

FIG. 14 Sequence of the yhbJ coding sequence and flanking regions. The yhbJ coding sequence is in lowercase letters and the primer sequences are underlined.

FIG. 15 Sequence of the yihZ, yfgB and yhbJ genes showing the location of the transposon (∇) inserted within each gene.

FIG. 16. Sequence of the efp coding sequence and flanking regions. The efp coding sequence is in lowercase letters and the PCR primers are underlined.

FIG. 17. Sequence of the fba coding sequence and flanking regions. The fba coding sequence is in lowercase letters and the PCR primers are underlined.

FIG. 18. Sequence of the fmt coding sequence and flanking regions. The fmt coding sequence is in lowercase letters and the PCR primers are underlined.

FIG. 19. Sequence of the IF-1 coding sequence and flanking regions. The IF-1 coding sequence is in lowercase letters and the PCR primers are underlined.

FIG. 20. Sequence of the IF-2 coding sequence and flanking regions. The IF-2 coding sequence is in lowercase letters and the PCR primers are underlined.

FIG. 21. Sequence of the IF-3 coding sequence and flanking regions. The IF-3 coding sequence is in lowercase letters and the PCR primers are underlined.

FIG. 22. Sequence of the ispA coding sequence and flanking regions. The ispA coding sequence is in lowercase letters and the PCR primers are underlined.

FIG. 23. Sequence of the ispB coding sequence and flanking regions. The ispB coding sequence is in lowercase letters and the PCR primers are underlined.

FIG. 24. Sequence of the nusA coding sequence and flanking regions. The nusA coding sequence is in lowercase letters and the PCR primers are underlined.

FIG. 25. Sequence of the pth coding sequence and flanking regions. The pth coding sequence is in lowercase letters and the PCR primers are underlined.

FIG. 26. Sequence of the tmk coding sequence and flanking regions. The tmk coding sequence is in lowercase letters and the PCR primers are underlined.

FIG. 27 Sequence of the trxB coding sequence and flanking regions. The trxB coding sequence is in lowercase letters and the PCR primers are underlined.

FIG. 28. Sequence of the uppS coding sequence and flanking regions. The uppS coding sequence is in lowercase letters and the PCR primers are underlined.

FIG. 29. Sequence of the L27 coding sequence and flanking regions. The L27 coding sequence is in lowercase letters and the PCR primers are underlined.

FIG. 30. Sequence of the lepA coding sequence and flanking regions. The lepA coding sequence is in lowercase letters and the PCR primers are underlined.

FIG. 31. Sequence of the ispA, L27 and trxB coding sequences showing the location of the transposon (∇) inserted within each coding sequence.

FIG. 32. Sequence of the efp and pth genes showing the location of the transposons (∇) inserted within each gene. Efp was determined to be non-essential, whereas IF-1 and pth were determined to be essential.

FIG. 33. Sequence of the alr coding sequence and flanking regions. The alr coding sequence is in lowercase letters and the PCR primers are underlined.

FIG. 34. Sequence of the amiB coding sequence and flanking regions. The amiB coding sequence is in lowercase letters and the PCR primers are underlined.

FIG. 35. Sequence of the dacA coding sequence and flanking regions. The dacA coding sequence is in lowercase letters and the PCR primers are underlined.

FIG. 36. Sequence of the dacB coding sequence and flanking regions. The dacB coding sequence is in lowercase letters and the PCR primers are underlined.

FIG. 37. Sequence of the ddlB coding sequence and flanking regions. The ddlB coding sequence is in lowercase letters and the PCR primers are underlined.

FIG. 38. Sequence of the ftsI coding sequence and flanking regions. The ftsI coding sequence is in lowercase letters and the PCR primers are underlined.

FIG. 39. Sequence of the glmS coding sequence and flanking regions. The glmS coding sequence is in lowercase letters and the PCR primers are underlined.

FIG. 40. Sequence of the glmU coding sequence and flanking regions. The glmU coding sequence is in lowercase letters and the PCR primers are underlined;

FIG. 41. Sequence of the glnA coding sequence and flanking regions. The glnA coding sequence is in lowercase letters and the PCR primers are underlined.

FIG. 42. Sequence of the lpp coding sequence and flanking regions. The lpp coding sequence is in lowercase letters and the PCR primers are underlined.

FIG. 43. Sequence of the mepA coding sequence and flanking regions. The mepA coding sequence is in lowercase letters and the PCR primers are underlined.

FIG. 44. Sequence of the mtgA coding sequence and flanking regions. The mtgA coding sequence is in lowercase letters and the PCR primers are underlined.

FIG. 45. Sequence of the mraY coding sequence and flanking regions. The mraY coding sequence is in lowercase letters and the PCR primers are underlined.

FIG. 46. Sequence of the murB coding sequence and flanking regions. The murB coding sequence is in lowercase letters and the PCR primers are underlined.

FIG. 47. Sequence of the murC coding sequence and flanking regions. The murC coding sequence is in lowercase letters and the PCR primers are underlined.

FIG. 48. Sequence of the murD coding sequence and flanking regions. The murD coding sequence is in lowercase letters and the PCR primers are underlined.

FIG. 49. Sequence of the murE, murF and mraY coding sequences and flanking regions. The murE coding sequence (nucleotides 936-2429) is in lowercase letters, the murF coding sequence (nucleotides 2443-3809) is in italisized lowercase letters, the mraY coding sequence is in italisized uppercase letters, and the PCR primers are underlined.

FIG. 50. Sequence of the murG coding and flanking regions. The murG coding sequence is in lowercase letters and the PCR primers are underlined.

FIG. 51. Sequence of the murI coding sequence and flanking regions. The murI coding sequence is in lowercase letters and the PCR primers are underlined.

FIG. 52. Sequence of the murZ coding sequence and flanking regions. The murZ coding sequence is in lowercase letters and the PCR primers are underlined.

FIG. 53. Sequence of the nagA coding sequence and flanking regions. The nagA coding sequence is in lowercase letters and the PCR primers are underlined.

FIG. 54. Sequence of the pal coding sequence and flanking regions. The pal coding sequence is in lowercase letters and the PCR primers are underlined.

FIG. 55. Sequence of the pbpG coding sequence and flanking regions. The pbpG coding sequence is in lowercase letters and the PCR primers are underlined.

FIG. 56. Sequence of the pbp2 coding sequence and flanking regions. The pbp2 coding sequence is in lowercase letters and the PCR primers are underlined.

FIG. 57. Sequence of the ponA coding sequence and flanking regions. The ponA coding sequence is in lowercase letters and the PCR primers are underlined.

FIG. 58. Sequence of the ponB coding sequence and flanking regions. The ponB coding sequence is in lowercase letters and the PCR primers are underlined.

FIG. 59. Sequence of the slt coding sequence and flanking regions. The slt coding sequence is in lowercase letters and the PCR primers are underlined.

FIG. 60. Biosynthetic pathway for H. influenzae peptidoglycan biosynthesis. The genes encoding essential enzymes (alr, ddlB, ftsI, glmU, mraY, murB, murC, murD, murE, murF, murG, murI, murZ, and nagA), and the genes encoding non-essential enzymes (glms, ponA, ponB, pbp2, dacA, dacB, pbpG and amiB) are depicted at the appropriate step in the pathway. 6-P, 6-phosphate; 1-P, 1-phosphate; GlcNAc, N-acetylglucosamine; MurNAc, N-acetylmuramic acid; UDP, uridine diphosphate; L-ala, L-alanine; D-ala, D-alanine; L-glu, L-glutamate; D-Glu, D-glutamate. The ovals attached to MurNAc signify the amino acids added to MurNAc.

FIG. 61. The H. influenzae cell wall biosynthetic operon. The bars above the operon show the PCR fragment generated by the N/C primer pair for each of the eight genes tested. The triangles represent sequence mapped Tn insertions. FtsL, ftsW and ftsQ are cell division proteins. Chp represents a conserved hypothetical protein.

FIG. 62. Diagram showing the N/C primer pair used to synthesize the murE/murF PCR fragment and the K/Z primer pairs used to screen the resulting recombinants. Triangles, transposons mapped to the 13 bases between murE and murF, and to the last 32 bases of the murF coding sequence.

FIG. 63. Predicted amino acid sequences of coding sequences. The name of the coding sequence and the locus is above each amino acid sequence.

FIG. 64. Oligonucleotides used for the construction of the mrsA deletion fragment. Sequences derived from the chloramphenical (CM) coding sequence are underlined. Sequences derived from the mrsA gene are italicized in bold. Oligonucleotides used to screen for and to confirm the deletion of the mrsA gene.

FIG. 65. Outline of the protocol for the construction of the mrsA deletion fragment.

FIG. 66. Sequence of the mrsA coding sequence and flanking regions. The mrsA coding sequence is in lowercase letters. The PCR primers A1 and C2, used to generate the deletion fragments, are boxed. The mrsA sequences used as part of the fusion primers A2 and C1 are underlined.

FIG. 67. Sequence of the CM coding sequence and flanking regions from pACYC184. The CM coding sequence is in lowercase letters. CM sequences used in the fusion primers B1 and B2 are underlined. Sequences used as primers to confirm the substitution of the CM coding sequence (CM-NR and CM-CR) are in bold italics.

FIG. 68. Sequence of the mrsA coding sequence showing the PCR primers used for the insertional inactivation of the remaining mrsA coding sequence. The Z primer was also used to confirm the deletion of the mrsA coding sequence.

FIG. 69. Sequence of mrsA showing the location of the Tn inserts in the H. influenzae wild type strain. Insertions 1 and 3 are located in HI1337, and insertions 15 and 19 are located in HI1463.

FIG. 70. Sequence of the rpL19 (HI0201, rplS) and rpS16 (HI0204, rpsP) genes and flanking regions. The rpL19 coding sequence is in lowercase letters (nucleotides 2636-2986), the rpS16 coding sequence is in lowercase italicized letters (nucleotides 1001-1249), and the PCR primers are underlined.

FIG. 71. Sequence of the rpL1 (HI0516, rplA) and rpL11 (HI0517, rplK) genes and flanking regions. The rpL1 coding sequence is in lowercase letters (nucleotides 838-1266), the rpL11 coding sequence is in lowercase italicized letters (nucleotides 1271-1960), and the PCR primers are underlined.

FIG. 72. Sequence of the rpS21 (HI0531, rpsU) genes and flanking regions. The rpS21 coding sequence is in lowercase letters (nucleotides 1002-1217) and the PCR primers are underlined.

FIG. 73. Sequence of the rpS6 (HI0547, rpsF) and rpS18 (HI0545, rpsR) genes and flanking regions. The rpS6 coding sequence is in lowercase letters (nucleotides 1084-1461), the rpS18 coding sequence is in lowercase italicized letters (nucleotides 1787-2014), and the PCR primers are underlined.

FIG. 74. Sequence of the rpS7 (HI0580, rpsG) and rpS12 (HI0581, rpsL) genes. and flanking regions. The rpS7 coding sequence is in lowercase letters (nucleotides 1530-2000), the rpS12 coding sequence is in lowercase italicized letters (nucleotides 999-1373), and the PCR primers are underlined.

FIG. 75. Sequence of the rpL10 (HI0640, rplJ) and rplL7/12 (HI0641, rplL) genes and flanking regions. The rpL10 coding sequence is in lowercase letters (nucleotides 1002-1493), the rpL7/12 gene sequence is in lowercase italicized letters (nucleotides 1548-1919), and the PCR primers are underlined.

FIG. 76. Sequence of the rpS10 (HI0776, rpsJ), rpL3 (HI0777, rplC), rpL4 (HI0778, rplD), rpL23 (HI0779, rplW), rpL2 (HI0780, rplB), rpS19 (HI0781, rpsS), rpL22 (HI0782, rplV), rpS3 (HI0783, rpsC), rpL16 (HI0784, rplP), rpL29 (HI0785, rpmC), and rpS17 (HI0786, rpsQ) genes and flanking regions. All of the coding sequences are shown sequentially in lowercase letters: rpS10 (nucleotides 731-1087), rpL3 (nucleotides 1104-1730), rpL4 (nucleotides 1746-2348), rpL23 (nucleotides 2345-2644), rpL2 (nucleotides 2622-3483), rpS19 (nucleotides 3509-3784), rpL22 (nucleotides 3796-4128), rpS3-(nucleotides 4146-4853), rpL16 (nucleotides 4867-5277), rpL29 (nucleotides 5277-5468), and rpS17 (nucleotides 5468-5725). The PCR primers are underlined.

FIG. 77. Sequence of the rpL14 (HI0788, rplN), rpl24 (HI0789, rplX), rpL5 (HI0790, rplE), rpS14 (HI0791, rpsN), rpS8 (HI0792, rpsH), rpL6 (HI0793, rplF), rpL18 (HI0794, rplR), rpS5 (HI0795, rpsE), rpL30 (HI0796, rpmD), and rpL15 (HI0797, rplO) genes and flanking regions. All of the coding sequences are shown sequentially in lowercase letters: rpL14 (nucleotides 1002-1373), rpL24 (nucleotides 1384-1695), rpL5 (nucleotides 1713-2252), rpS14 (nucleotides 2264-2589), rpS8 (nucleotides 2602-2988), rpL6 (nucleotides 3014-3547), rpL18 (nucleotides 3561-3914), rpS5 (nucleotides 3929-4429), rpL30 (nucleotides 4436-4615), and rpL15 (nucleotides 4619-5053). The PCR primers are underlined.

FIG. 78. Sequence of the rpL36 (HI0798.1, rpmJ), rpS13 (HI0799, rpsM), rpS11 (HI0800, rpsK), rpS4 (HI0801, rpsD) and rpL17 (HI0803, rplQ) genes and flanking regions. All of the coding sequences are shown sequentially in lowercase letters: rpL36 (nucleotides 1362-1475), rpS13 (nucleotides 1615-1983), rpS11 (nucleotides 1986-2375), rpS4 (nucleotides 2403-3023), and rpL17 (nucleotides 4082-4468). The PCR primers are underlined.

FIG. 79. Sequence of the rpL21 (HI0880, rplU) gene and flanking regions. The rpL21 coding sequence is in lowercase letters (nucleotides 669-980) and the PCR primers are underlined.

FIG. 80. Sequence of the rpS2 (HI0913, rpsB) gene and flanking regions. The rpS2 coding sequence is in lowercase letters (nucleotides 1002-1757) and the PCR primers are underlined.

FIG. 81. Sequence of the rpL28 (HI0951, rpmB) gene and flanking regions. The rpL28 coding sequence is in lowercase letters (nucleotides 961-1197), and the PCR primers are underlined.

FIG. 82. Sequence of the rpS20 (HI0965, rpsT) gene and flanking regions. The rpS20 gene sequence is in lowercase letters (nucleotides 1002-1271) and the PCR primers are underlined.

FIG. 83. Sequence of the rpL43 (HI0998, rpmH) gene and flanking regions; The rpL34 coding sequence is in lowercase letters (nucleotides 1001-1135) and the PCR primers are underlined.

FIG. 84. Sequence of the rpS1 gene (HI1220, rpsA) and flanking regions. The rpS1 coding sequence is in lowercase letters (nucleotides 1002-2651) and the PCR primers are underlined.

FIG. 85. Sequence of the rpL35 (HI1319, rpmI) and rpL20 (HI1320, rplT) genes and flanking regions. The rpL35 coding sequence is in lowercase letters (nucleotides 948-1217), the rpL20 coding sequence is in lowercase italicized letters (nucleotides 1284-1637), and the PCR primers are underlined.

FIG. 86. Sequence of the rpS15 (HI1328, rpsO) gene and flanking regions. The rpS15 coding sequence is in lowercase letters (nucleotides 1002-1271) and the PCR primers are underlined.

FIG. 87. Sequence of the rpS9 (HI1442, rpsI) and rpL13 (HI1443, rpsM) genes and flanking regions. The rpS9 coding sequence is in lowercase letters (nucleotides 1608-2000), the rpL13 gene sequence is in lowercase italicized letters (nucleotides 1163-1591), and the PCR primers are underlined.

FIG. 88. Sequence of the rpL25 gene (HI1630, rplY) and flanking regions. The rpL25 coding sequence is in lowercase letters (nucleotides 1001-1288) and the PCR primers are underlined.

FIG. 89. Sequence of the dnaE gene and flanking regions. The dnaE coding sequence is in lowercase letters (nucleotides 1001-4480) and the PCR primers are underlined.

FIG. 90. Sequence of the mesJ gene and flanking regions. The mesJ coding sequence is in lowercase letters (nucleotides 1001-2293) and the PCR primers are underlined.

FIG. 91. Sequence of the cdsA gene (110919) and flanking regions. The cdsA coding sequence is in lowercase letters (nucleotides 1101-1967) and the PCR primers are underlined.

FIG. 92. Sequence of the pyrH gene (HI1065) and flanking regions. The pyrH coding sequence is in lowercase letters (nucleotides 1604-2317) and the PCR primers are underlined.

FIG. 93. Sequence of the coaE gene (HI10890) and flanking regions. The coaE coding sequence is in lowercase letters (nucleotides 981-1613) and the PCR primers are underlined.

FIG. 94. Sequence of the emrB gene (HI0897) and flanking regions. The emrB coding sequence is in lowercase letters (nucleotides 1001-2533) and the PCR primers are underlined.

FIG. 95. Predicted amino acid sequences of coding sequences. The name of the coding sequence and the locus is above each amino acid sequence.

FIG. 96. Nucleotide sequences of open reading frames. The name of the open reading frame and the locus is above each nucleotide sequence.

FIG. 97. A. DNA sequence (SEQ ID NO:500) of tmk encoding a C-terminal hexahistidine tag. The 5′ and 3′ primers are underlined in italics. Sequence in the coding strand that is not native tmk but was added in the process of cloning is further indicated in bold. Restriction sites used for cloning are indicated. B. Amino acid sequence (SEQ ID NO:501) encoded by the coding region of SEQ ID NO:500. Non-native amino acids added as a result of cloning are indicated in bold. C. Plasmid Map of H. influenzae Tmk 6XCT in the pET15b Expression Vector.

### DETAILED DESCRIPTION OF PREFERRED EMBODIMENTS OF THE INVENTION

The sequence of the H. influenzae genome has been determined and includes about 1,738 coding sequences (see, for instance, Fleischmann et al., Science, 269, 496-512 (1995); GenBank Accession Number L42023 and the Accession Numbers cited therein; and at The Institute for Genomic Research (TIGR) comprehensive microbial resource, Haemophilus influenzae KW20 genome page (www.tigr.org/tigr-scripts/CMR2/GenomePage3.spl?database=ghi)). As used herein, the terms “coding sequence,” “coding region,” and “open reading frame” are used interchangeably herein and refer to a nucleotide sequence that encodes a polypeptide and, when placed under the control of appropriate regulatory sequences, expresses the encoded polypeptide. The boundaries of a coding region are generally determined by a translation start codon at its 5′ end and a translation stop codon at its 3′ end. A regulatory sequence is a nucleotide sequence that regulates expression of a coding region to which it is operably linked. Nonlimiting examples of regulatory sequences include promoters, transcription initiation sites, translation start sites, translation stop sites, and terminators. “Operably linked” refers to a juxtaposition wherein the components so described are in a relationship permitting them to function in their intended manner. A regulatory sequence is “operably linked” to a coding region when it is joined in such a way that expression of the coding region is achieved under conditions compatible with the regulatory sequence.

The function of some coding sequences of the H. influenzae genome has been hypothesized by comparing an H. influenzae coding sequence with a second coding sequence from another organism, where the second coding sequence has a known function. The putative function of many of the H. influenzae coding sequences is described in Fleischmann et al. (Science, 269, 496-512 (1995)), GenBank Accession Number L42023, and at The Institute for Genomic Research (TIGR) comprehensive microbial resource, Haemophilus influenzae KW20 genome page (www.tigr.org/tigr-scripts/CMR2/GenomePage3.spl?database=ghi). This subset of coding sequences is referred to herein as “known coding sequences.” However, even though the function of these coding sequences can be hypothesized, for many it is unknown if they are required for bacterial growth. Those known coding sequences that are required for bacterial growth are potential novel targets for antimicrobial therapy.

At this time, it is not possible to predict the function of some of the polypeptides that the approximately 1,738 coding sequences of the H. influenzae genome are predicted to encode. This subset of coding sequences is referred to herein as “unknown coding sequences.” Among the unknown coding sequences in the H. influenzae genome, those that are required for cell growth are potential novel targets for antimicrobial therapy.

As used herein, a “critical coding sequence” encodes a polypeptide that is required for a bacterial cell, preferably H. influenzae, H. ducreyi, or H. aegyptius, more preferably, H. influenzae, to grow at a normal growth rate in vitro or in vivo, preferably in vitro. A coding sequence is a critical coding sequence when mutagenesis of the coding sequence in a bacterial cell decreases the growth rate of the bacterial cell to, in increasing levels of preference, less than about 50%, less than about 60%, less than about 80%, most preferably, less than about 90% of the growth rate of the bacterial cell that does not contain the mutated coding sequence. Methods of measuring the growth rate of microbes are well known and routine in the art and include, for instance, measurement by changes in optical density of a liquid culture as a function of time, or measurement by changes in colony diameter as a function of time. A critical coding sequence may encode a polypeptide having a known function, or in some aspects of the invention, encode a polypeptide having an unknown function. Preferably, a critical coding sequence encodes a polypeptide having a known function.

A polypeptide encoded by a critical coding sequence is referred to herein as a “critical polypeptide.” As used herein, the term “polypeptide” refers to a polymer of amino acids and does not refer to a specific length of a polymer of amino acids. Thus, for example, the terms peptide, oligopeptide, protein, and enzyme are included within the definition of polypeptide. This term also includes post-expression modifications of the polypeptide, for example, glycosylations, acetylations, phosphorylations and the like.

As used herein, growth of a microbe “in vitro” refers to growth in, for instance, a test tube or on an agar plate. Growth of a microbe “in vivo” refers to growth in, for instance, a cultured cell or in an animal. As used herein, the term “microbe” and “bacteria” are used interchangeably and include single celled prokaryotic and lower eukaryotic (e.g., fungi) organisms, preferably prokaryotic organisms.

Preferably, a critical coding sequence is an essential coding sequence. An “essential coding sequence,” as used herein, is a coding sequence that encodes a polypeptide that is essential for the bacterial cell, preferably H. influenzae, H. ducreyi, or H. aegyptius, more preferably, H. influenzae, to grow in vitro or in vivo, preferably in vitro. Such polypeptides are referred to herein as “essential polypeptides.” An essential coding sequence may encode a polypeptide having an unknown function, or in some aspects of the invention, encode a polypeptide having a known function. Preferably, an essential coding sequence encodes a polypeptide having a known function.

Identification of these critical coding sequences, preferably essential coding sequences, provides a means for discovering new agents with different targets and mechanisms of action compared to existing agents that are used to inhibit bacteria, preferably H. influenzae, H. ducreyi, or H. aegyptius, more preferably, H. influenzae. As used herein, the term “agent” refers to chemical compounds, including, for instance, an organic compound, an inorganic compound, a metal, a polypeptide, a non-ribosomal polypeptide, a polyketide, or a peptidomimetic compound that binds to a particular polypeptide or nucleotide sequence. The terms “binds to a polypeptide” and “binds a polypeptide” refer to a condition of proximity between an agent and a polypeptide. The association may be non-covalent, wherein the juxtaposition is energetically favored by hydrogen bonding, van der Waals forces, or electrostatic interactions, or it may be covalent. The identification of coding sequences of microbes, preferably H. influenzae, H. ducreyi, or H. aegyptius, more preferably, H. influenzae, that are useful in the present invention can begin by identifying coding sequences predicted to encode a polypeptide. The coding sequences can be identified in databases, including, for instance, the GenBank database, the TIGR Comprehensive Microbial Resource, and the Kyoto Encyclopedia of Genes and Genores. The identification of such coding sequences can include constructing contigs from data present in such databases.

The data obtained from the databases may contain the nucleotide sequence of genomic clones and predicted open reading frames. However, even though the putative coding sequences may have been known, there was no indication that the coding sequences were in fact expressed, or in fact critical coding sequences. For instance, there is limited data known in the art regarding regulatory regions required for the transcription of a nucleotide sequence in H. influenzae. Moreover, prior to the experiments described herein, there was generally no evidence that the critical coding sequences and essential coding sequences identified herein were actually expressed. Thus, a person of ordinary skill, having the polynucleotide sequence of a genomic clone, would not be able to predict that an open reading frame would be transcribed, or that a coding sequence was critical, preferably, essential.

Typically, whether a coding sequence is a critical coding sequence, preferably, an essential coding sequence, can be determined by inactivating the coding sequence in a bacterial cell and determining the growth rate of the bacterial cell. Growth can be measured in vitro or in vivo, preferably in vitro. Inactivating a coding sequence may be done by mutating a coding sequence present in a bacterial cell. Mutations include, for instance, a deletion mutation (i.e., the deletion of nucleotides from the coding sequence), an insertion mutation (i.e., the insertion of additional nucleotides, for instance, a transposon, into the coding sequence), a nonsense mutation (i.e., changing a nucleotide of a codon so the codon encodes a different amino acid), and a missense mutation (i.e., changing a nucleotide of a codon so the codon functions as a stop codon). Some insertion mutations and some deletion mutations result in frame-shift mutations. Preferably, a coding sequence in a bacterial cell is engineered to contain an insertion.

Methods for engineering a coding sequence to contain an insertion are known in the art. Preferably, the insertion is a transposon. In general, a selected coding sequence can be subjected to transposon mutagenesis by isolating or synthesizing the coding sequence by methods known in the art, including, for instance, the polymerase chain reaction (PCR). Preferably, the coding sequence includes about 1,000 base pairs (bp) flanking the coding sequence (i.e., about 500 bp upstream and about 500 bp downstream of the coding sequence), more preferably, about 2,000 bp flanking the coding sequence. Optionally, the coding sequence may be ligated to a vector. Preferably, a vector is a suicide vector, i.e., it is unable to replicate in H. influenzae. An example of a suicide plasmid that can be used with H. influenzae is pBR322. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual., Cold Spring Harbor Laboratory Press (1989). A vector can provide for further cloning (amplification of the polynucleotide), i.e., a cloning vector, or for expression of the polypeptide encoded by the coding region, i.e., an expression vector. Preferably the vector is a plasmid.

The coding sequence is subjected to in vitro transposon mutagenesis using routine methods known in the art. Preferably, in vitro mutagenesis is accomplished by using Tn7, available under the trade designation GPS-M (New England Biolabs, Beverly, Mass.), or using Tn5 as described by Goryshin and Reznikoff, J. Biol. Chem., 273, 7367-7374 (1998), and available under the trade designation EZ::TN (Epicentre, Madison, Wis.). The transposon typically includes a coding sequence that encodes a selectable marker. A selectable marker can render a cell resistant to an antibiotic, for example kanamycin, ampicillin, chloramphenicol, tetracycline, and neomycin.

Following mutagenesis, the coding sequence may be introduced to a bacterial cells, preferably an H. influenzae strain, using methods known in the art, and the transformed strains are incubated under conditions that select for those transformants containing the transposon present in the chromosome. A solid medium (e.g., media containing about 1.5 % agar) or liquid medium may be used. Examples of rich media include brain heart infusion, and others are known in the art (see, for instance, Atlas, Handbook of Microbiological Media, 2 ed., CRC Press (1997)). Preferably, the medium used is solid, containing brain heart infusion supplemented with about 5% Fildes Enrichment. Typically, at least about 40 individual transformants are subjected to DNA amplification of a region of the selected coding sequence that corresponds to about the first 300 bp of the coding sequence, more preferably, the first 500 bp of the coding sequence. If no insertions are found in this region, and subsequent analysis indicates transposon insertions are present upstream or downstream of the coding sequence, the coding sequence that was the target of mutagenesis is considered essential. If insertions are found in this region, but the transformants containing insertions in this region have a decreased growth rate, the coding sequence that was the target of mutagenesis is considered to be a critical coding sequence.

Using these methods, the following critical coding sequences have been identified: the coding sequence present in SEQ ID NO: 11, 16, 26, 31, 46, 64, 69, 74, 79, 84, 89, 99, 104, 109, 114, 124, 129, 145, 150, 165, 170, 180, 185, 200, 205, 210, 215, 220, 232, 237, 242, 247, 252, 267, 357, 389-443, nucleotides 936-2429 of SEQ ID NO:225, and nucleotides 2443-3809 of SEQ ID NO:225. The polypeptides encoded by these coding sequences are SEQ ID NO: 282, 283, 285, 286, 289, 292, 293, 294, 295, 296, 297, 299, 300, 301, 302, 304, 305, 307, 308, 311, 312, 314, 315, 318, 321, 322, 323, 324, 327, 328, 329, 340, 341, 343, 345, 444-498, 325, and 326, respectively. Using these methods, the following essential coding sequences have been identified: the coding sequence present in SEQ ID NO: 11, 16, 26, 31, 46, 69, 74, 79, 84, 89, 104, 109, 114, 124, 145, 165, 170, 180, 205, 210, 215, 220, 232, 237, 242, 247, 252, 357, 389-443, nucleotides 936-2429 of SEQ ID NO:225, and nucleotides 2443-3809 of SEQ ID NO:225. The polypeptides encoded by these coding sequences are SEQ ID NO: 282, 283, 285, 286, 289, 293, 294, 295, 296, 297, 300, 301, 302, 304, 307, 311, 312, 314, 321, 322, 323, 324, 327, 328, 329, 340, 341, 345, 444-498, 325, and 326, respectively.

The coding sequences of the present invention include critical coding sequences, preferably, essential coding sequences, that are similar to the coding sequences present in SEQ ID NO: 11, 16, 26, 31, 46, 64, 69, 74, 79, 84, 89, 99, 104, 109, 114, 124, 129, 145, 165, 170, 180, 205, 210, 215, 220, 232, 237, 242, 247, 252, 357, 150, 185, 200, 267, 389-443, nucleotides 936-2429 of SEQ ID NO:225, nucleotides 2443-3809 of SEQ ID NO:225, or the complement thereof. The similarity is referred to as structural similarity and is determined by aligning the residues of the two polynucleotides (i.e., the nucleotide sequence of the candidate coding sequence and the nucleotide sequence of the coding region of SEQ ID NO:11, 16, 26, 31, 46, 64, 69, 74, 79, 84, 89, 99, 104, 109, 114, 124, 129, 145, 165, 170, 180, 205, 210, 215, 220, 232, 237, 242, 247, 252, 357, 150, 185, 200, 267, 389-443, nucleotides 936-2429 of SEQ ID NO:225, nucleotides 2443-3809 of SEQ ID NO:225, or the complement thereof) to optimize the number of identical nucleotides along the lengths of their sequences; gaps in either or both sequences are permitted in making the alignment in order to optimize the number of shared nucleotides, although the nucleotides in each sequence must nonetheless remain in their proper order. A candidate coding region is the coding region being compared to a coding region present in SEQ ID NO:11, 16, 26, 31, 46, 64, 69, 74, 79, 84, 89, 99, 104, 109, 114, 124, 129, 145, 165, 170, 180, 205, 210, 215, 220, 232, 237, 242, 247, 252, 357, 150, 185, 200, 267, 389-443, nucleotides 936-2429 of SEQ ID NO:225, nucleotides 2443-3809 of SEQ ID NO:225, or the complement thereof. A candidate coding region can be isolated from a microbe, preferably H. influenzae, or can be produced using recombinant techniques, or chemically or enzymatically synthesized. Preferably, two coding regions are compared using the blastn program of the BLAST search algorithm, which is described by Altshul et al., (Nucl. Acids Res., 25, 3389-3402 (1997)), and available at the National Center for Biotechnology Information (for instance, www.ncbi.nlm.nih.gov/BLAST/, or www.ncbi.nlm.nih.gov/Microb_blast/unfinishedgenome.html). Preferably, the default values for all BLAST search parameters are used. In the comparison of two coding regions using the BLAST search algorithm, structural similarity is referred to as “identities.” Preferably, a polynucleotide includes a coding region having a structural similarity with the coding region of SEQ ID NO:11, 16, 26, 31, 46, 64, 69, 74, 79, 84, 89, 99, 104, 109, 114, 124, 129, 145, 165, 170, 180, 205, 210, 215, 220, 232, 237, 242, 247, 252, 357, 150, 185, 200, 267, 389-443, nucleotides 936-2429 of SEQ ID NO:225, nucleotides 2443-3809 of SEQ ID NO:225, or the complement thereof, of, in increasing order of preference, at least about 40%, at least about 60%, at least about 80%, at least about 90%, most preferably at least about 95% identity.

Typically, such a candidate coding region having structural similarity to a coding region of one of the listed sequences has activity, i.e., it is a critical coding region or an essential coding region. Whether such a candidate coding region is critical or essential can be determined by evaluating whether the candidate coding region encodes a polypeptide that is able to complement a mutation of the appropriate coding region in H. influenzae, preferably the H. influenzae available from the American Type Culture Collection as ATCC 51907. For instance, to determine if a coding region having structural similarity to the coding region present in SEQ ID NO:64 is a critical coding region, the coding region can be expressed in an H. influenzae containing a mutation in the coding region present at SEQ ID NO:64. If the growth rate of the H. influenzae is restored, then the candidate coding region is a critical coding region. Likewise, to determine if a coding region having structural similarity to the coding region present in SEQ ID NO:11 is an essential coding region, the coding region can be expressed in a H. influenzae containing a mutation in the coding region present at SEQ ID NO:11. If the growth rate of the H. influenzae is restored, then the candidate coding region is an essential coding region. For example, a candidate coding region can be introduced into H. influenzae on a plasmid and expressed, and using the methods described herein, the chromosomal copy of the appropriate coding region can be inactivated. If insertions of the chromosomal copy of the appropriate coding region are identified, then the candidate coding region is an essential coding region.

Preferably the polynucleotides of the present invention are isolated. As used herein, an “isolated” polypeptide or polynucleotide means a polypeptide or polynucleotide that has been either removed from its natural environment, produced using recombinant techniques, or chemically or enzymatically synthesized. Typically, an isolated polynucleotide of the present invention does not include the entire genome of the microbe, preferably, H. influenzae, from which the polynucleotide was obtained. Preferably, a polypeptide or polynucleotide of this invention is purified, i.e., essentially free from any other polypeptides or polynucleotides and associated cellular products or other impurities.

The present invention includes the critical polypeptides and essential polypeptides encoded by the coding sequences of the present invention. Preferably, a polypeptide of the present invention is isolated, more preferably, purified. The critical, preferably, essential, polypeptides of the present invention include polypeptides that are similar to the polypeptides present in SEQ ID NO: 282, 283, 285, 286, 289, 292, 293, 294, 295, 296, 297, 299, 300, 301, 302, 304, 305, 307, 311, 312, 314, 321, 322, 323, 324, 327, 328, 329, 340, 341, 345, 325, 326, 292, 299, 305, 308, 315, 318, 343, 444-498. The similarity is referred to as structural similarity and is determined by aligning the residues of the two polypeptides (i.e., the amino acid sequence of the candidate polypeptide and the amino acid sequence of SEQ ID NO:282, 283, 285, 286, 289, 293, 294, 295, 296, 297, 300, 301, 302, 304, 307, 311, 312, 314, 321, 322, 323, 324, 327, 328, 329, 340, 341, 345, 325, 326, 292, 299, 305, 308, 315, 318, 343, 444-498 to optimize the number of identical amino acids along the lengths of their sequences; gaps in either or both sequences are permitted in making the alignment in order to optimize the number of shared amino acids, although the amino acids in each sequence must nonetheless remain in their proper order. A candidate amino acid sequence is the polypeptide being compared to one of SEQ ID NO:282, 283, 285, 286, 289, 293, 294, 295, 296, 297, 300, 301, 302, 304, 307, 311, 312, 314, 321, 322, 323, 324, 327, 328, 329, 340, 341, 345, 325, 326, 292, 299, 305, 308, 315, 318, 343, 444-498. A candidate amino acid sequence can be isolated from a microbe, preferably H. influenzae, or can be produced using recombinant techniques, or chemically or enzymatically synthesized. Preferably, two amino acid sequences are compared using the tblastn program of the BLAST search algorithm, which is described by Altshul et al., (Nucl. Acids Res., 25, 3389-3402 (1997)), and available at the National Center for Biotechnology Information (for instance, www.ncbi.nlm.nih.gov/Microb_blast/unfinishedgenome.html, or www.ncbi.nlm.nih.gov/BLAST/). Preferably, the default values for all BLAST search parameters are used. In the comparison of two amino acid sequences using the BLAST search algorithm, structural similarity is referred to as “identities.” Preferably, a polypeptide includes an amino acid sequence having a structural similarity with SEQ ID NO:282, 283, 285, 286, 289, 293, 294, 295, 296, 297, 300, 301, 302, 304, 307, 311, 312, 314, 321, 322, 323, 324, 327, 328, 329, 340, 341, 345, 325, 326, 292, 299, 305, 308, 315, 318, 343, 444-498 of, in increasing order of preference, at least about 56%, at least about 60%, at least about 70%, at least about 75%, at least about 80%, at least about 90%, most preferably at least about 95% identity.

Typically, such a candidate polypeptide having structural similarity to an amino acid sequence of the present invention has activity, i.e., it is a critical polypeptide or an essential polypeptide. Whether such a candidate polypeptide is critical or essential can be determined by evaluating whether it is able to complement a mutation of the appropriate coding region in H. influenzae, preferably the H. influenzae available from the American Type Culture Collection as ATCC 51907. For instance, to determine if a polypeptide having structural similarity to SEQ ID NO:292 is a critical polypeptide, the ability of the candidate polypeptide to complement an H. influenzae that contains a mutation such that it does not express a polypeptide having the sequence SEQ ID NO:292 may be determined. If the growth rate of the H. influenzae is restored, then the candidate polypeptide is a critical polypeptide. Likewise, to determine if a polypeptide having structural similarity to SEQ ID NO:282 is an essential polypeptide; the ability of the candidate polypeptide to complement an H. influenzae that contains a mutation such that it does not express a polypeptide having the sequence SEQ ID NO:282 may be determined. If the growth rate of the H. influenzae is restored, then the candidate coding region is an essential coding region. For example, a coding region encoding a candidate polypeptide can be introduced into H. influenzae on a plasmid and expressed, and using the methods described herein, the chromosomal copy of the appropriate coding region can be inactivated. If insertions of the chromosomal copy of the appropriate gene are identified, then the candidate polypeptide is an essential polypeptide.

Insertional inactivation of critical coding sequences, preferably, essential coding sequences, allows different classes of coding sequences to be identified. Examples of different classes include, for instance, coding sequences encoding proteins involved in cell surface metabolism, enzymes involved in cellular biosynthetic pathways including cell wall biosynthesis and assembly, components of the TCA cycle, proteins similar to oligopeptide transport proteins of the ATP-binding cassette (ABC) transporter superfamily, and involved in cellular regulatory and repair processes, and coding sequences affecting morphogenesis and cell division, secretion and sorting of proteins, and signal transduction systems.

The critical coding sequences, preferably, essential coding sequences may be cloned by PCR, using microbial, preferably H. influenzae, H. ducreyi, or H. aegyplius, more preferably, H. influenzae, genomic DNA as the template. When H. influenzae is used, genomic DNA may be obtained from the American Type Culture Collection as ATCC 51907D. For ease of inserting the open reading frame into vectors, preferably expression vectors, PCR primers may be chosen so that the PCR-amplified coding sequence has a restriction enzyme site at the 5′ end preceding the initiation codon ATG, and a restriction enzyme site at the 3′ end after the termination codon TAG, TGA or TAA. If desirable, the codons in the coding sequence may be changed, without changing the amino acids, to optimize expression of a polypeptide encoded by an essential coding sequence. For instance, if an essential coding sequence is to be expressed in E. coli, the codons of the coding sequence can be changed to comply with the E. coli codon preference (see, for instance, Grosjean and Fiers, Gene, 18, 199-209 (1982), and Konigsberg et al., Proc. Natl. Acad. Sci., USA, 80, 687-691 (1983)). Optimization of codon usage may lead to an increase in the expression of the encoded polypeptide when produced in a microbe other than the microbe from which the essential coding sequence was isolated. If the polypeptide is to be produced extracellularly, either in the periplasm of, for instance, E. coli or other bacteria, or into the cell culture medium, the coding sequence may be cloned without its initiation codon and placed into an expression vector behind a signal sequence.

Proteins may be produced in prokaryotic or eukaryotic expression systems using known promoters, vectors, and hosts. Such expression systems, promoters, vectors, and hosts are known to the art. A suitable host cell may be used for expression of the polypeptide, such as E. coli, other bactena, including Bacillus and H. influenzae, yeast, including Pichia pastoris and Saccharomyces cerevisiae, insect cells, or mammalian cells, including CHO cells, utilizing suitable vectors known in the art. Proteins may be produced directly or fused to a polypeptide, and either intracellularly or extracellularly by secretion into the periplasmic space of a bacterial cell or into the cell culture medium. Secretion of a protein typically requires a signal peptide (also known as pre-sequence); a number of signal sequences from prokaryotes and eukaryotes are known to function for the secretion of recombinant proteins. During the protein secretion process, the signal peptide is removed by signal peptidase to yield the mature protein.

The polypeptide encoded by a critical coding sequence, preferably, an essential coding sequence, may be isolated. To simplify the isolation process, a purification tag may be added either at the 5′ or 3′ end of the coding sequence. Commonly used purification tags include a stretch of six histidine residues (U.S. Pat. Nos. 5,284,933 and 5,310,663), a streptavidin-affinity tag described by Schmidt and Skerra, Protein Engineering, 6, 109-122 (1993), a FLAG peptide (Hopp et al, Biotechnology, 6, 1205-1210 (1988)), glutathione S-transferase (Smith and Johnson, Gene, 67, 31-40 (1988)), and thioredoxin (LaVallie et al., Bio/Technology, 11, 187-193 (1993)). To remove these tags, a proteolytic cleavage recognition site may be inserted at the fusion junction. Commonly used proteases are factor Xa, thrombin, and enterokinase.

The identification of critical coding sequences, preferably, essential coding sequences, renders them useful in methods of identifying new agents according to the present invention. Such methods include assaying potential agents for the ability to interfere with expression of a critical coding sequence, preferably, an essential coding sequence, thereby preventing the expression and decreasing the concentration of a polypeptide encoded by the coding sequence. Without intending to be limiting, it is anticipated that agents can~act by, for instance, interacting with a critical coding sequence, preferably, an essential coding sequence, interacting with a nucleotide sequence (e.g., a promoter sequence) that is adjacent to a critical coding sequence, preferably, an essential coding sequence, or inhibiting expression of a polypeptide involved in regulating expression of a critical coding sequence, preferably, an essential coding region. Agents that can be used to inhibit the expression of a critical coding sequence, preferably, an essential coding region include, for instance, the use of anti-sense polynucleotides that are complementary to the mRNA molecules transcribed from the coding sequence, and double stranded RNA (Fire et al., Nature, 391, 806-11 (1998)).

Such methods also include assaying potential agents for the ability to bind to a polypeptide encoded in whole or in part by a nucleotide sequence set forth in any one of the coding sequence present in SEQ ID NO:11, 16, 26, 31, 46, 64, 69, 74, 79, 84, 89, 99, 104, 109, 114, 124, 129, 145, 165, 170, 180, 205, 210, 215, 220, 232, 237, 242, 247, 252, 357, 150, 185, 200, 267, 389-443, nucleotides 936-2429 of SEQ ID NO:225, nucleotides 2443-3809 of SEQ ID NO:225, or the complementary strand thereof. Optionally, agents that bind to such a polypeptide can be further evaluated to determine if they inhibit the function of the polypeptide to which they bind.

A polypeptide produced by a critical coding sequence, preferably, an essential coding sequence, may be used in assays including, for instance, high throughput assays, to screen for agents that inhibit the function of the polypeptide. The sources for potential agents to be screened include, for instance, chemical compound libraries, fermentation media of Streptomycetes, other bacteria and fungi, and cell extracts of plants and other vegetations. For proteins with known enzymatic activity, assays may be established based on the activity, and a large number of potential agents can be screened for ability to inhibit the activity. Such assays are referred to herein as “enzyme assays.” Enzyme assays vary depending on the enzyme, and typically are known to the art.

For proteins that interact with another protein or nucleic acid, assays may be established to measure such interaction directly, and the potential agents screened for the ability to inhibit the binding interaction (referred to herein as “binding assays”). In another aspect of the invention, assays can be established allowing the identification of agents that bind to a polypeptide encoded by an essential coding sequence (referred to herein as “ligand binding assays”).

For proteins that interact with another protein or nucleic acid, such binding interactions may be evaluated indirectly using the yeast two-hybrid system described in Fields and Song, Nature, 340, 245-246 (1989), and Fields and Sternglanz, Trends in Genetics, 10, 286-292 (1994). The two-hybrid system is a genetic assay for detecting interactions between two polypeptides. It can be used to identify proteins that bind to a known protein of interest, or to delineate domains or residues critical for an interaction. Variations on this methodology have been developed to clone coding sequences that encode DNA-binding proteins, to identify polypeptides that bind to a protein, and to screen for drugs. The two-hybrid system exploits the ability of a pair of interacting proteins to bring a transcription activation domain into close proximity with a DNA-binding domain that binds to an upstream activation sequence (UAS) of a reporter coding sequence, and is generally performed in yeast. The assay requires the construction of two hybrid coding sequences encoding (1) a DNA-binding domain that is fused to a protein X, and (2) an activation domain fused to a protein Y. The DNA-binding domain targets the first hybrid protein to the UAS of the reporter coding sequence; however, because most proteins lack an activation domain, this DNA-binding hybrid protein does not activate transcription of the reporter coding sequence. The second hybrid protein, which contains the activation domain, cannot by itself activate expression of the reporter because it does not bind the UAS. However, when both hybrid proteins are present, the noncovalent interaction of protein X and protein Y tethers the activation domain to the UAS, activating transcription of the reporter coding sequence. When the polypeptide encoded by, for instance, an essential coding sequence (protein X, for example) is already known to interact with another protein or nucleic acid (protein Y, for example), this binding assay can be used to detect agents that interfere with the interaction of X and Y. Expression of the reporter coding sequence is monitored as different test agents are added to the system; the presence of an inhibitory agent inhibits binding and results in lack of a reporter signal.

When the function of a polypeptide encoded by, for instance, an essential coding sequence is unknown and no ligands are known to bind the polypeptide, the yeast two-hybrid assay can also be used to identify proteins that bind to the polypeptide. In an assay to identify proteins that bind to protein X (the target protein), a large number of hybrid coding sequences, each containing a different protein Y, are produced and screened in the assay. Typically, Y is encoded by a pool of plasmids in which total cDNA or genomic DNA is ligated to the activation domain. This system is applicable to a wide variety of proteins, and it is not even necessary to know the identity or function of protein Y. The system is highly sensitive and can detect interactions not revealed by other methods; even transient interactions may trigger transcription to produce a stable mRNA that can be repeatedly translated to yield the reporter protein. When a protein is identified that binds to an essential polypeptide, the two-hybrid system can be used in a binding assay to identify agents that inhibit binding and result in lack of a reporter signal.

Ligand binding assays known to the art may be used to search for agents that bind to the target protein. Without intending to be limiting, one such screening method to identify direct binding of test ligands to a target protein is described in Bowie et al. (U.S. Pat. No. 5,585, 277). This method relies on the principle that proteins generally exist as a mixture of folded and unfolded states, and continually alternate between the two states. When a test ligand binds to the folded form of a target protein (i.e., when the test ligand is a ligand of the target protein), the target protein molecule bound by the ligand remains in its folded state. Thus, the folded target protein is present to a greater extent in the presence of a test ligand which binds the target protein, than in the absence of a ligand. Binding of the ligand to the target protein can be determined by any method which distinguishes between the folded and unfolded state of the target protein. The function of the target protein need not be known in order for this assay to be performed.

Another method for identifying ligands for a target protein is described in Wieboldt et al., Anal. Chem., 69, 1683-1691 (1997). This technique screens combinatorial libraries of 20-30 agents at a time in solution phase for binding to the target protein. Agents that bind to the target protein are separated from other library components by centrifugal ultrafiltration. The specifically selected molecules that are retained on the filter are subsequently liberated from the target protein and analyzed by HPLC and pneumatically assisted electrospray (ion spray) ionization mass spectroscopy. This procedure selects library components with the greatest affinity for the target protein, and is particularly useful for small molecule libraries.

Another method allows the identification of ligands present in a sample using capillary electrophoresis (CE) (see Hughes et al., U.S. Pat. No. 5,783,397). The sample and the target protein are combined and resolved. The conditions of electrophoresis results in simultaneously fractionating the components present in the sample and screening for components that bind to the target molecule. This method is particularly useful for complex samples including, for instance, extracts of plants, animals, microbes, or portions thereof and chemical libraries produced by, for instance, combinatorial chemistry.

The agents identified by the initial screens are evaluated for their effect on survival of microbes, preferably H. influenzae, H. ducreyi, or H. aegyplius, more preferably, an H. influenzae. Agents that interfere with bacterial survival are expected to be capable of preventing the establishment of an infection or reversing the outcome of an infection once it is established. Agents may be bacteriocidal (i.e., an agent kills the microbe and prevents the replication of the microbe) or bacteriostatic (i.e., an agent reversibly prevents replication of the microbe). Preferably, the agent is bacteriocidal. Such agents will be useful to treat a subject infected with H. influenzae, H. ducreyi, or H. aegyptius, more preferably, an H. influenzae, or at risk of being infected by H. influenzae, H. ducreyi, or H. aegyptius, more preferably, an H. influenzae.

The identification of H. influenzae critical coding sequences, preferably, essential coding sequences, also provides for microorganisms exhibiting reduced virulence, which may be useful in vaccines. The term “vaccine” refers to a composition that, upon administration to a subject, will provide protection against H. influenzae, H. ducreyi, or H. aegyptius, more preferably, an H. influenzae. Administration of a vaccine to a subject will produce an immunological response to the H. influenzae and result in immunity. A vaccine is administered in an amount effective to result in some therapeutic benefit or effect so as to result in an immune response that inhibits or prevents an infection by H. influenzae in a subject, or so as to result in the production of antibodies to an H. influenzae.

Such microorganisms that can be used in a vaccine include H. influenzae, H. ducreyi, or H. aegyptius, more preferably, an H. influenzae, mutants containing a mutation in a coding sequence represented by any one of the coding sequence present in SEQ ID NO:11, 16, 26, 31, 46, 64, 69, 74, 79, 84, 89, 99, 104, 109, 114, 124, 129, 145, 165, 170, 180, 205, 210, 215, 220, 232, 237, 242, 247, 252, 357, 150, 185, 200, 267, 389-443, nucleotides 936-2429 of SEQ ID NO:225, nucleotides 2443-3809 of SEQ ID NO:225, or a coding sequence having structural similarity thereto. Optionally, an H. influenzae, H. ducreyi, or H. aegyptius, more preferably, an H. influenzae, includes more than one mutation. The reduced virulence of these organisms and their immunogenicity may be confirmed by administration to a subject. Animal models useful for evaluating H. influenzae virulence in a variety of conditions, including for example, otitis media in gerbils and chinchilla, are known in the art.

While it is possible for an avirulent microorganism of the invention to be administered alone, one or more of such mutant microorganisms are preferably administered in a vaccine composition containing a suitable adjuvant(s) and a pharmaceutically acceptable diluent(s) or carrier(s). The carrier(s) must be “acceptable” in the sense of being compatible with the avirulent microorganism of the invention and not deleterious to the subject to be immunized. Typically, the carriers will be water or saline which will be sterile and pyrogen free. The subject to be immunized is a subject needing protection from a disease caused by a virulent form of H. influenzae.

Any adjuvant known in the art may be used in the vaccine composition, including oil-based adjuvants such as Freund's Complete Adjuvant and Freund's Incomplete Adjuvant, mycolate-based adjuvants (e.g., trehalose dimycolate), bacteria lipopolysaccharide (LPS), peptidoglycans (i.e., mumins, mucopeptides, or glycoprotelns such as N-Opaca, muramyl dipeptide (MDP), or MDP analogs), proteoglycans (e.g., extracted from Klebsiela spp.), streptococcal preparations (e.g., OK432), the “Iscoms” of EP 109 942, EP 180 564 and EP 231 039, aluminum hydroxide, saponin, DEAE-dextran, neutral oils (such as miglyol), vegetable oils (such as arachis oil), liposomes, the Ribi adjuvant system (see, for example GB-A-2 189 141), or adjuvants available under the trade designation BIOSTIM (e.g., 01K2) and PLURONIC polyols. Recently, an alternative adjuvant consisting of extracts of Amycolata, a bacterial genus in the order Actinomycetales, has been described in U.S. Pat. No. 4,877,612. Additionally, proprietary adjuvant mixtures are commercially available. The adjuvant used will depend, in part, on the recipient organism. The amount of adjuvant to administer will depend on the type and size of animal. Optimal dosages may be readily determined by routine methods.

The vaccine compositions optionally may include pharmaceutically acceptable (i.e., sterile and non-toxic) liquid, semisolid, or solid diluents that serve as pharmaceutical vehicles, excipients, or media. Any diluent known in the art may be used. Exemplary diluents include, but are not limited to, polyoxyethylene sorbitan monolaurate, magnesium stearate, methyl-andpropylhydroxybenzoate, talc, alginates, starches, lactose, sucrose, dextrose, sorbitol, mannitol, gum acacia, calcium phosphate, mineral oil, cocoa butter, and oil of theobroma.

The vaccine compositions can be packaged in forms convenient for delivery. The compositions can be enclosed within a capsule, sachet, cachet, gelatin, paper or other container. These delivery forms are preferred when compatible with entry of the immunogenic composition into the recipient organism and, particularly, when the immunogenic composition is being delivered in unit dose form. The dosage units can be packaged, e.g., in tablets, capsules, suppositories or cachets.

The vaccine compositions may be introduced into the subject to be immunized by any conventional method including, e.g., by intravenous, intradermal, intramuscular, intramammary, intraperitoneal, or subcutaneous injection; by oral, sublingual, nasal, anal, vaginal, or transdermal delivery; or by surgical implantation, e.g., embedded under the splenic capsule or in the cornea. The treatment may consist of a single dose or a plurality of doses over a period of time. It will be appreciated that the vaccine of the invention may be useful in the fields of human medicine and veterinary medicine. Thus, the subject to be immunized may be a human or an animal, for example, cows, sheep, pigs, horses, dogs, cats, and poultry such as chickens, turkeys, ducks and geese.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Rapid Transposon Insertional Inactivation for Determining Essentiality of H. influenzae Coding Sequences

Materials and Methods

Bacterial strains and growth conditions: Haemophilus influenzae Rd was obtained from the American Type Culture Collection as ATCC 51907 and grown in brain-heart infusion (Becton Dickinson, Sparks, Md.) supplemented with 5% Fildes Enrichment (Becton Dickinson) (sBHI) at 37° C. in 5% CO₂.

Oligonucleotide synthesis: All of the oligonucleotides were synthesized by Genosys (The Woodlands, Tex.).

Target gene amplification and vector cloning: The target gene with flanking sequences was PCR amplified from 1 microgram (μg) of H. influenzae genomic DNA with the N/C primer set following the AMPLITAQ GOLD (Applied Biosystems, Foster City, Calif.) amplification protocol: 94° C. for 5 minutes (1 cycle); 94° C. for 30 seconds, 55° C. for 1 minute and 72° C. for 3 minutes (30 cycles); and 72° for 5 minutes (1 cycle). The resulting amplicon was cloned into the vector pCR2.1 following the enclosed directions of the Invritrogen cloning kit (Invitrogen, Carlsbad, Calif.) and transformed into competent E. coli InvαF′ cells purchased from Invitrogen. Colonies were screened by restriction analysis to confirm the presence K of the PCR insert. Vector DNA was isolated using columns purchased from Qiagen, Inc. (Valencia, Calif.).

New England Biolabs GPS-M Tn mutagenesis protocol: Transposon mutagenesis of the vector DNA was performed following the enclosed directions of the GPS-M Mutagenesis kit purchased from New England Biolabs (Beverly, Mass.). Briefly, 320 nanograms (ng) of vector DNA was added to 8 μl 10× GPS buffer, 4 μl GPS buffer 3 and dH₂O to 32 μl. After adding 4 μl of TnsABC transposase, the reaction was incubated for 10 minutes at 37° C. Four μl of Start Solution was then added and the reaction was incubated for 1 hour at 37° C. Following heat inactivation at 75° for 10 minutes, the reaction was phenol extracted, ethanol precipitated and resuspended in 84 μl 10 mM Tris-1 mM EDTA, pH 8.0 (TE). The transposon insertion sites were repaired by adding 4 μl DNA Polymerase I (E. coli) (New England Biolabs, Beverly, Mass.), 12 μl 10× PolA buffer (New England Biolabs), and 12 μl 300 μl dNTP mix to the resuspended vector and incubating for 15 min at room temperature. Four μl 30 mM ATP and 4 μl T4 DNA ligase were then added to ligate the transposon into the vector overnight at 16° C. The vector was then phenol extracted, ethanol precipitated and resuspended in 25 μl TE for transformation into H. influenzae.

Epicentre Transposon mutagenesis protocol: The target gene with flanking sequences was PCR amplified from 1 μg of H. influenzae genomic DNA with the N/C primer set following the GibcoBRL Platinum Taq amplification protocol: 94° C. for 5 minutes (1 cycle); 94° C. for 30 seconds, 55° C. for 1 minute and 72° C. for 3 minutes (30 cycles); and 72° C. for 5 minutes (1 cycle). Fifty microliters of the reaction was loaded onto a 1.2% agarose gel and the amplicon was isolated. Following the enclosed directions of the Epicentre (Madison, Wis.) EZ::TN <KAN-2> insertion kit, 100 nanograms (ng) of the PCR amplicon was mutagenized for 2 hours at 37° C. After heat inactivation of the transposase at 70° C. for 10 minutes, 20 μl of sterile water was then added and the reaction was passed through a Millipore Ultrafree column (Bedford, Mass.) to remove the enzyme. Gap repair was performed by adding 3 μl 10× E. coli PolA buffer, 3 μl 300 mM dNTP mix and 1.5 μl DNA polymerase I to the reaction and incubating for 15 minutes at room temperature.

Competent cell preparation and transformation of H. influenzae: Competent cells of H. influenzae were prepared following the protocol outlined by Barcak et al. (Methods in Enzymology, 204, 321-342 (1991)) for transformation using chemically defined M-IV medium. For DNA uptake and transformation a 1 milliliter (ml) vial of stored H. influenzae competent cells was pelleted for 5 minutes and resuspended in 1 ml of freshly prepared M-IV medium. One hundred nanograms of the mutagenized vector DNA was added to the cells. Following incubation for 30 minutes at 37° C., the cells were added to 5 mls sBHI medium and grown for 3 hours with shaking at 37° C. Cell aliquots of 100 μl, 250 μl, and 500 μl were plated onto sBHI plates supplemented with 30 μg/ml kanamycin and incubated overnight at 37° C. with 5% CO₂.

Colony screening: Individual isolates were initially screened for transposon inserts within the 5′ end of the target gene by PCR amplification using the K/Z primer set as follows: 94° C. for 5 minutes (1 cycle); 94° C. for 30 seconds, 55° C. for 1 minute and 72° C. for 1.5 minutes (25 cycles); and 72° C. for 5 minutes. Ten μl of each reaction was analyzed on a 1.2% agarose gel. If no inserts were detected, the plates were reincubated overnight and additional isolates were then screened targeting small colonies with reduced growth rates. PCR screening for transposon inserts within the entire target amplicon was performed using the N/C primer set as follows: 94° C. for 5 minutes (1 cycle); 94° C. for 30 seconds, 55° C. for 1 minute and 72° C. for 3 minutes (30 cycles); and 72° C. for 5 minutes.

PCR sequencing: PCR products synthesized with either the K/Z or N/C primer pairs were isolated using the Qiagen MinElute columns. One hundred nanograms of PCR product, 100 ng of Epicentre Kan-2 FP-1 transposon sequencing primer ACCTACAACAAAGCTCTCATCAACC (SEQ ID NO:499) and sterile water to a final volume of 12 μl were added to a single tube and the nucleotide sequence determined.

Results

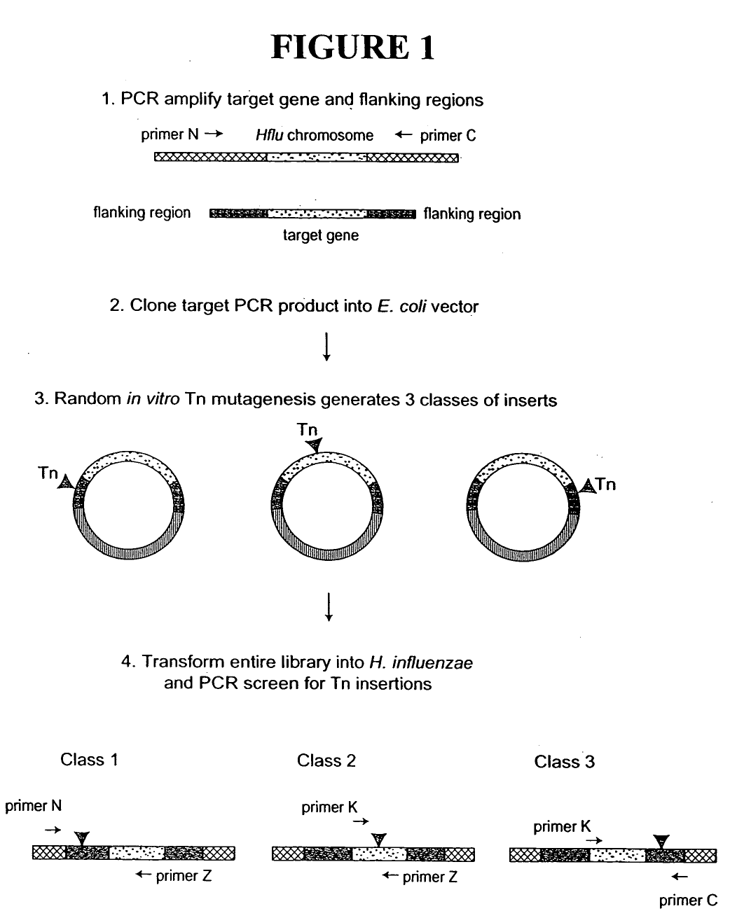

The initial overall strategy for targeted gene disruption is outlined in FIG. 1. Primers approximately 1000 base pairs (bp) upstream (N) and downstream (C) of the target gene are designed to amplify the target gene and flanking sequences from Haemophilus genomic DNA. The resulting PCR product is cloned into the E. coli pCR2.1 vector, which cannot replicate in H. influenzae. The vector is then subjected to in vitro transposon mutagenesis, generating a library containing three classes of inserts that are of interest: those with transposon inserts upstream or downstream of the target gene, and those with inserts in the target gene itself. Transformation of the library into H. influenzae allows the vector to recombine into the chromosome, replacing the chromosomal copy of the target gene with the mutagenized PCR product. Plating the cells on kanamycin selects for colonies that have successfully undergone recombination. The resulting colonies are then screened by PCR within the 5′ end of the target gene for the presence of a transposon. The first 300-400 base pairs of the 5′ end is defined as the “essential” region, and primers K and Z are designed to amplify this region. PCR product bands corresponding to the target K/Z fragment do not contain an insert. PCR bands increasing in size by 1200 bp contain a transposon insert. If a transposon insert is present within the essential region, then the gene is determined to be non-essential. If no transposon inserts are found within the essential region, then the upstream and downstream flanking sequences are screened by PCR with primer pair N/C to identify transposon inserts within the flanking regions as confirmation that mutagenesis occurred. If no inserts are found within the essential region but are found within the flanking regions, then the gene is determined to be essential.

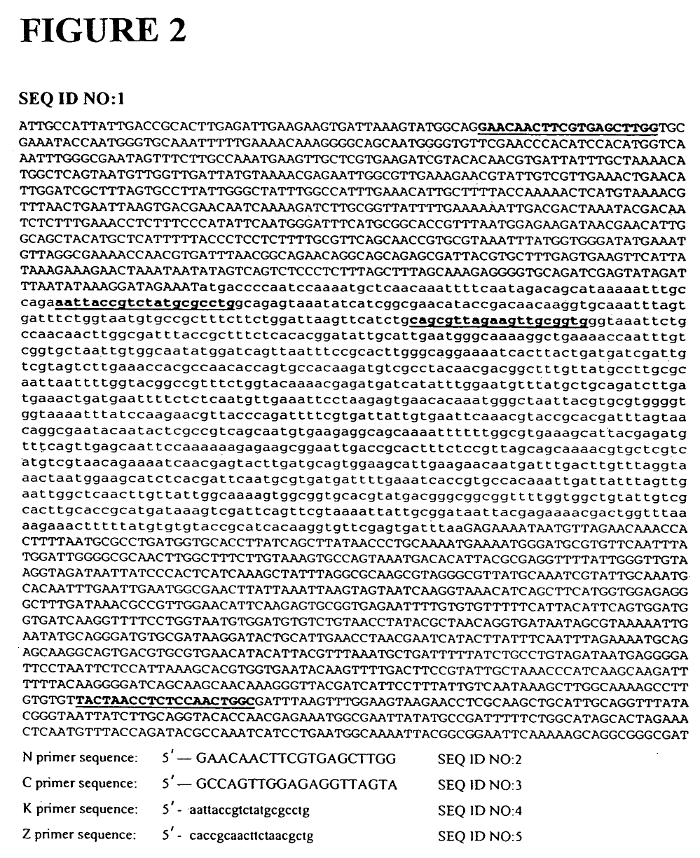

Inactivation of a non-essential gene: We began our assessment of transposon mutagenesis by selecting a known non-essential H. influenzae gene for targeted insertional inactivation. The galK gene (HI0819) and flanking sequences (FIG. 2) were PCR amplified from H. influenzae genomic DNA and cloned into the pCR2.1 vector. Isolated vector DNA was mutagenized in vitro with the GPS-M Mutagenesis kit, transformed into H. influenzae, and plated onto sBHI supplemented with kanamycin. Nineteen colonies were screened by PCR with primer pair K/Z for the presence of transposon inserts within the essential 5′ region. Six of the transformants examined contained transposon inserts; therefore, galK was determined to be non-essential.

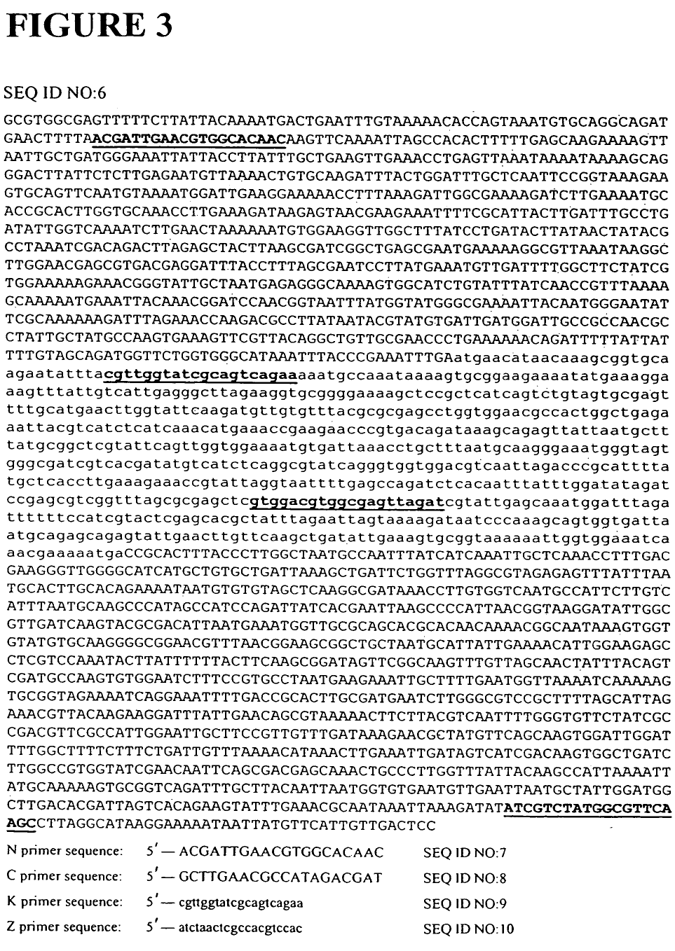

Identification of an essential gene: Having shown that a known non-essential gene could be insertionally inactivated by this method, we then chose a gene expected to be essential for mutagenesis. The H. influenzae tmk gene (HI0456) and flanking sequences (FIG. 3) were PCR amplified from H. influenzae genomic DNA and cloned into the pCR2.1 vector. Following GPS-M in vitro mutagenesis, the vector was transformed into H. influenzae. Recombinants selected on kanamycin plates were then screened by PCR for the presence of transposon inserts within the tmk 5′ essential region using primer pair K/Z. No inserts were-found within any of the sixty-four recombinants screened. These colonies were then screened with the N/C primer pair to identify inserts within the upstream or downstream flanking regions. Fourteen transposon inserts were found upstream of the tmk gene, but none were found downstream of the tmk gene. Since no inserts were found in the essential region but were found upstream, tmk was determined to be essential. Since no inserts were found in the downstream holB gene, this suggested that this gene may also be essential.

E. coli confirmation of an essential gene: One advantage to using the vector system for mutagenesis is that confirmation of the H. influenzae results can be obtained using E. coli. The mutagenized pCR2.1 vector containing the tmk gene was transformed into E. coli. Transformants were screened by PCR to identify three individual colonies, one containing a vector with a transposon insert in the upstream flanking region, one containing a transposon insert in the downstream flanking region, and one containing a transposon insert in the tmk gene itself. Plasmid DNA was isolated from all three individual colonies and separately transformed into H. influenzae. Kanamycin selected recombinants were then screened by PCR for the presence of the original transposon insert. None were found within the tmk gene or the downstream holB gene, even though each transformed vector contained a transposon insert within these genes. This confirmed that tmk and holB gene were essential. Inserts were again found within the upstream region suggesting that this gene may not be essential. These results are in agreement with the paper published by Akerley et al. (Proc. Nat. Acad. Sci. USA, 95, 8927-8932 (1998)) in which it was determined that tmk and holB are essential, whereas the upstream conserved hypothetical protein is not.

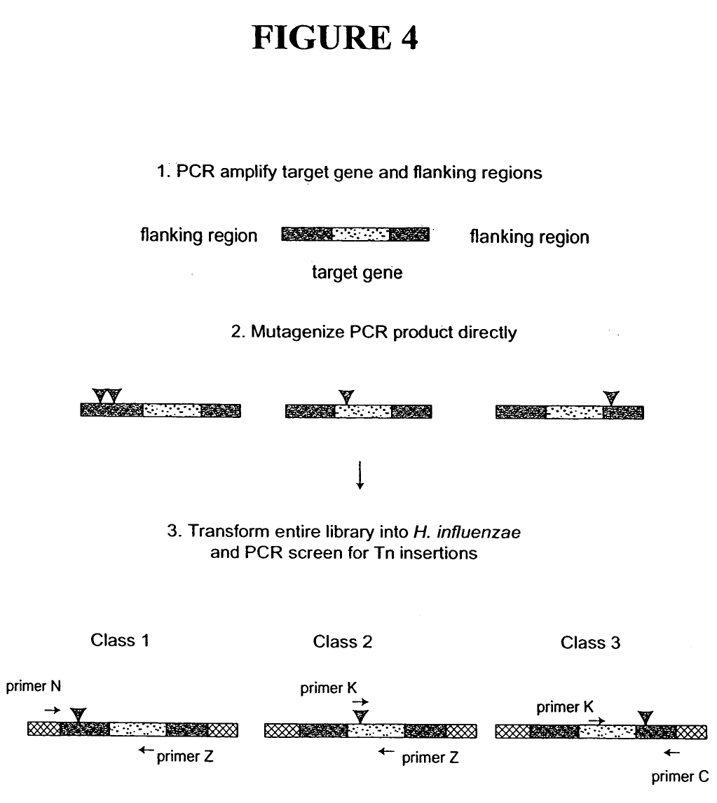

Direct mutagenesis of the target gene PCR product: There are several inherent difficulties associated with the vector method for insertional inactivation. In vitro mutagenesis results in transposons inserting within the entire vector sequence, not just the target region. This causes high backgrounds and increases the number of recombinants that have to be screened for insertions within the target region. Cloning can be time-consuming and not all PCR products can be cloned into the pCR2.1 vector. The cloned target region may also contain errors from the PCR amplification. To overcome these difficulties, we chose to evaluate if we could perform in vitro mutagenesis on the target PCR product itself and subsequently transform it directly into H. influenzae since H. influenzae has a natural single-stranded DNA uptake system. This protocol is outlined in FIG. 4. The galK gene and flanking sequences were PCR amplified from H. influenzae genomic DNA and mutagenized in vitro with the Epicenter EZ::Tn <Kan-2> transposon. The mutagenized PCR product was then transformed into H. influenzae. Recombinants selected on kanamycin plates were then screened with the K/Z primer pair for inserts within the galK 5′ essential region. Of the 48 colonies screened 6 contained an insert, confirming that galK is not essential. Since these results were identical to the results obtained with the vector method and were achieved more rapidly, the direct mutagenesis of a target gene PCR product with the EZ::Tn <Kan-2> transposon is now the standard methodology by which we evaluate gene essentiality in H. influenzae.

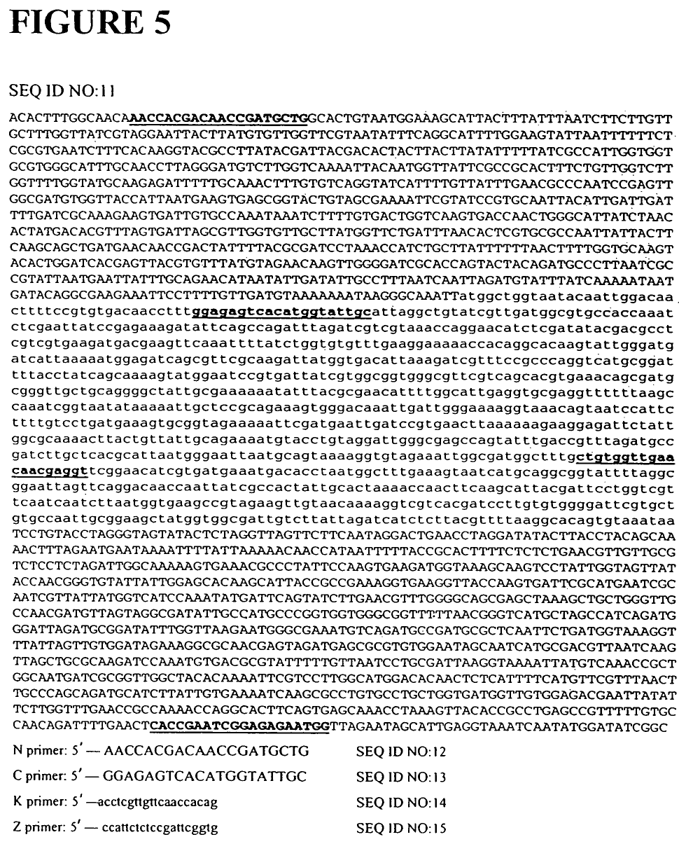

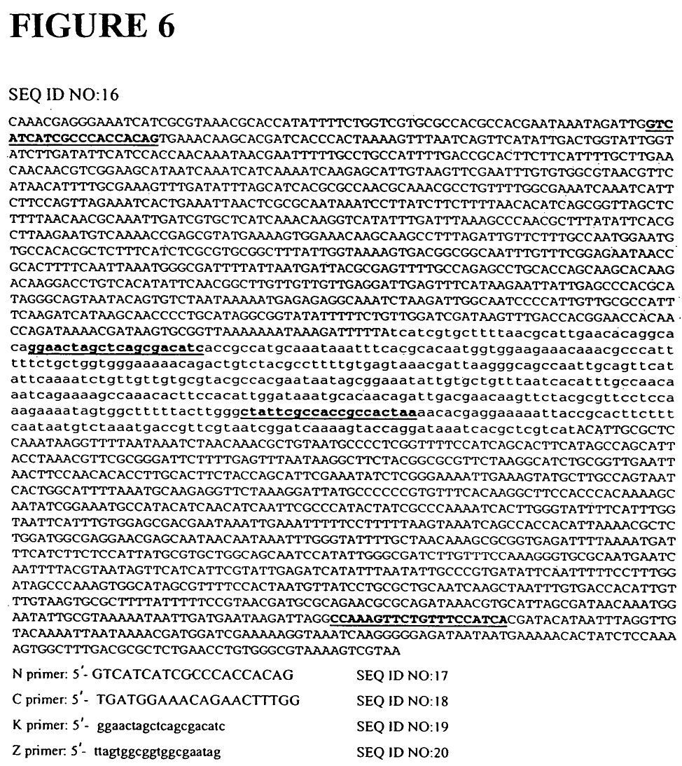

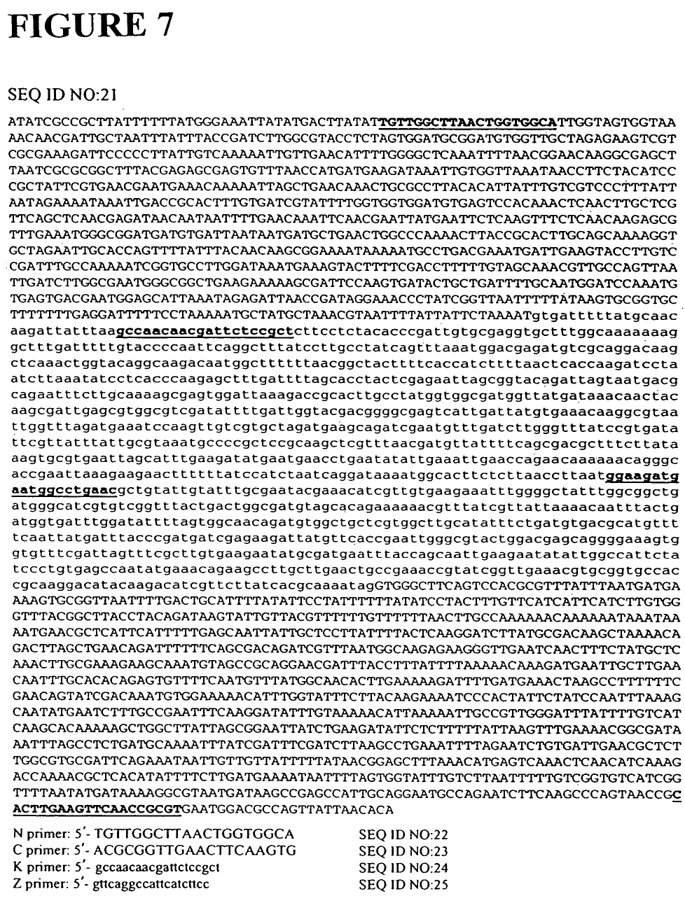

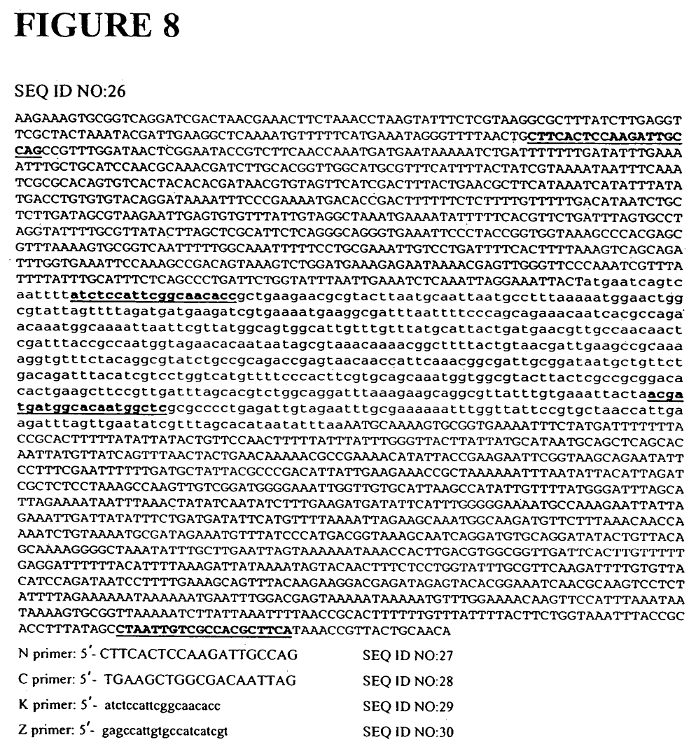

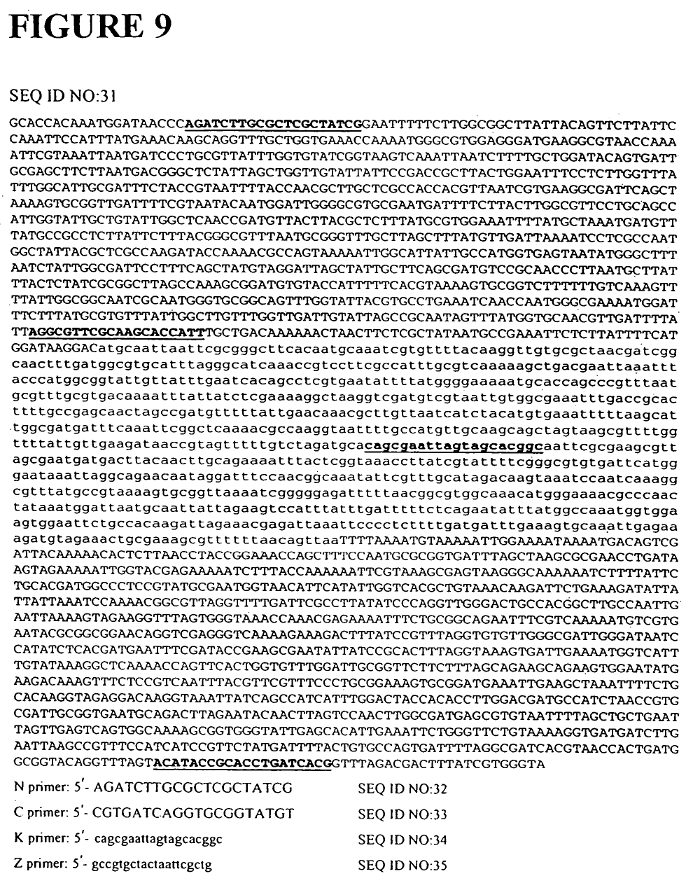

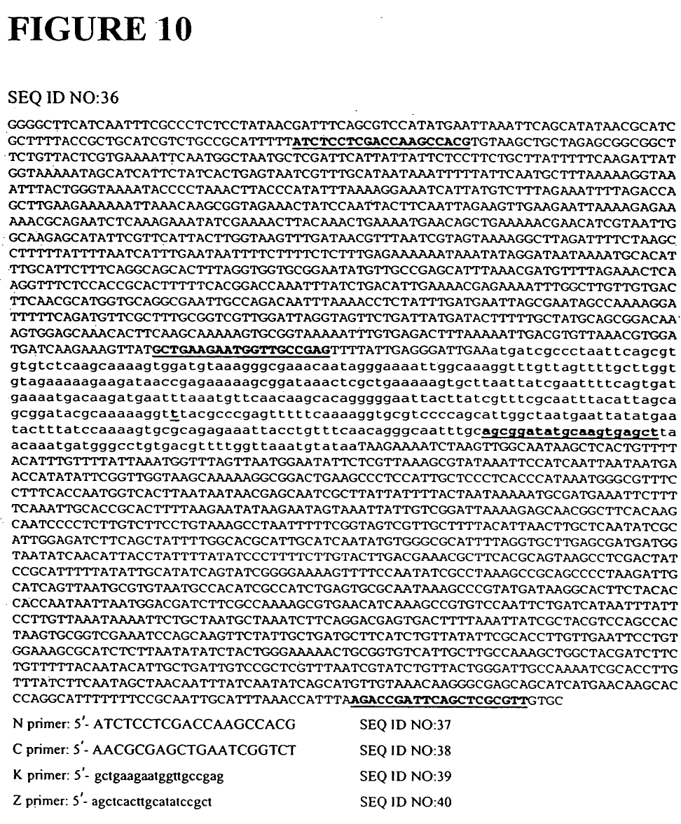

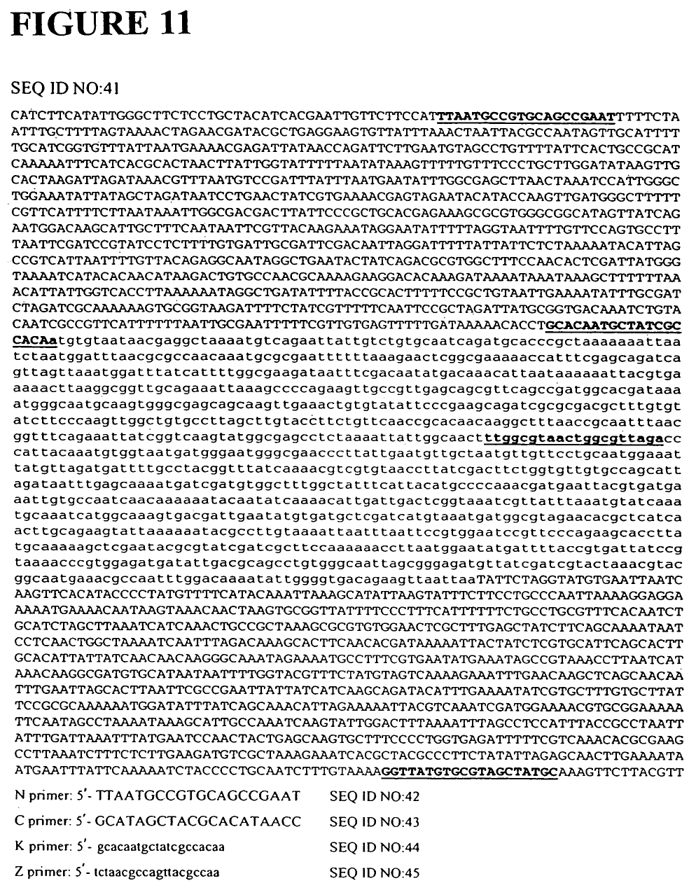

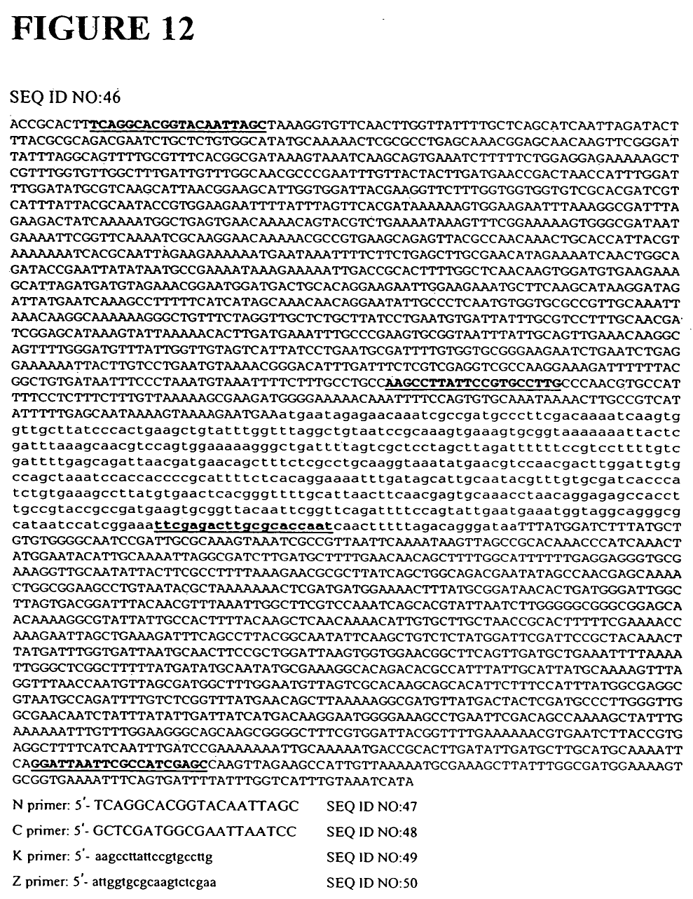

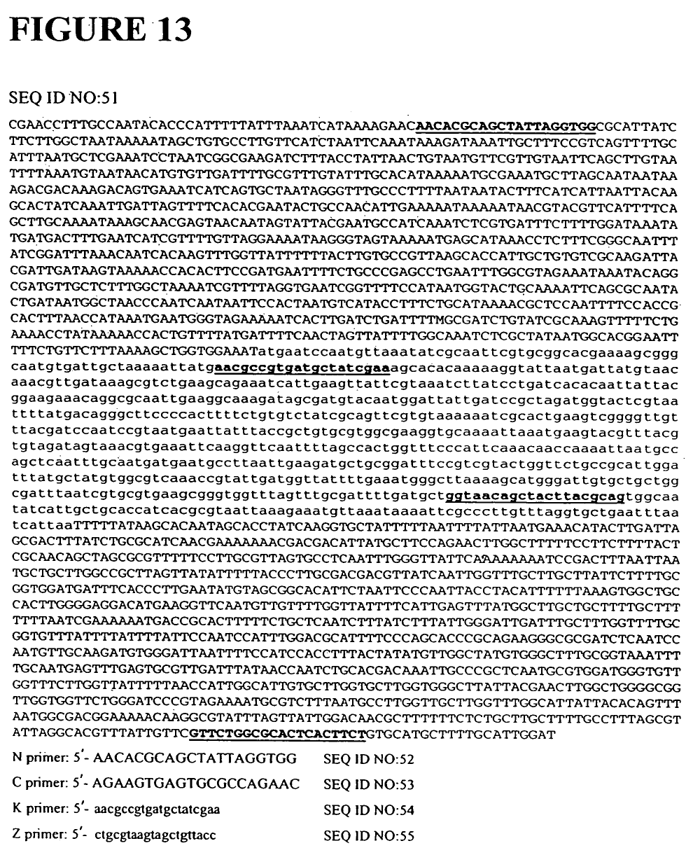

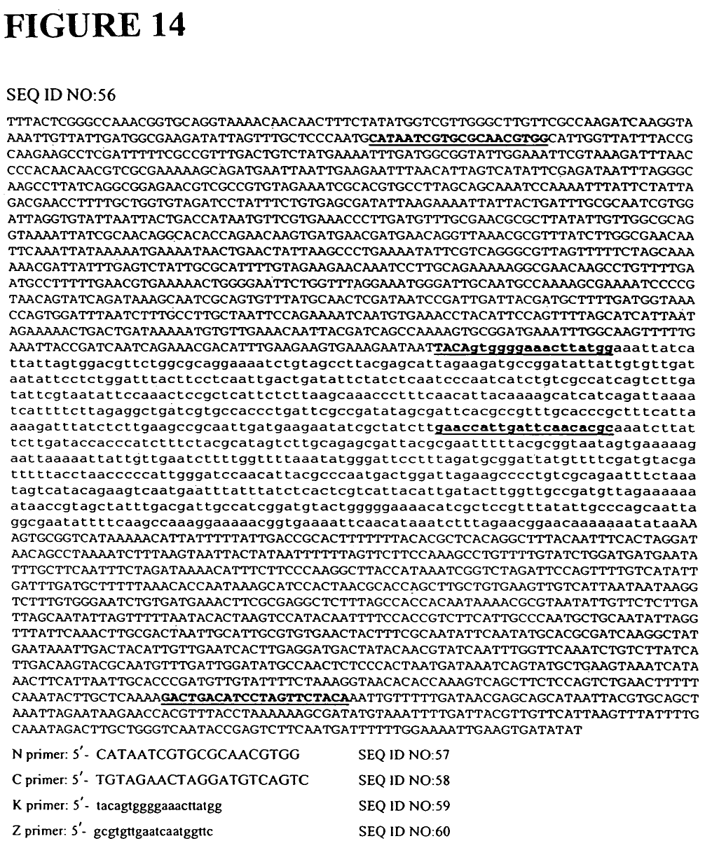

Evaluation of potential antibacterial targets: A list of potential new antibacterial targets was assembled for insertional inactivation to determine their essentiality in H. influenzae. The list included aroC (chorismate synthase), coaD (phosphopantetheine adenylyltransferase), rhlB (an ATP-dependent RN A helicase), ribB (3,4-dihydroxy-2-butone 4-phosphate synthase), ribF (riboflavin kinase), the conserved hypothetical proteins yihZ and yfgB. All of the gene sequences, flanking regions and PCR primer pairs are shown in FIGS. 5-14. For each gene, the PCR product was synthesized, mutagenized with the EZ::Tn <Kan-2> transposon, and. transformed directly into H. influenzae. Recombinants selected on kanamycin plates were screened with their essential region K/Z primer pair. The results are summarized in Table 1.

TABLE 1
Summary of the evaluation of the potential new antibiotic targets for
essentiality in H. influenzae. Five genes were found to be
essential: aroC, coaD, ribB, ribF and yrdC.

| | SEQ ID | | SEQ ID NO: of | | | |
|---|---|---|---|---|---|---|
| Gene | NO:¹ | Locus² | polypeptide | Putative identification² | Inactivated | No. Of Inserts |
| aroC | 11 | HI0196 | 282 | chorismate biosynthesis | No | 0/96 |
| coaD | 16 | HI0651 | 283 | panthothenate biosynthesis | No | 0/96 |
| rhlB | 21 | HI0892 | 284 | riboflavin biosynthesis | Yes | 21/48 |
| ribB | 26 | HI0764 | 285 | riboflavin biosynthesis | No | 0/96 |
| ribF | 31 | HI0963 | 286 | RNA helicase | No | 0/96 |
| yihZ | 36 | HI0670 | 287 | conserved hypothetical | Yes | 7/16 |
| | | | | protein | | |
| yfgB | 41 | HI0365 | 288 | conserved hypothetical | Yes | 13/48 |
| | | | | protein | | |
| yrdC | 46 | HI0656 | 289 | conserved hypothetical | No | 0/96 |
| | | | | protein | | |
| suhB | 51 | HI0937 | 290 | extragenic suppressor | Yes | 6/48 |
| yhbJ | 56 | HI1146 | 291 | conserved hypothetical | Yes | 20/48 |
| | | | | protein | | |

¹Refers to the coding sequence present at each SEQ ID NO.
²Information obtained through the TIGR Haemophilus influenzae KW20 locus search page (www.tigr.org/tigr-scripts/CMR2/LocusNameSearch.spl?db=ghi).

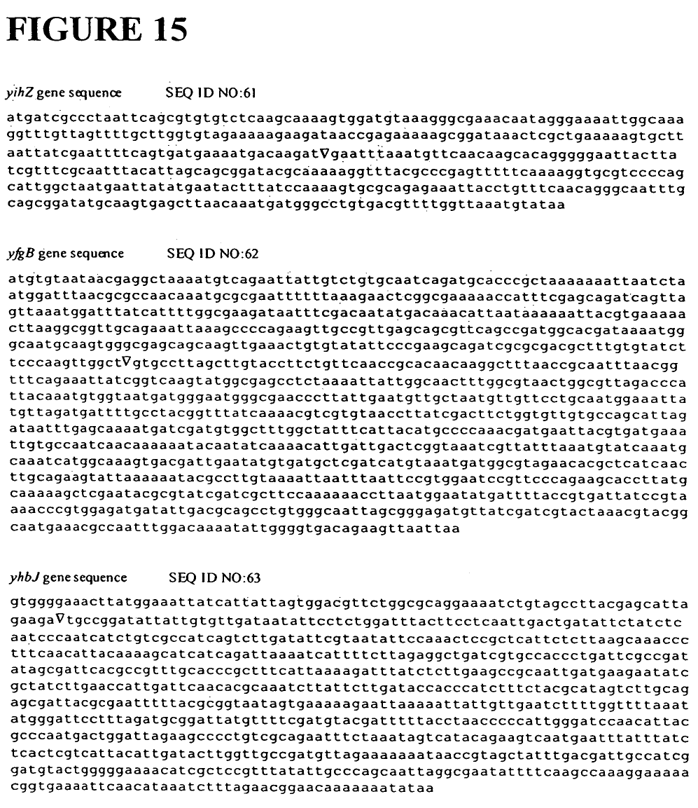

Inserts were found in rhlB, yihZ, yfgB, suhB and yhbJ. Since the K primer sequence occurred upstream of the ATG start codon for yihZ, yfgB, and yhbJ, one recombinant containing an insert was sequenced with a transposon primer to determine the exact location of the insert within each gene. All three transposons inserted within the 5′ essential region (FIG. 15); therefore, yihZ, yfgB, and yhbJ are non-essential. The K primers for rhlB and suhB were within the gene coding region, so rhlB and suhB were also non-essential. No inserts were found within aroC, coaD, ribB, ribF and yrdC, so their recombinants were then re-screened with their N/C primer pair to identify inserts within the flanking regions. Inserts were found in the flanking regions of all five genes; therefore, aroC, coaD, ribB, ribF and yrdC were determined to be essential.

### Example 2

### Rapid Transposon Insertional Inactivation for Determining Essentiality of Additional H. influenzae Coding Sequences

This demonstrates the use of rapid transposon insertional inactivation to evaluate 15 additional H. influenzae coding sequences.

Materials and Methods

The materials and methods used are described in Example 1.

Results

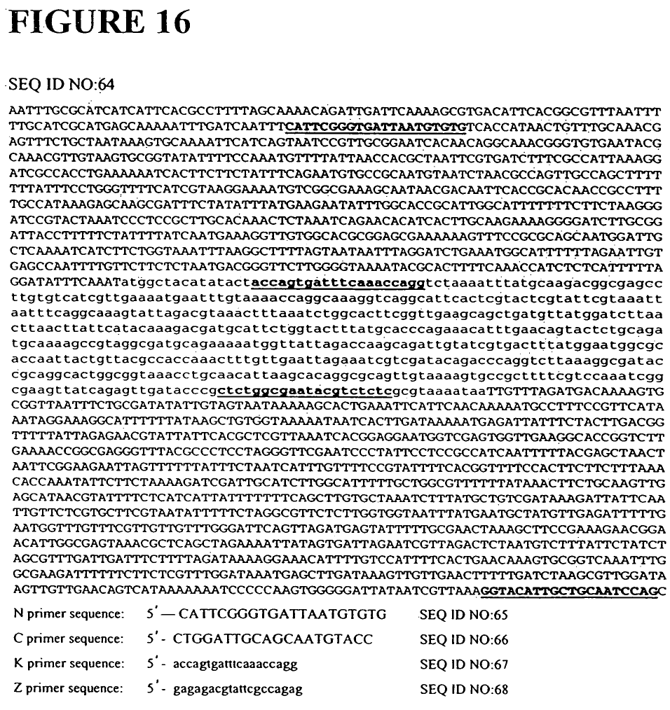

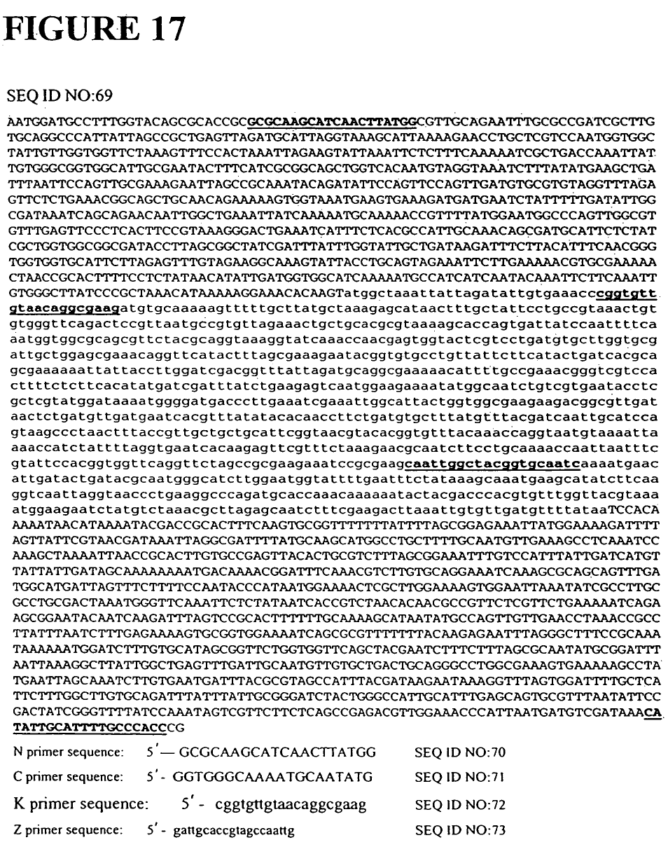

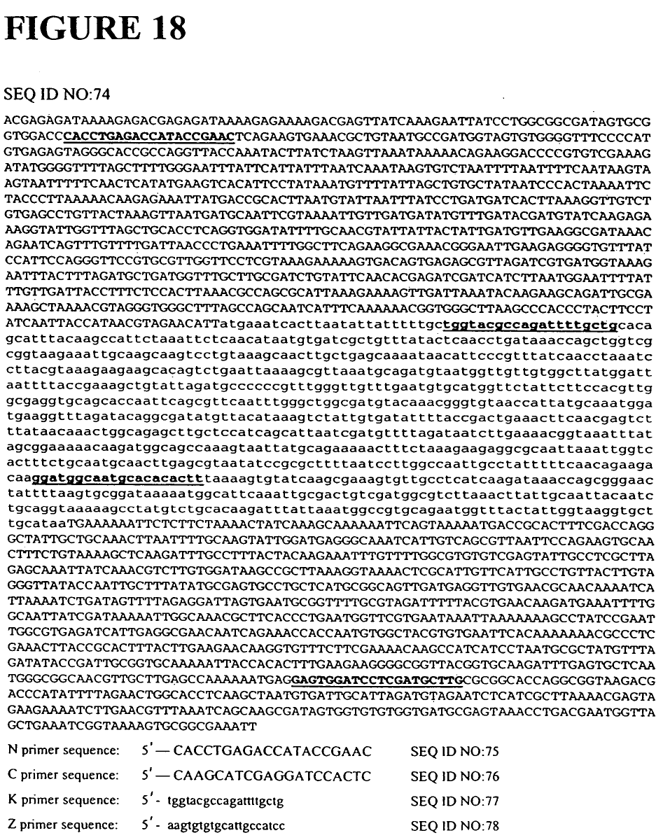

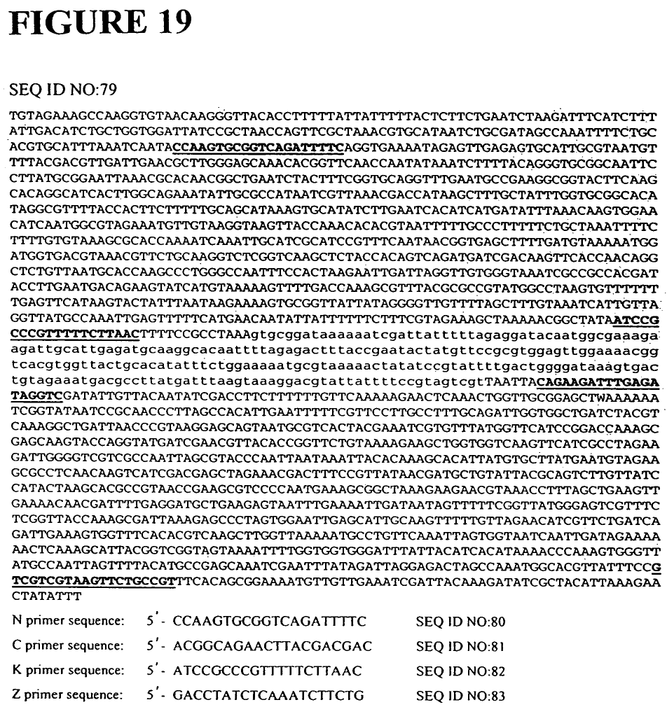

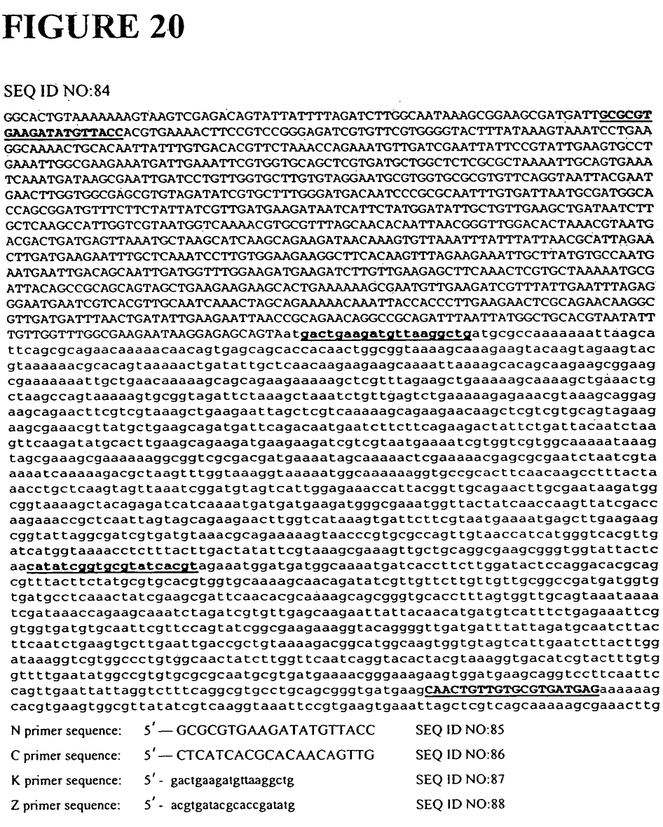

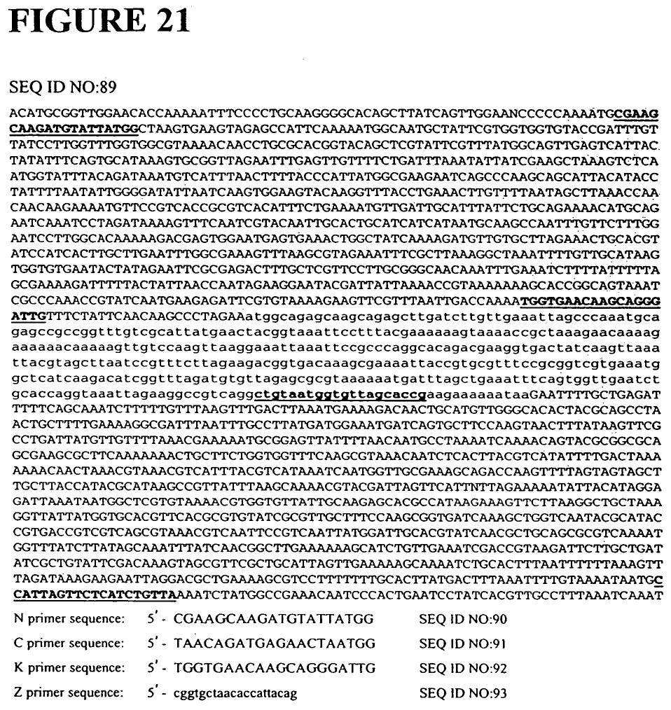

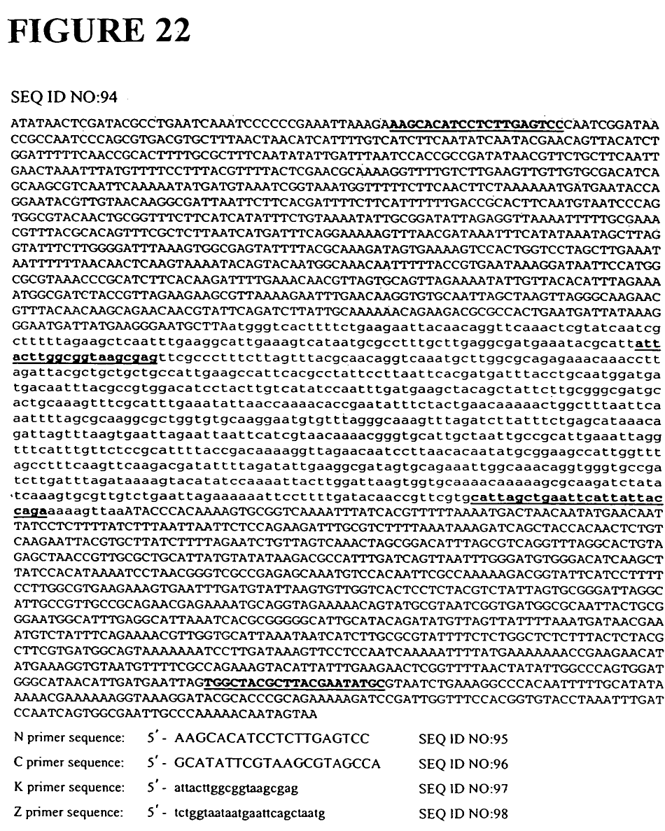

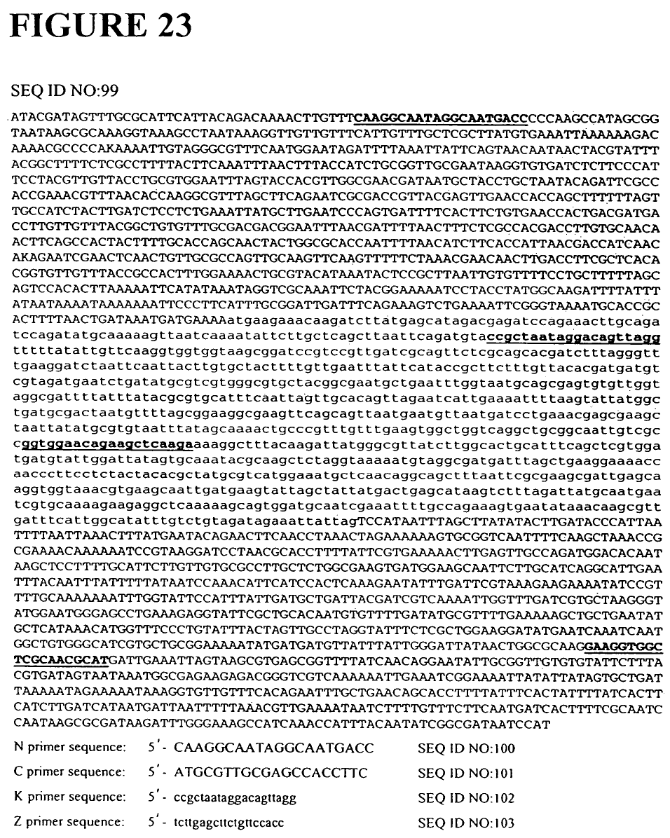

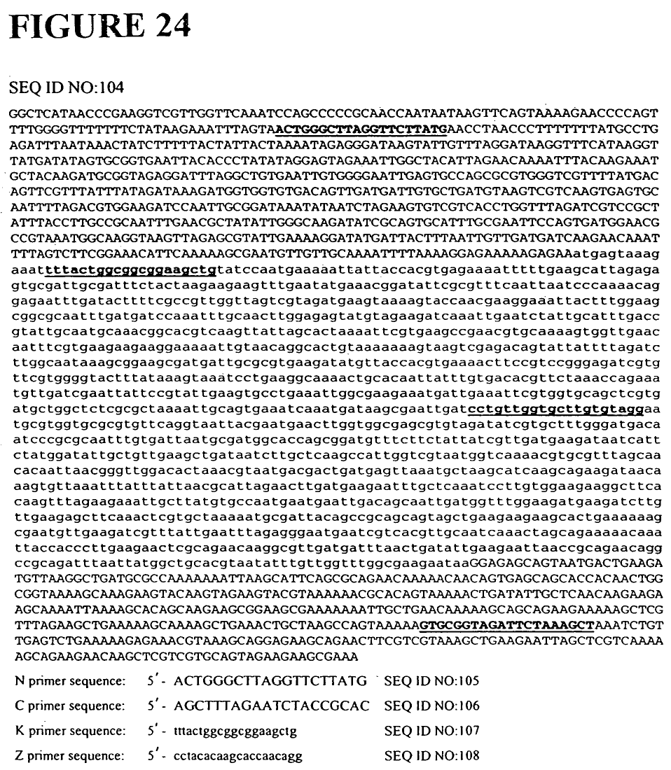

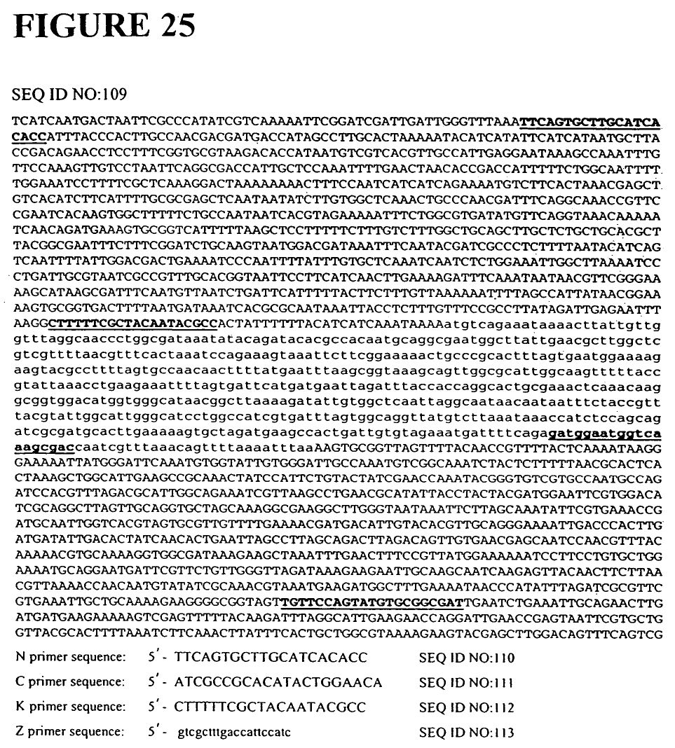

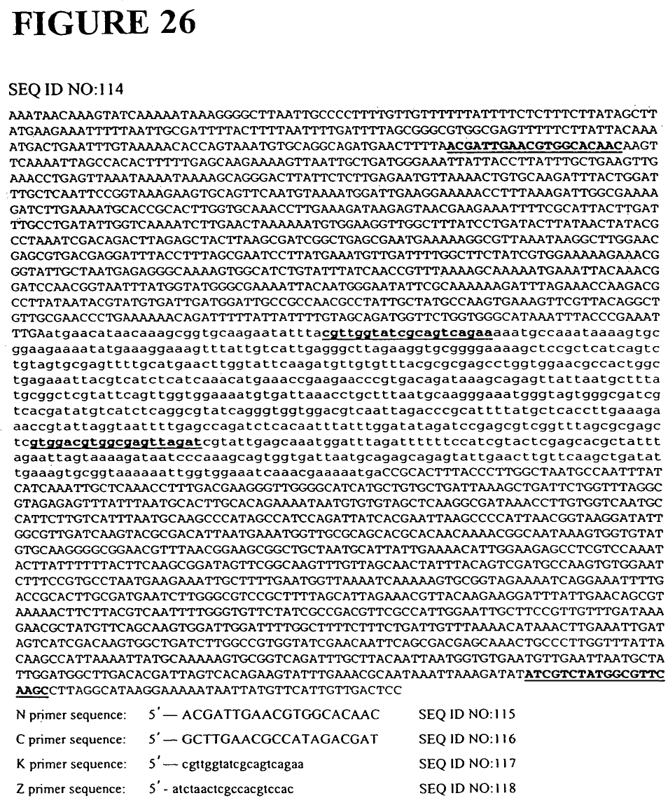

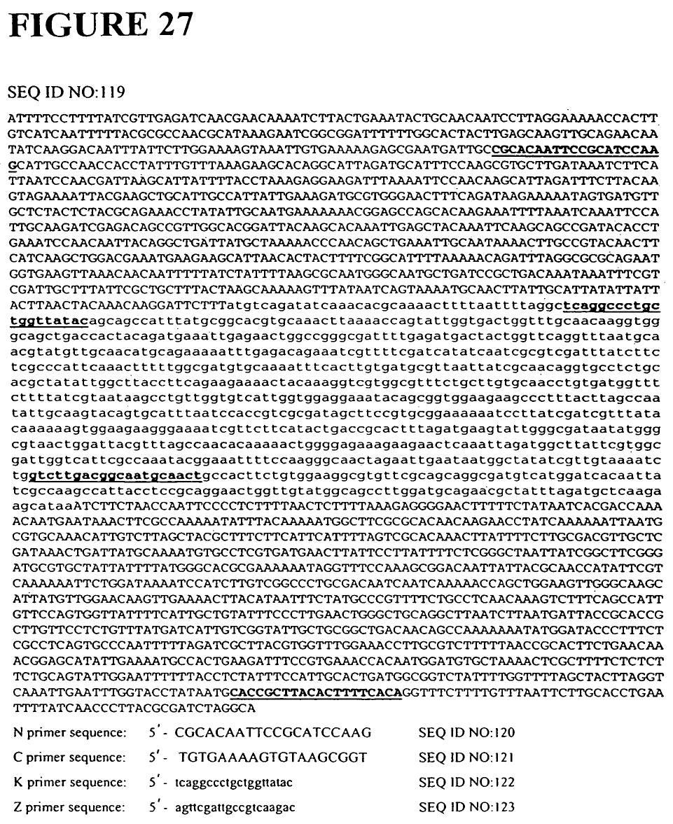

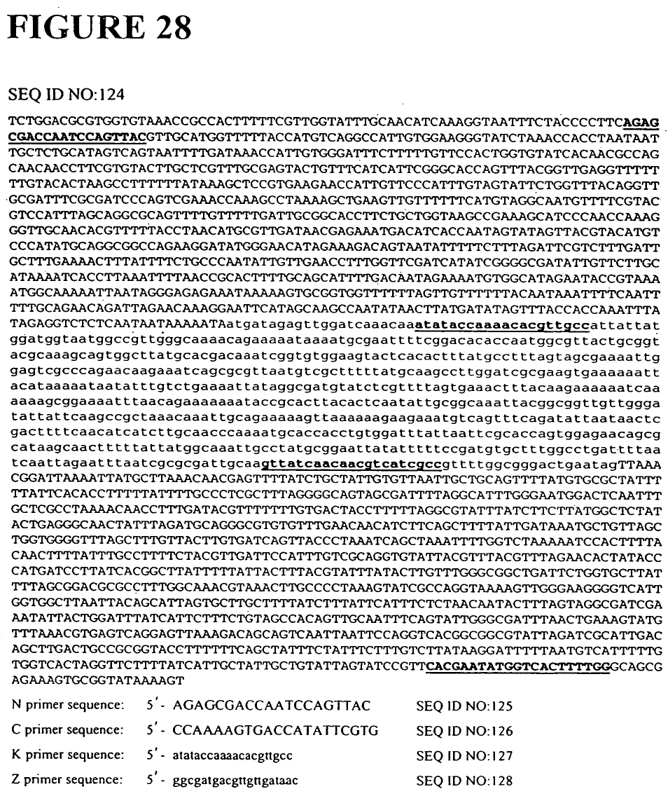

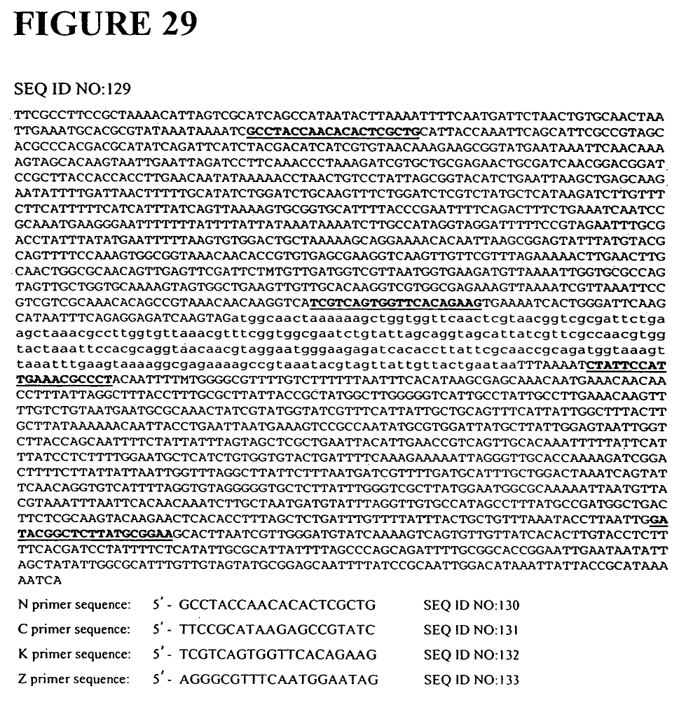

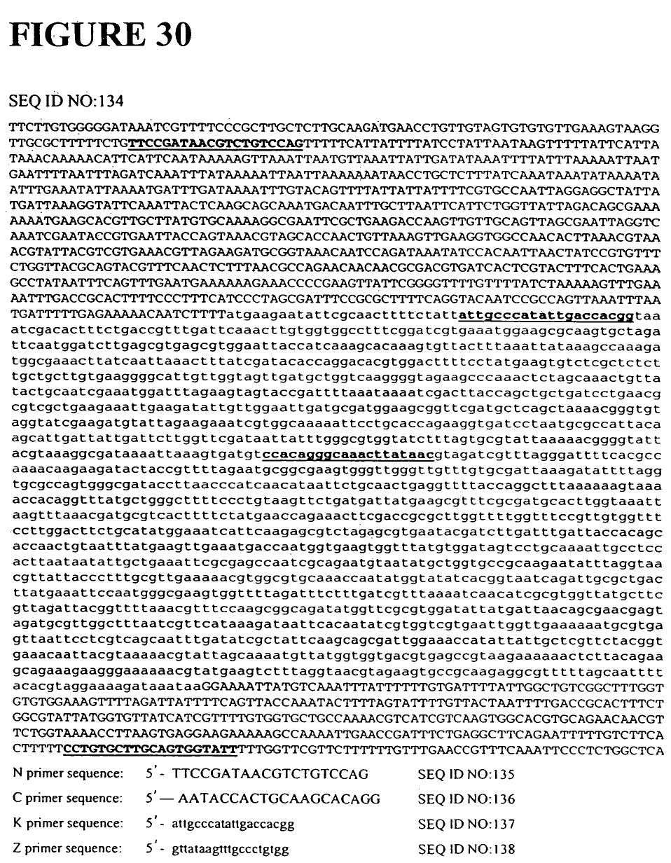

The following genes were subjected to insertional inactivation: efp (elongation factor P), fba (fructose-biphosphate aldolase), fmt (methionyl-tRNA formyltransferase), IF-1 (translation initiation factor 1), IF-2 (translation initiation factor 2), IF-3 (translation initiation factor 3), ispA (geranyl transferase), ispB (octaprenyl-diphosphate synthase), nusA (N utilization substance protein A), tmk (thymidylate kinase), trxB (thioredoxin reductase), pth (peptidyl-tRNA hydrolase), uppS (undecaprenyl pyrophosphate synthetase), L27 (ribosomal protein rpL27) and lepA (GTP-binding membrane protein). All of the gene sequences, flanking regions and PCR primer pairs are shown in FIGS. 16-30. For each gene, the PCR product was synthesized, mutagenized with the EZ::Tn <Kan-2> transposon, and transformed directly into H. influenzae. Kanamycin selected recombinants were then screened with their essential region K/Z primer pair. The results are summarized in Table 2.

TABLE 2
Summary of the evaluation of additional potential new
antibiotic targets for essentiality in H. influenzae.
Nine genes were determined to be essential: fba, fmt,
IF1, IF-2, IF-3, nusA, pth, tmk, and uppS.

| | | | SEQ ID | | | |
|---|---|---|---|---|---|---|
| | SEQ | | NO: of | | | |
| | ID | | poly- | Putative | | No. of |
| Gene | NO:¹ | Locus² | peptide | identification² | Insertion³ | inserts |
| efp | 64 | HI0328 | 292 | Translation | + | 33/96* |
| fba | 69 | HI0524 | 293 | Intermediary | − | 0/96 |
| | | | | metabolism | | |
| fmt | 74 | HI0623 | 294 | Translation | − | 0/96 |
| IF-1 | 79 | HI0548 | 295 | Translation | − | 0/96 |
| IF-2 | 84 | HI1248 | 296 | Translation | − | 0/96 |
| IF-3 | 89 | HI1318 | 297 | Translation | − | 0/96 |
| ispA | 94 | HI1438 | 298 | Cell wall | + | 8/48 |
| ispB | 99 | HI0881 | 299 | Quinone | + | 2/48* |
| | | | | synthesis | | |
| nusA | 104 | HI1283 | 300 | Transcription | − | 0/96 |
| pth | 109 | HI0394 | 301 | Protein synthesis | − | 0/96 |
| tmk | 114 | HI0456 | 302 | DNA synthesis | − | 0/96 |
| trxB | 119 | HI1158 | 303 | Redox | + | 20/48 |
| upps | 124 | HI0920 | 304 | Cell wall | − | 0/96 |
| L27 | 129 | HI0879 | 305 | Translation | plus | 13/96* |
| lepA | 134 | HI0016 | 306 | Translation | plus | 11/48 |

*Exhibit a slow growth phenotype when insertion is present in the coding sequence.
¹Refers to the coding sequence present at each SEQ ID NO.
²Information obtained through the TIGR Haemophilus influenzae KW20 locus search page (www.tigr.org/tigr-scripts/CMR2/LocusNameSearch.spl?db=ghi).
³“+”, gene was inactivated; “−”, gene was not inactivated.

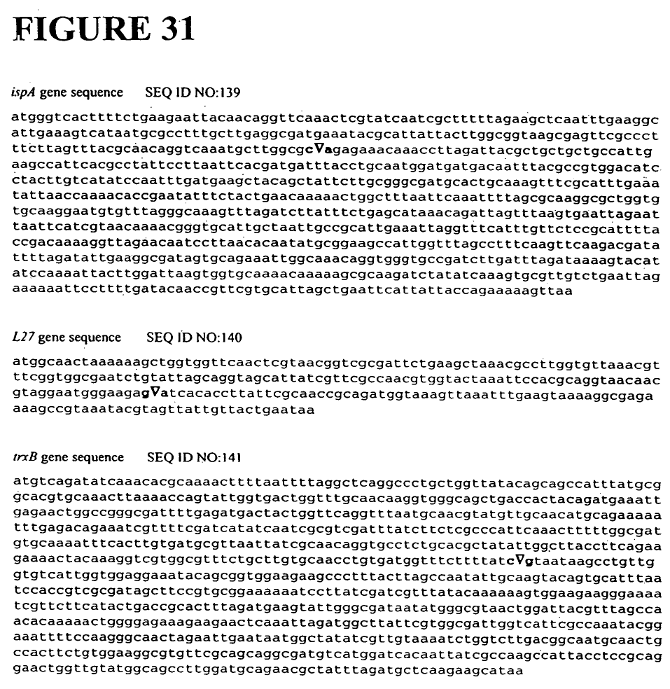

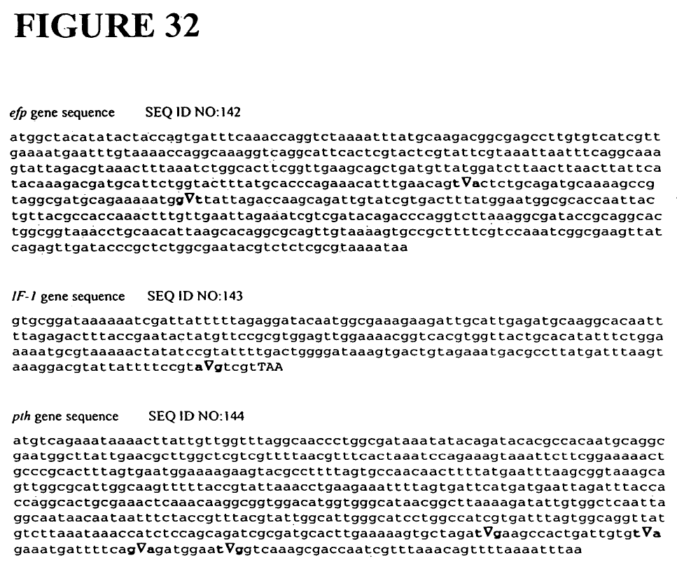

Inserts were found in efp, IF-1, ispA, ispB, pth, trxB, L27 and lepA. Since the K primer sequence occurred upstream of the ATG start codon for ispA, L27 and trxB, one recombinant containing an insert was sequenced with a transposon primer to determine the exact location of the insert within each gene. All three transposons inserted within the 5′ essential region (FIG. 31); therefore, ispA, L27 and trxB are non-essential. The Z primer sequence encompassed the C-terminal region of efp, IF-1 and pth. Since there are reports that transposons can insert within the C-terminal region of essential genes (Akerley et al., Proc. Nat. Acad. Sci. USA, 95, 8927-8932 (1998)), several recombinants containing inserts in these three genes were sequenced to determine the exact location of the inserts. Two independent insertions within efp were mapped to the middle of the gene (FIG. 32); therefore, efp is not essential. However all four insertions within the pth gene mapped to the C-terminal end of pth, and the insertion within the IF-1 gene also mapped to the C-terminal end of IF-1 (FIG. 32). Therefore, we conclude that pth and IF-1 are essential. The K/Z primer pairs for ispB and lepA are within the 5′ coding region, so these genes are non-essential. No inserts were found within the essential region of fba, fmt, IF-2, IF-3, nusA, tmk, and uppS. Recombinant colonies were then re-screened with their N/C primer pair to identify inserts within the flanking regions. Inserts were found in the flanking regions of all seven genes; therefore, fba, fmt, IF-2, IF-3, nusA, tmk, and uppS were determined to be essential.

Discussion

The unexpected results with several of the genes illustrates the importance of determining gene essentiality in a variety of organisms when selecting potential targets for broad spectrum antibiotic development. In this study fmt (methionyl-tRNA formyltransferase) is essential in H. influenzae whereas it is not essential in E. coli (Mazel et al., EMBO J., 13, 914-923 (1994)). Likewise efp (elongation factor P) and ispB (octaprenyl-diphosphate synthase) are essential in E. coli (Aoki et al., J. Biol. Chem., 272, 32254-32259 (1997); Okada et al., J. Bacteriol., 179, 3058-3060 (1997)), but not essential in H. influenzae. These results show that even organisms within broad categories such as gram-negatives or gram-positives can require different genes for bacterial survival.

The design of the K/Z primer pair is of critical importance when determining essentiality based on PCR results. If the K primer is designed upstream of the ATG start codon for the practical considerations of GC content and secondary structure, there is the possibility that transposons can insert prior to the. ATG. K/Z screening simply detects transposon inserts anywhere within the K/Z fragment. It does not determine the position of the insertion; therefore, those clones containing insertions downstream of the K primer but upstream of the ATG (not within the target gene) would yield the same PCR product as those clones containing insertions within the actual gene sequence. Hence, it is critical to sequence map clones containing insertions if the K primer is upstream of the ATG codon. Likewise sequence mapping of potential insertions is critical for those genes whose Z primer encompasses the C-terminal end of the gene, since it is possible for essential genes to contain insertions within this region. This most commonly occurs with small genes (<400 bp in length). Our results with pth and IF-1 illustrate, this point. Based on their K/Z PCR product results, both genes were initially determined to be non-essential. However, only after sequence mapping of the inserted clones was it determined that since all of the detected insertions occurred within the C-terminal region of each gene, pth and IF-1 were really essential.

### Example 3

### Rapid Transposon Insertional Inactivation for Determining Essentiality of H. influenzae Coding Sequences Associated with Cell Wall Biosynthesis

Materials and Methods

The materials and methods used are described in Example 1.

Results

There are 28 genes associated with cell wall biosynthesis in H. influenzae. The genes directly involved in the biosynthesis of peptidoglycan include glmS (glucosamine-fructose-6P-transferase), glmU (UDP-N-acetylglucosamine pyrophosphorylase), murZ (UDP-NAcGlu 1-carboxyvinyltransferase), murB (UDP-N-acetylenolpyruvoylglucosamine reductase), murC (UDP-N-acetylmuramate-alanine ligase), murD (UDP-N-acetylmuramoylalanine-D-glutamate ligase), murE (UDP-N-acetylmuramyl-tripeptide synthetase), murF (UDP-MurNAc-pentapeptide synthetase), murG (UDP-N-acetylglucosamine-N-acetylmuramyl-pyrophosphoryl UDP N-acetylglucosamine transferase), murI (glutamate racemase), mraY (phospho-N-acetylmuramoyl-pentapeptide-transferase E), alr (alanine racemase), and ddlB (D-alanine-D-alanine ligase). The seven penicillin-binding proteins include ponA (penicillin-binding protein 1A), ponB (penicillin-binding protein 1B), pbp2 (penicillin-binding protein 2), ftsI (penicillin-binding protein 3), dacB (penicillin-binding protein 4), dacA (penicillin-binding protein 5), and pbpG (penicillin-binding protein 7, putative). The remaining genes are those associated with cell wall biosynthesis. They include amiB (N-acetylmuramoyl-L alanine amidase), glnA (glutamine synthase), lpp (lipoprotein PCP precursor), mepA (penicillin-insensitive murein peptidase), mtgA (peptidoglycan transglycosylase), nagA (N-acetylglucosamine-6P-deacetylase), pal (outer membrane protein p6 precursor), and slt (soluble lytic murein transglycosylase).

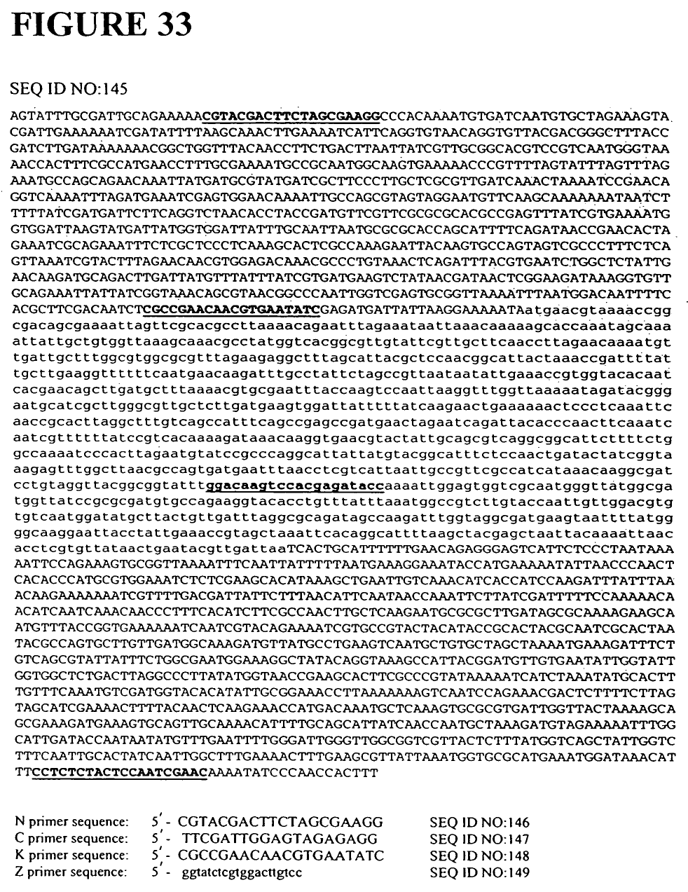

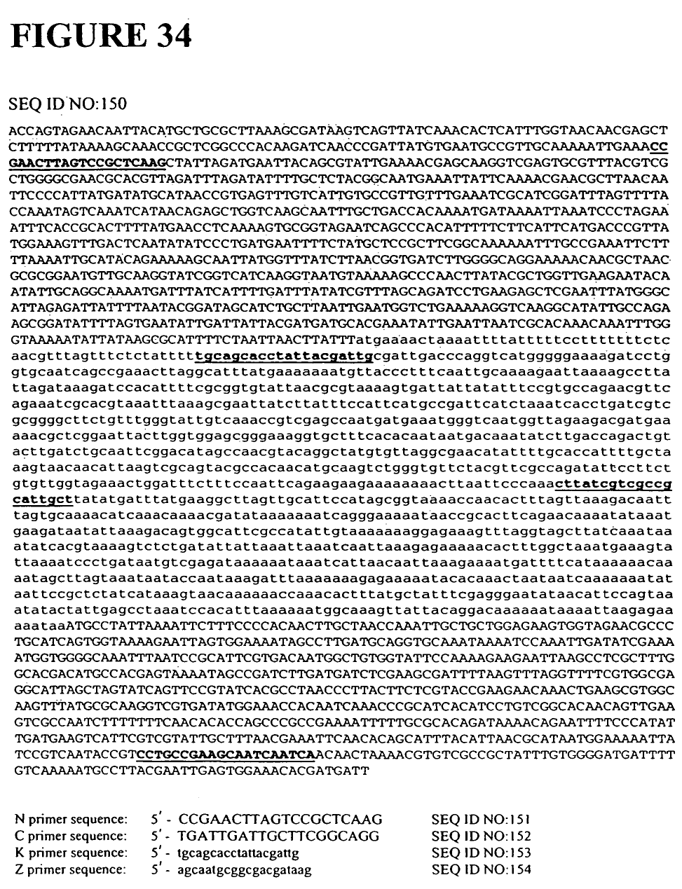

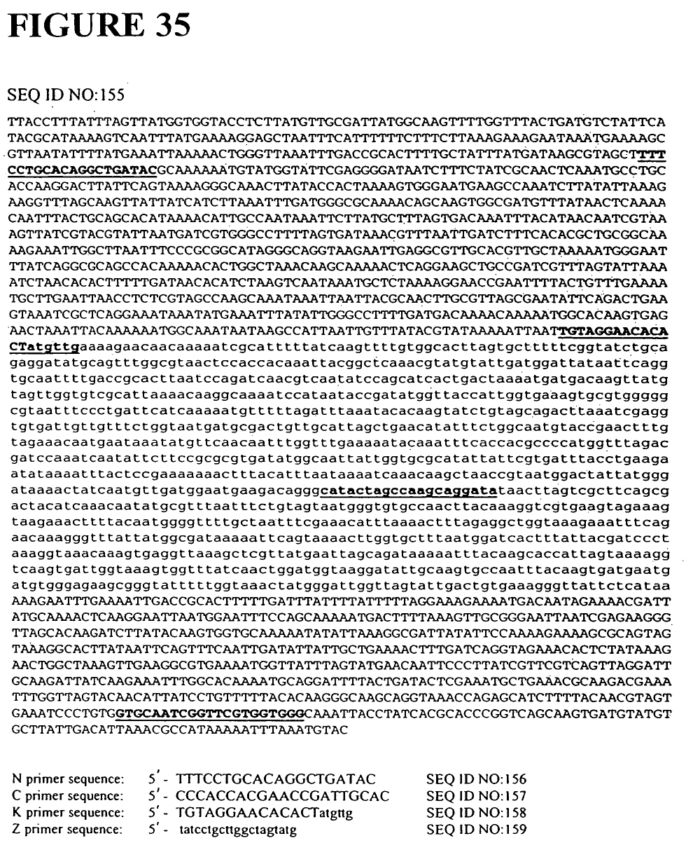

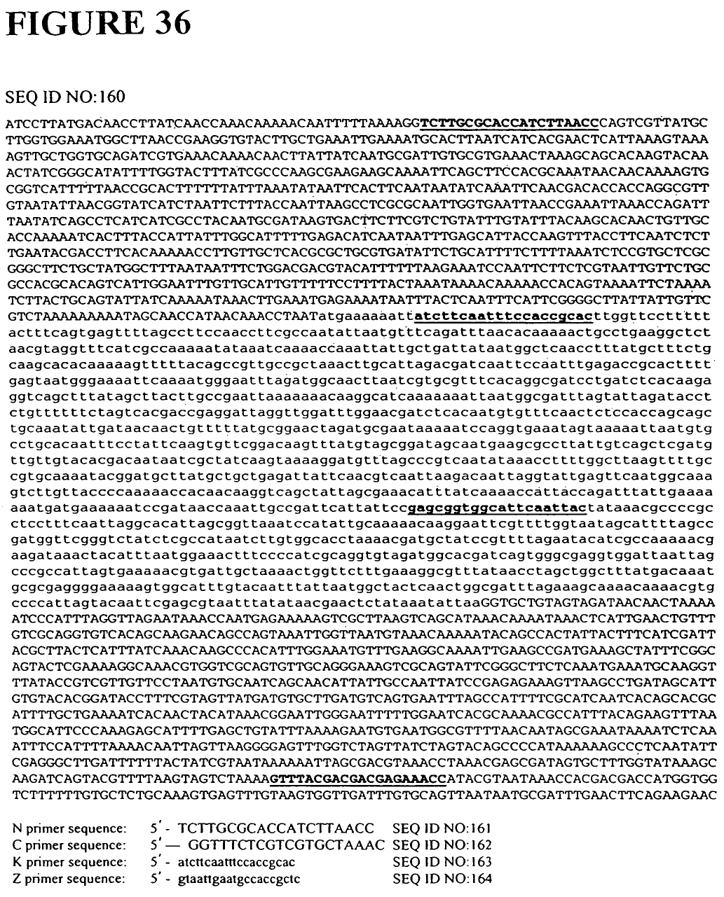

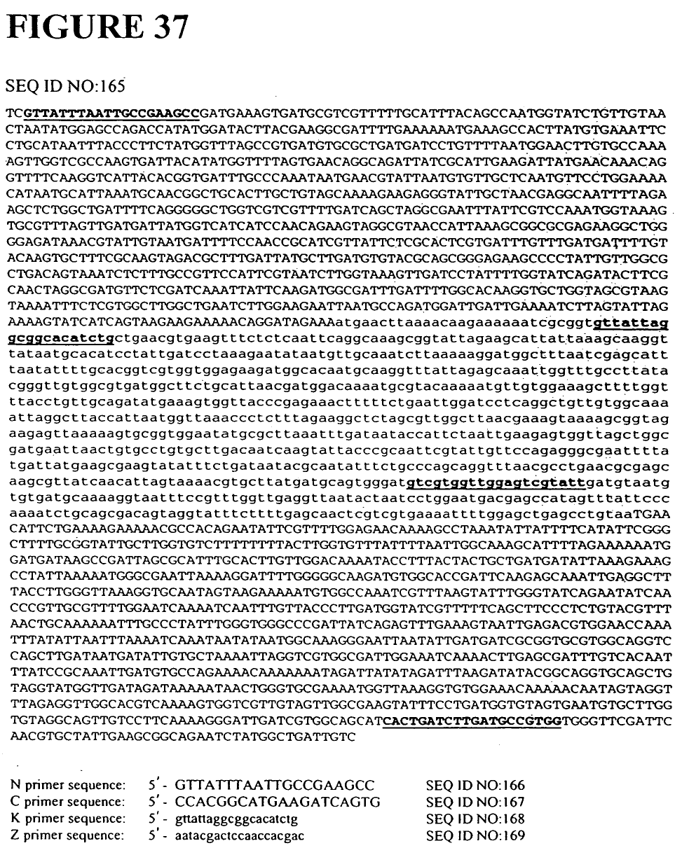

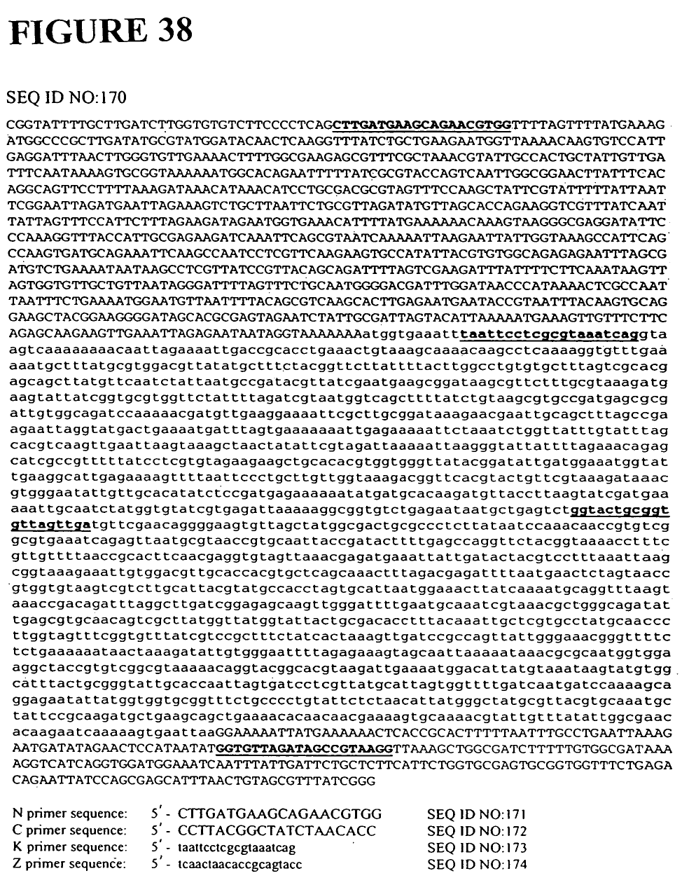

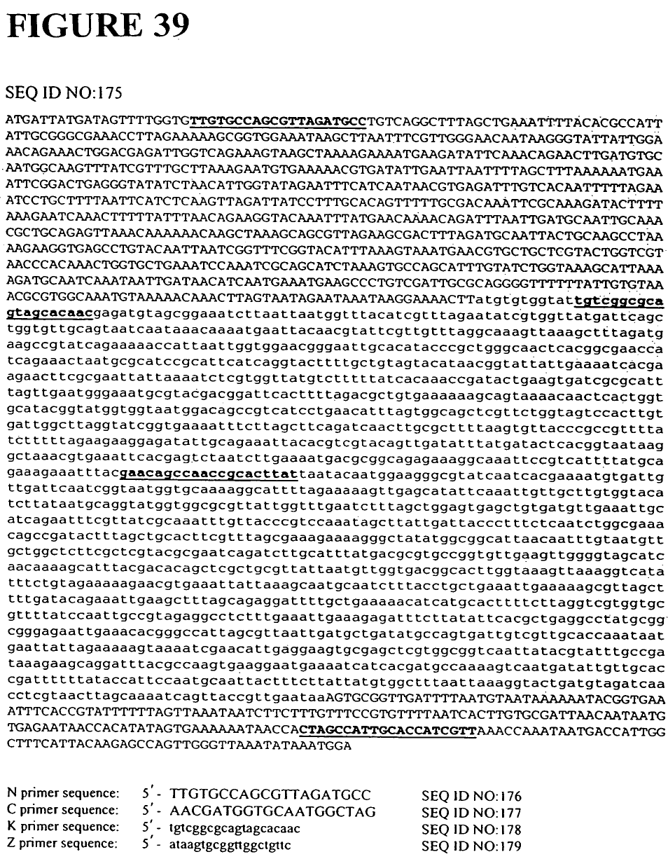

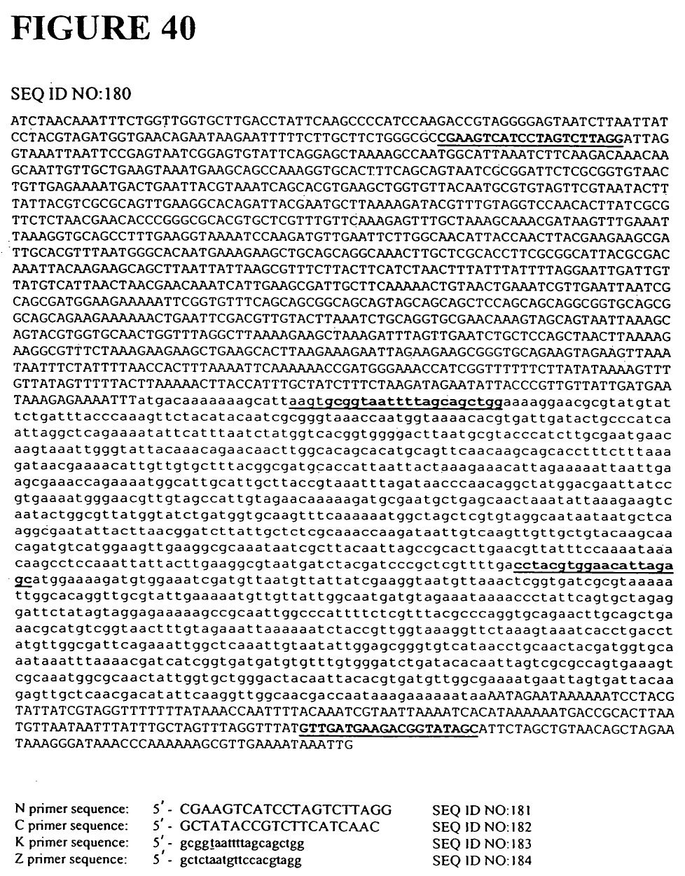

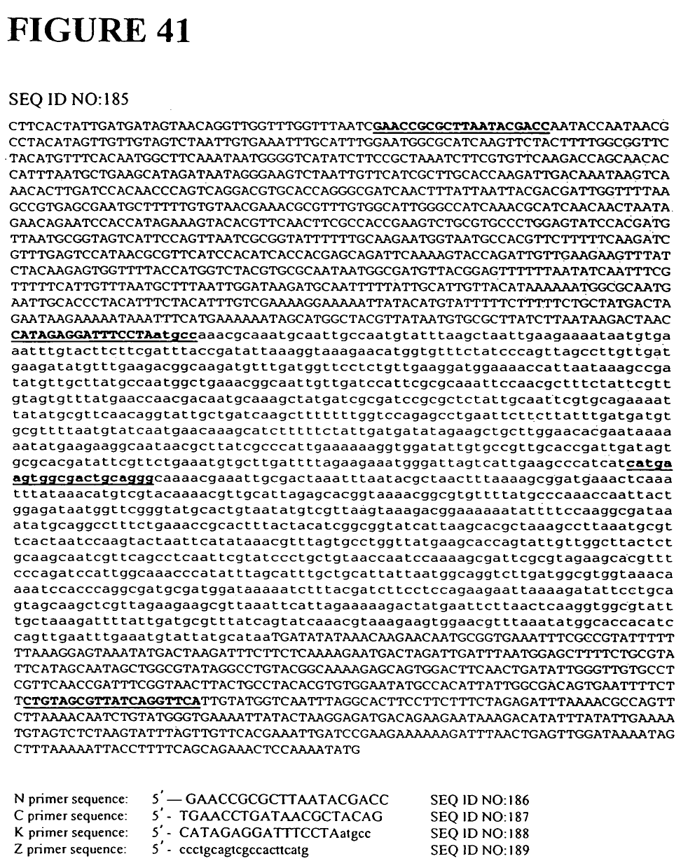

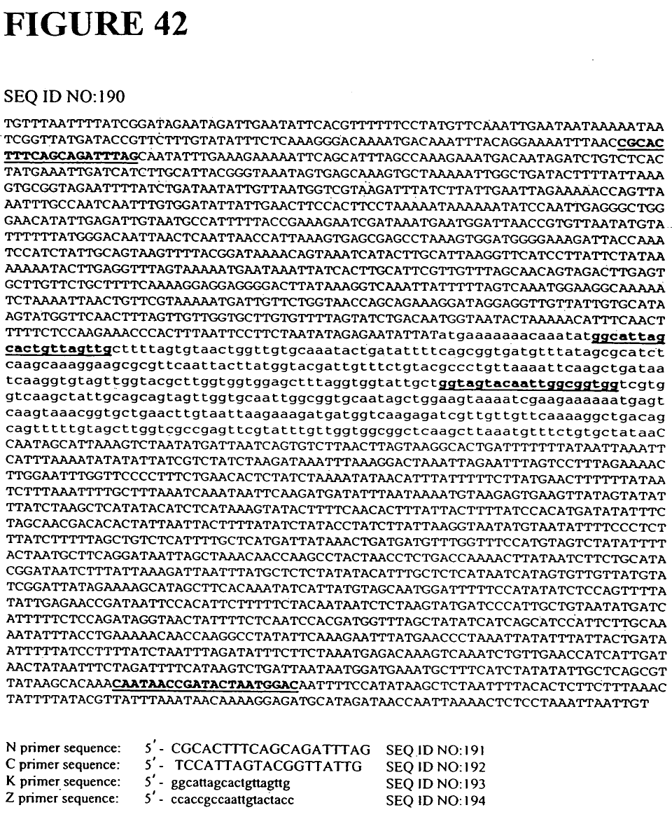

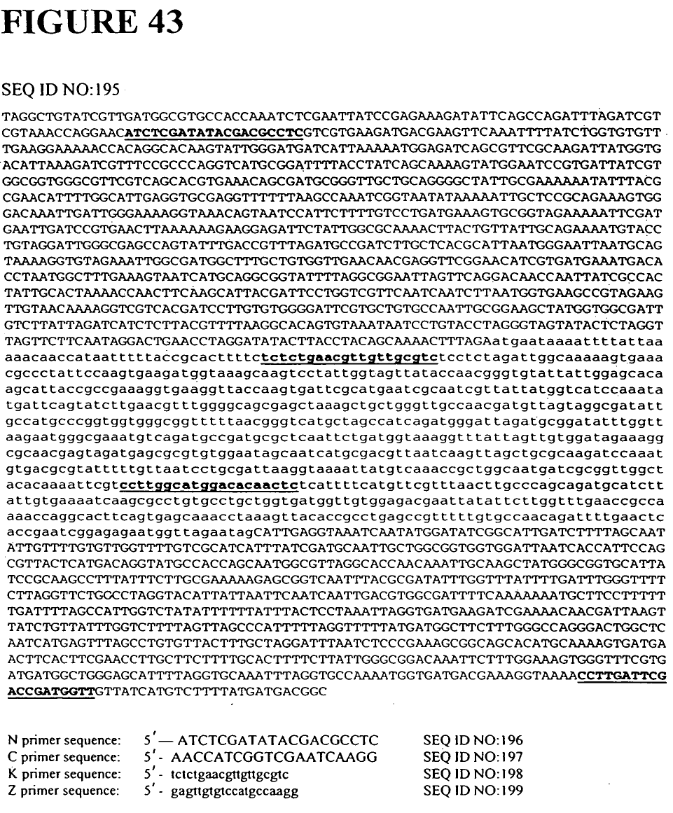

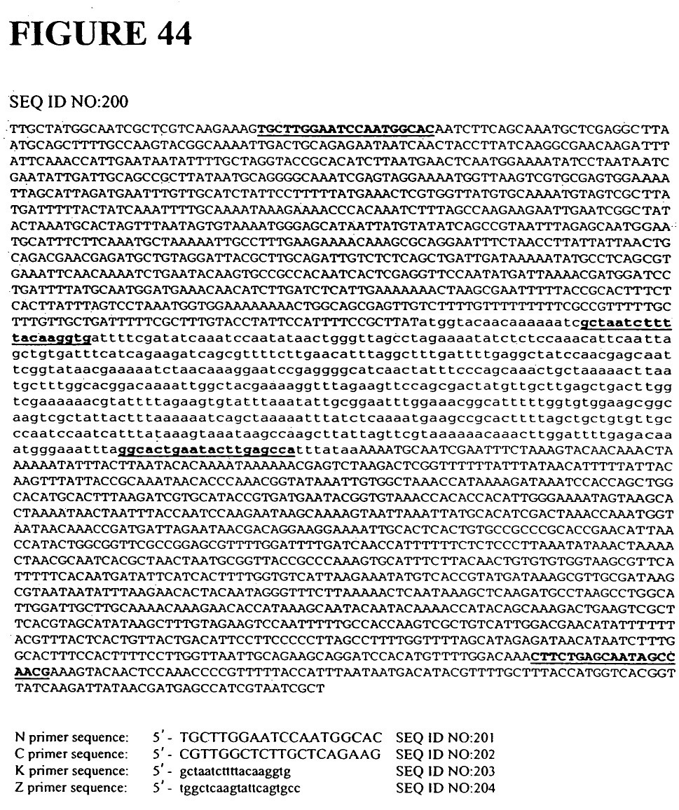

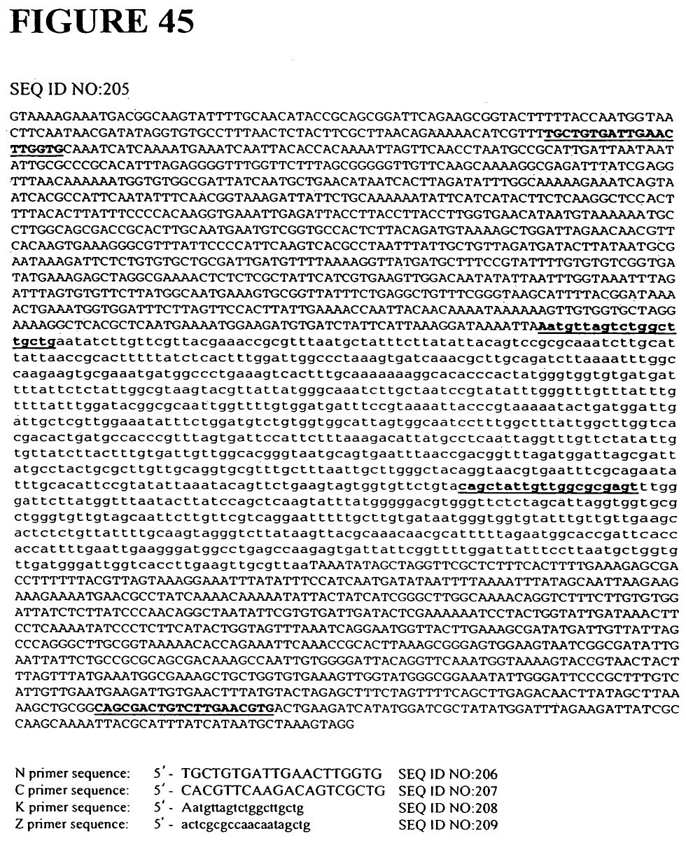

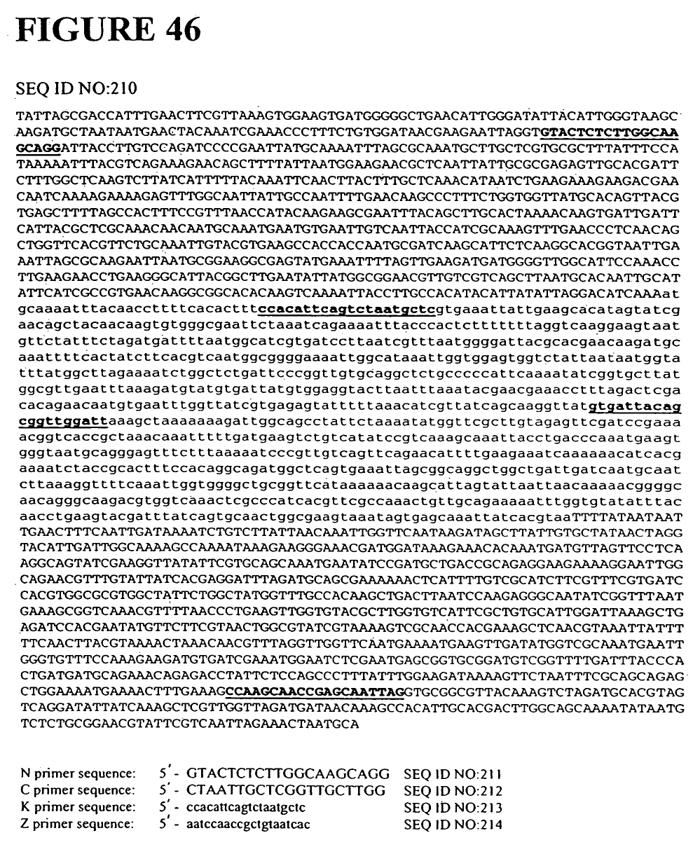

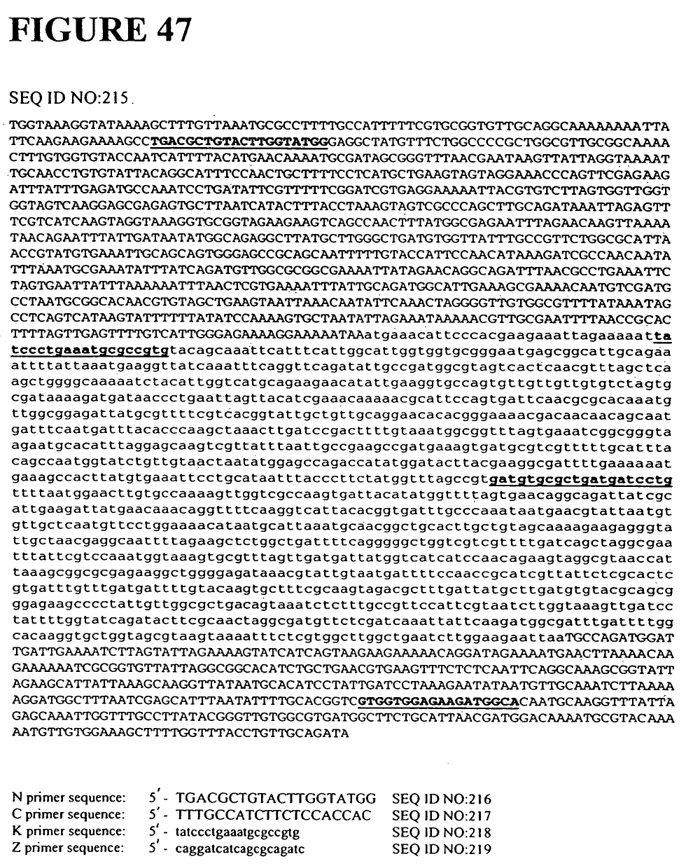

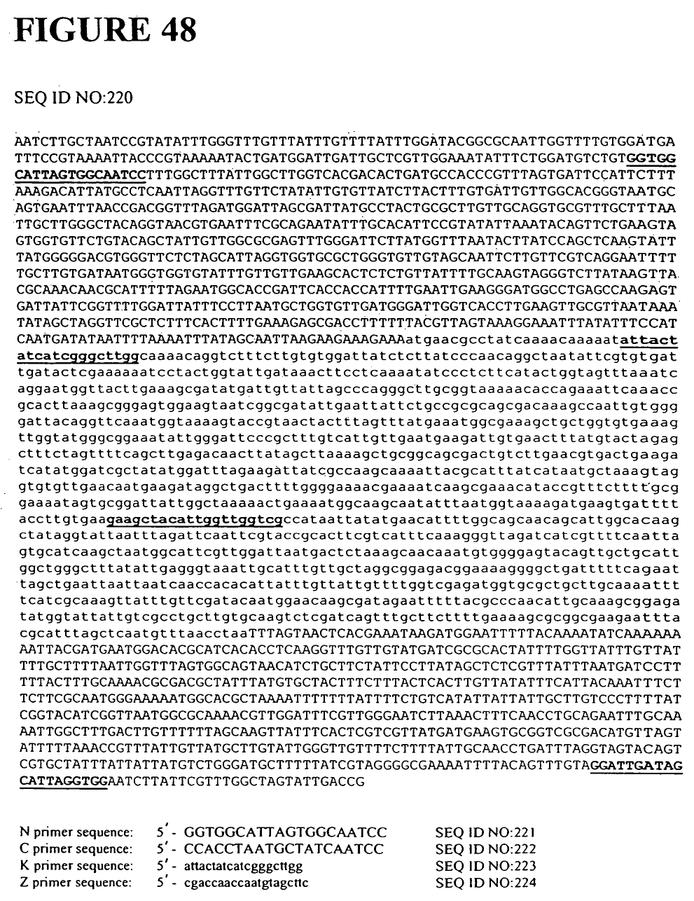

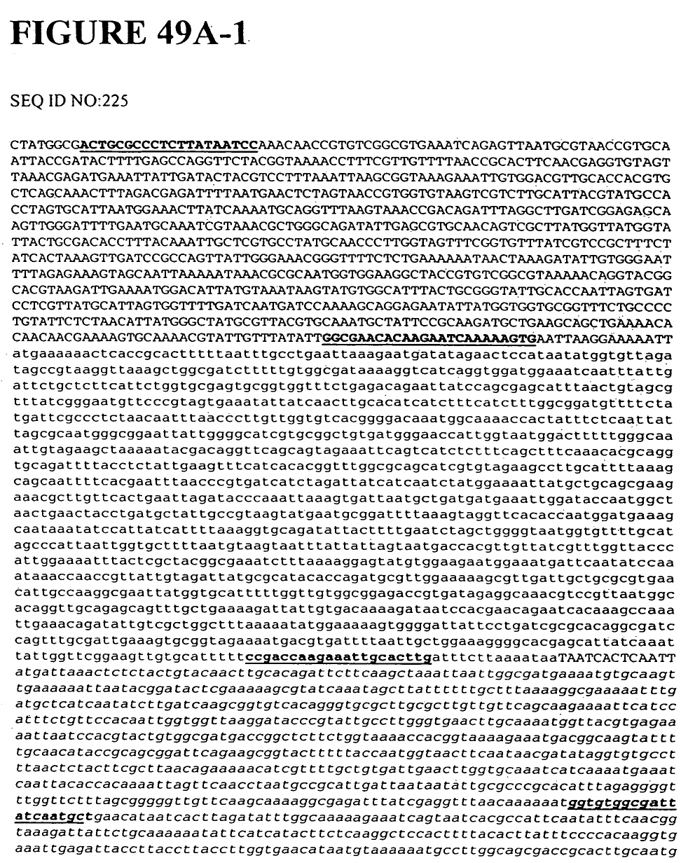

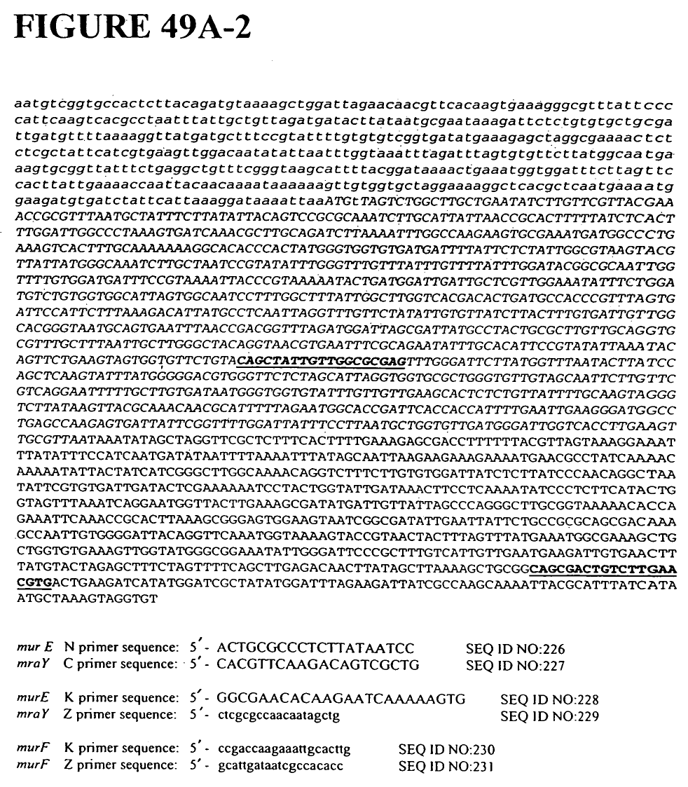

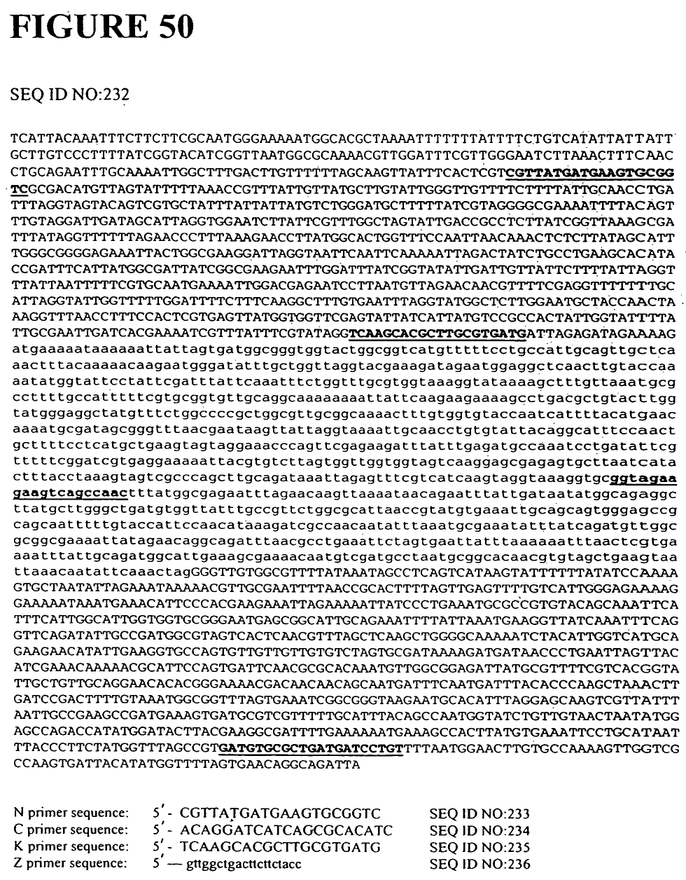

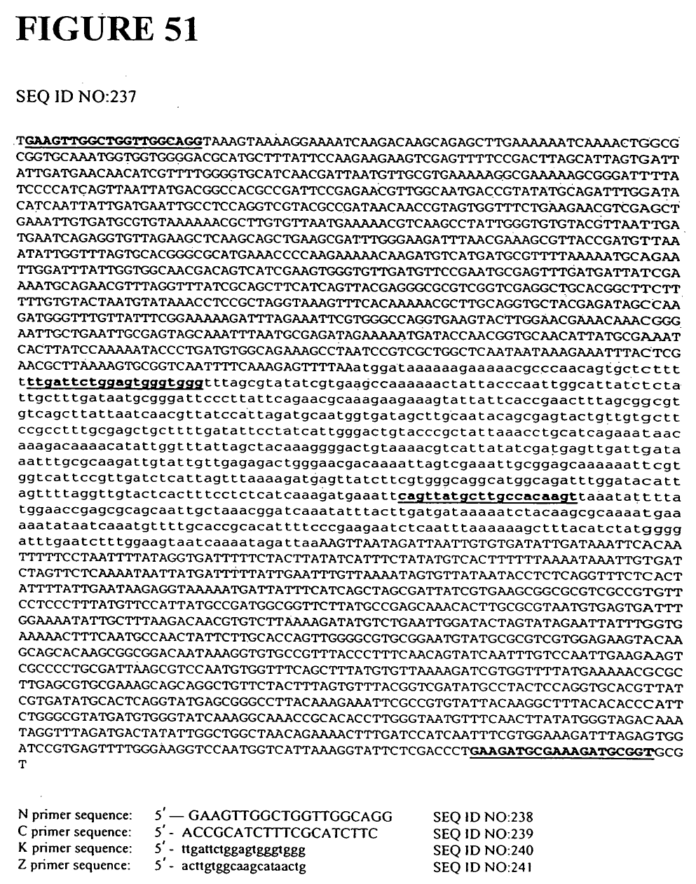

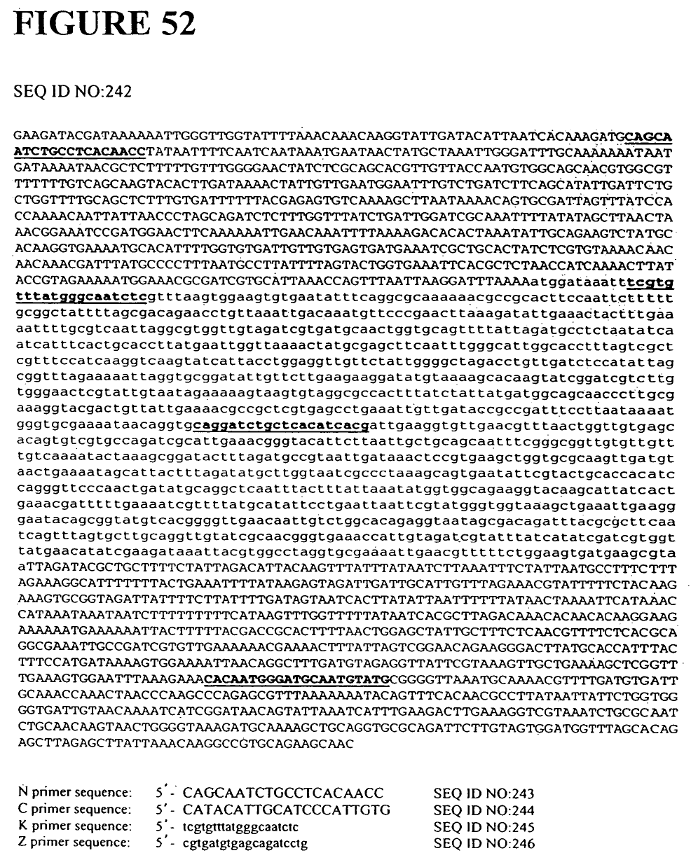

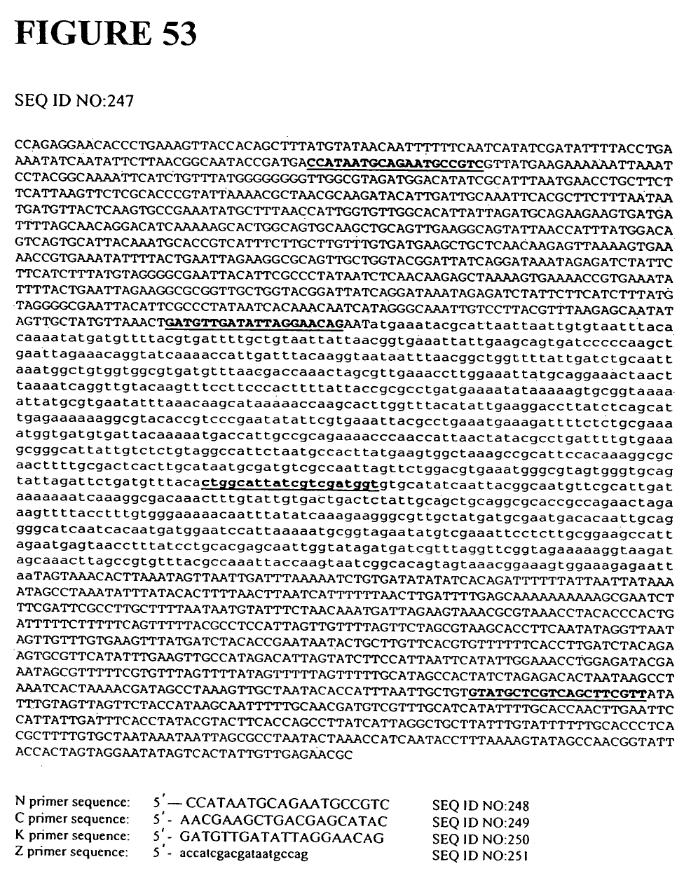

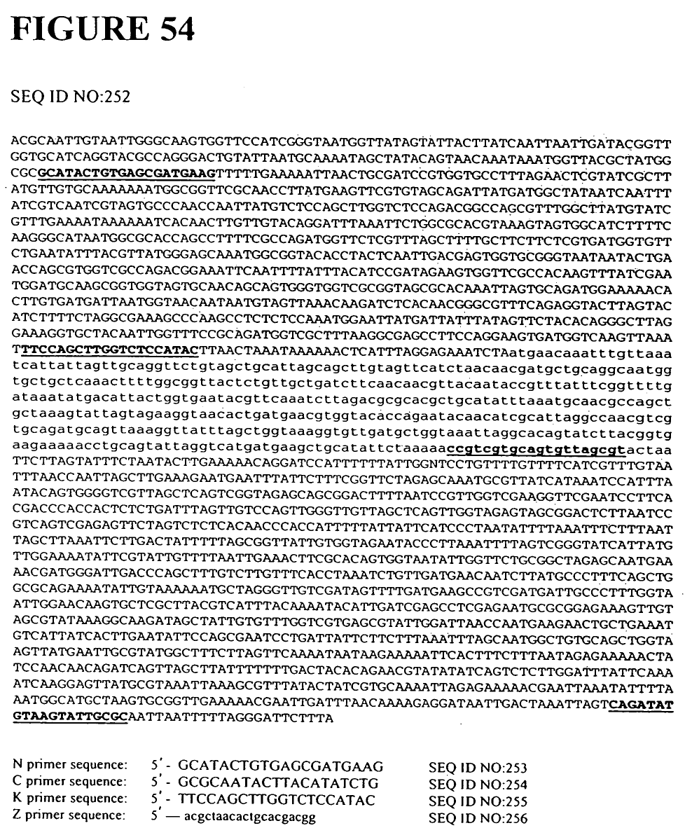

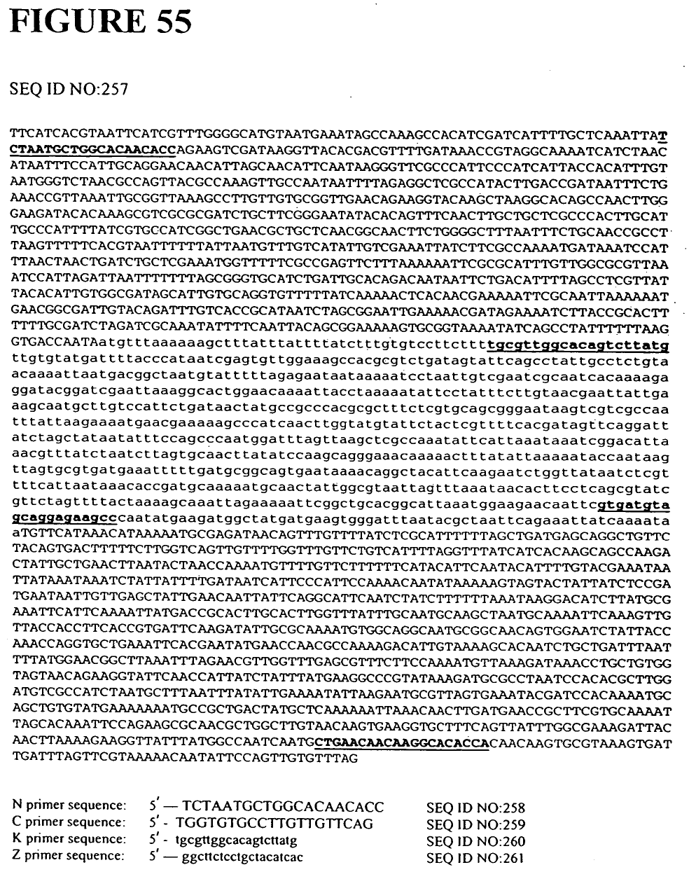

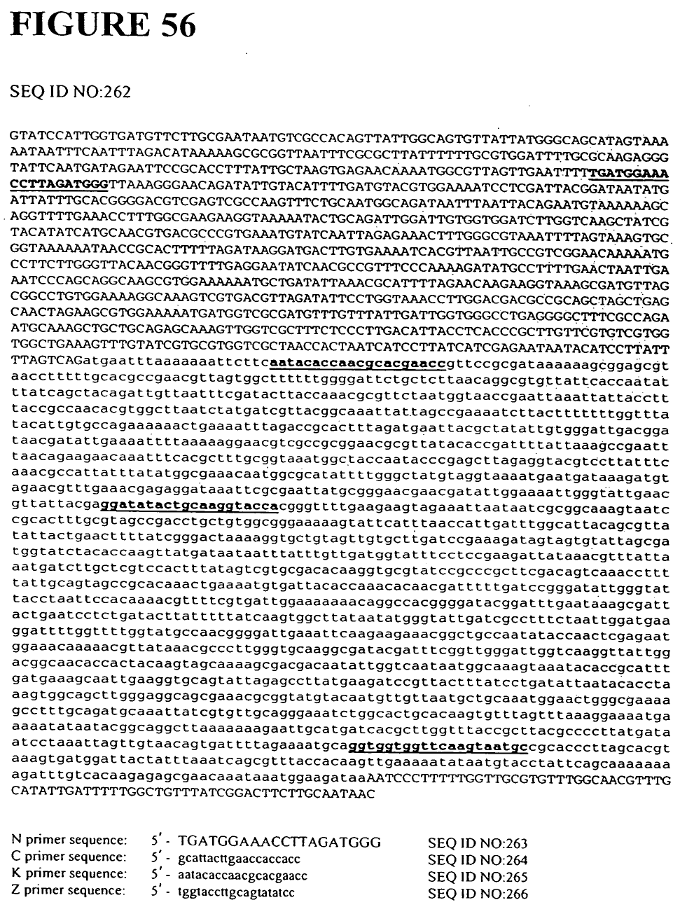

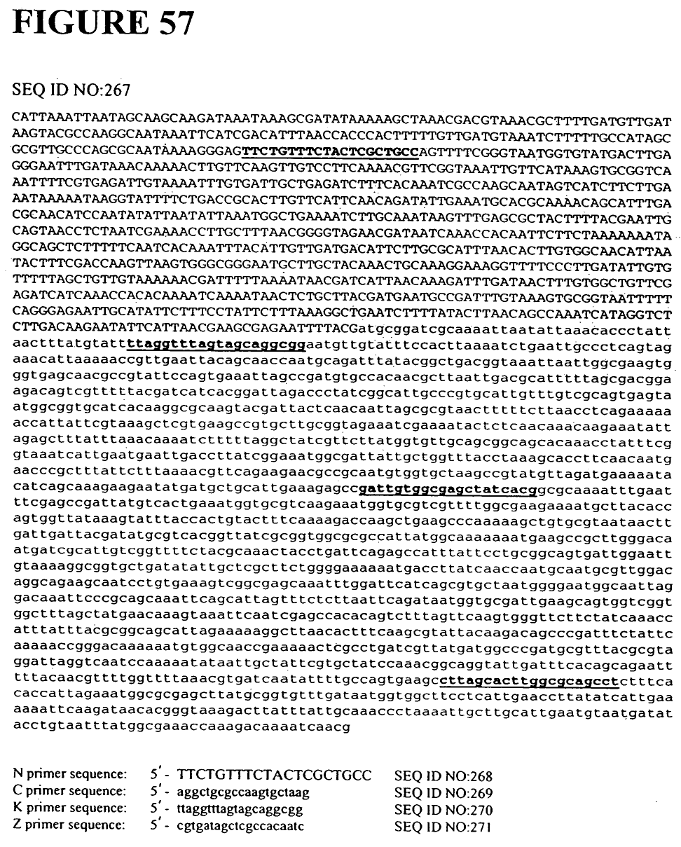

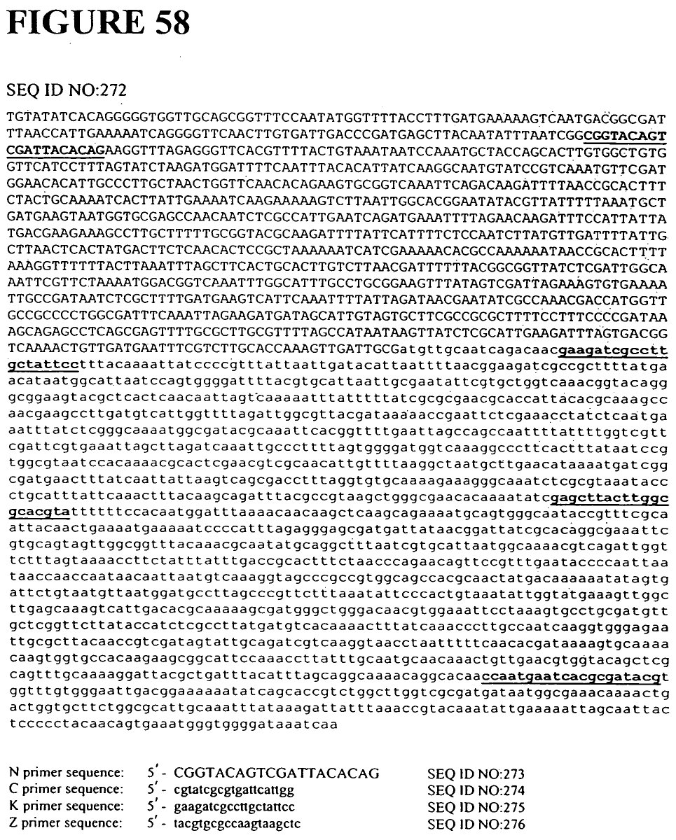

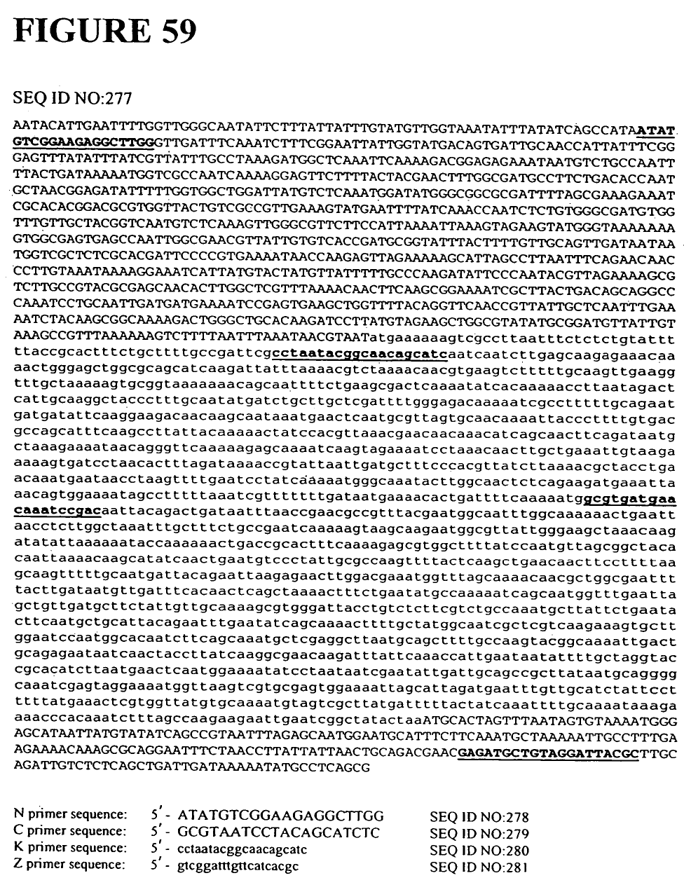

All of the gene sequences, flanking regions and PCR primer pairs are shown in FIGS. 33-59. For each gene, the PCR product was synthesized, mutagenized with the EZ::Tn <Kan-2> transposon, and transformed directly into H. influenzae. Kanamycin selected recombinants were then screened with their essential region K/Z primer pair. The results are summarized in Tables 3 and 4.

TABLE 3
The fifteen cell wall biosynthetic genes determined to be essential.

| | SEQ ID | | SEQ ID NO: of | | | No. of |
|---|---|---|---|---|---|---|
| Gene | NO:¹ | Locus² | polypeptide | Putative identification² | Essential | inserts |
| alr | 145 | HI1575 | 307 | alanine racemase | Yes | 0/96 |
| ddlB | 165 | HI1140 | 311 | D-alanine-D-alanine ligase | Yes | 0/96 |
| ftsI | 170 | HI1132 | 312 | penicillin-binding protein 3 | Yes | 0/96 |
| glmU | 180 | HI0624 | 314 | UDP-N-acetylglucosamine | Yes | 0/96 |
| | | | | pyrophosphorylase | | |
| mraY | 205 | HI1135 | 321 | phospho-N-acetylmuramoyl-pentapeptide- | Yes | 0/96 |
| | | | | transferase E | | |
| murB | 210 | HI0268 | 322 | UDP-N-acetylenolpyruvoylglucosamine | Yes | 0/96 |
| | | | | reductase | | |
| murC | 215 | HI1139 | 323 | UDP-N-acetylmuramate-alanine ligase | Yes | 0/96 |
| murD | 220 | HI1136 | 324 | UDP-N-acetylmuramoylalanine-D- | Yes | 0/96 |
| | | | | glutamate ligase | | |
| murE | 225³ | HI1133 | 325 | UDP-N-acetylmuramyl-tripeptide | Yes | 0/96 |
| | | | | synthetase | | |
| murF | 225⁴ | HI1134 | 326 | UDP-MurNAc-pentapeptide synthetase | Yes | 0/96 |
| murG | 232 | HI1138 | 327 | UDP-N-acetylglucosamine-N- | Yes | 0/96 |
| | | | | acetylmuramyl-pyrophosphoryl UDP N- | | |
| | | | | acetylglucosamine transferase | | |
| murI | 237 | HI1739 | 328 | glutamate racemase | Yes | 0/96 |
| murZ | 242 | HI1081 | 329 | UDP-NAcGlu 1-carboxyvinyltransferase | Yes | 0/96 |
| nagA | 247 | HI0140 | 340 | N-acetylglucosamine-6P-deacetylase | Yes | 0/96 |
| pal | 252 | HI0381 | 341 | outer membrane protein p6 precursor | Yes | 0/96 |

¹Refers to the coding sequence present at each SEQ ID NO.
²Information obtained through the TIGR Haemophilus influenzae KW20 locus search page (www.tigr.org/tigr-scripts/CMR2/LocusNameSearch.spl?db=ghi).
³The murE coding sequence is nucleotides 936-2429 of SEQ ID NO: 225.
⁴The murF coding sequence is nucleotides 2443-3809 of SEQ ID NO: 225.

TABLE 4
Thirteen cell wall biosynthesis genes determined to be non-essential.

| | SEQ ID | | SEQ ID NO: of | | | No. of |
|---|---|---|---|---|---|---|
| Gene | NO:¹ | Locus² | polypeptide | Putative identification² | Essential | inserts |
| amiB | 150 | HI0066 | 308 | N-acetylmuramoyl-L alanine amidase | No | 3/96* |
| dacA | | HI0029 | 309 | penicillin-binding protein 5 | No | 22/48 |
| dacB | | HI1330 | 310 | penicillin-binding protein 4 | No | 3/48 |
| glmS | | HI0429 | 311 | glucosamine-fructose-6P-transferase | No | 28/48 |
| glnA | 185 | HI0865 | 315 | glutamine synthase | No | 5/48* |
| lpp | | HI1579 | 319 | lipoprotein PCP precursor | No | 22/48 |
| mepA | | HI0197 | 320 | penicillin-insensitive murein peptidase | No | 25/48 |
| mtgA | 200 | HI0831 | 318 | peptidoglycan transglycosylase | No | 5/48* |
| pbp2 | | HI0032 | 342 | penicillin-binding protein 2 | No | 6/48 |
| pbpG | | HI0364 | 316 | penicillin-binding protein 7 (putative) | No | 25/48 |
| ponA | 267 | HI0440 | 343 | penicillin-binding protein 1A | No | 36/96* |
| ponB | | HI1725 | 344 | penicillin-binding protein 1B | No | 16/48 |
| slt | | HI0829 | 317 | soluble lytic murein transglycosylase | No | 14/48 |

*exhibit a slow growth phenotype when insertion is present in the coding sequence.
¹Refers to the coding sequence present at each SEQ ID NO.
²Information obtained through the TIGR Haemophilus influenzae KW20 locus search page (www.tigr.org/tigr-scripts/CMR2/LocusNameSearch.spl?db=ghi).

Multiple insertions were found in amiB, dacA, dacB, glmS, glnA, lpp, mepA, mtgA, murG, pbp2, pbpg, ponA, ponB, and slt. Since the K primer sequence occurred upstream of the ATG start codon for dacA, dacB, glmS, lpp, mepA, mtgA, murG and pbpG, recombinants containing an insert from each gene were sequenced with a transposon primer to determine the exact location of the insert. All of the transposons inserted within the 5′ essential region except for murG; therefore dacA, dacB, glmS, lpp, mepA, mtgA, and pbpG are non-essential. Of the ten recombinants sequenced for murG, all ten contained insertions upstream of the ATG codon; therefore, murG was determined to be essential. Only one out of 96 recombinants screened for nagA contained an insertion. Sequence analysis mapped the transposon upstream of the ATG start codon, so nagA was also determined to be essential. The K/Z primer pairs for amiB, glnA, pbp2, ponA, ponB, and slt are within the 5′ coding region; therefore, these genes are non-essential. No insertions were found within alr, ddlB, ftsI, glmU, mraY, murB, murC, murD, murE, murF, murI, murZ and pal. Recombinant colonies were then′ re-screened with their N/C primer pair to identify inserts within the flanking regions. Inserts were found in,the flanking regions of all 13 genes; therefore, alr, ddlB, ftsI, glmU, mraY, murB, murC, murD, murE, murF, murI, murZ, and pal were determined to be essential.

Discussion

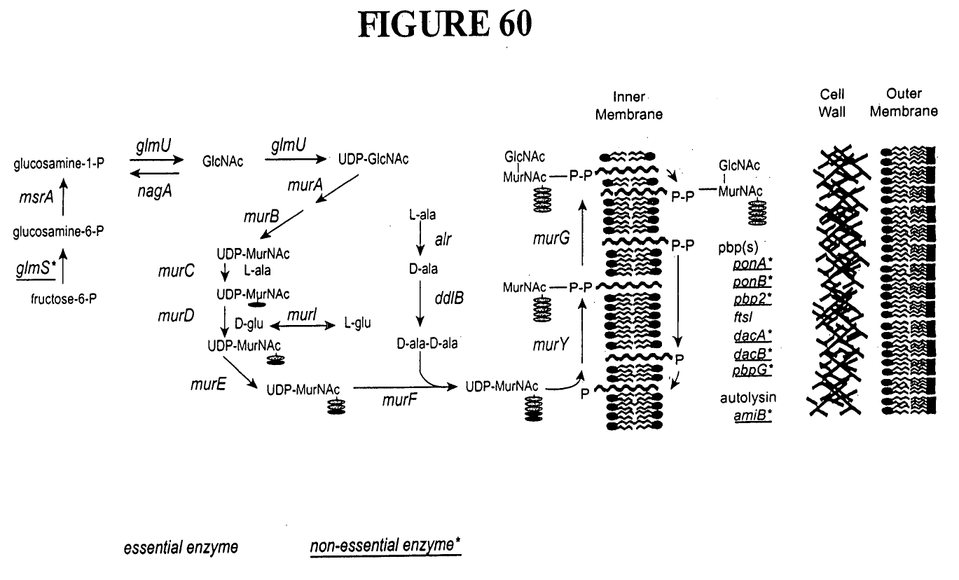

The genes necessary for the synthesis of peptidoglycan were essential (FIG. 60). The only step in the peptidoglycan biosynthesis pathway not assessed for essentiality is mrsA. The mrsA E. coli homolog (glmM) has been determined to be essential (Mengin-Lecreulx and van Heijenoort, J. Biol. Chem., 271, 32-39 (1996)). However, mrsA essentiality cannot currently be determined because H. influenzae has duplicated a segment of the chromosome that results in the presence of two identical copies of the mrsA gene. A transposon with a different antibiotic selectable marker will have to be constructed before the essentiality of mrsA can be determined.

Surprisingly, of the seven penicillin-binding proteins examined, only ftsI proved to be essential. FtsI has been shown to be essential in E. coli (Goffin et al., J. Bacteriol., 178, 5402-5409 (1996)), and the ftsI homolog in S. aureus (pbp-1) has also been shown to be essential (Wada and Watanabe, J. Bacteriol., 180, 2759-2765 (1998)).

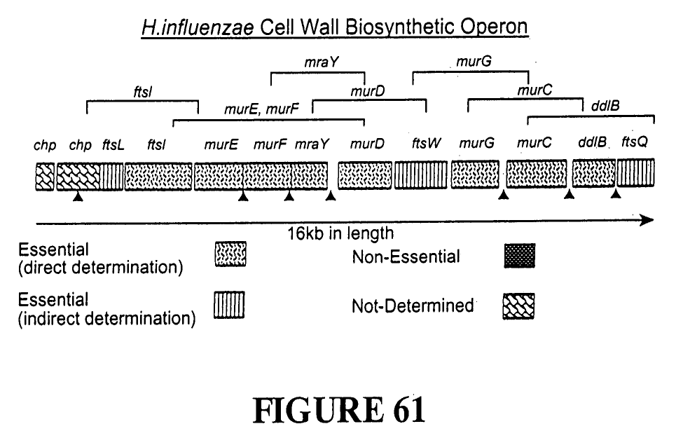

Insertional inactivation of the cell wall genes presented several new technical challenges. As shown in FIG. 61, ftsI, murE, murF, mraY, murD, murG, murC, and ddlB are tightly clustered and have the same transcriptional directionality. It is likely that all eight genes are co-transcribed as a single operon. The established protocol for determining essentiality requires screening recombinants with the K/Z primer pair, followed by re-screening with the N/C primer pair for inserts in the flanking regions if no inserts are found in the 5′ essential region. However in this operon most flanking genes are also expected to be essential, so one would not expect to find inserts within the entire mutagenized PCR fragment. Distinguishing between failed mutagenesis and true essentiality relied on detecting inserts within the small number of bases between genes. For example, the ten murG recombinants containing insertions all mapped to the 9 bases between the stop codon of ftsW and the start codon of murG. For murC, four insertions mapped to the 67 base pair intergenic region between murG and murC.

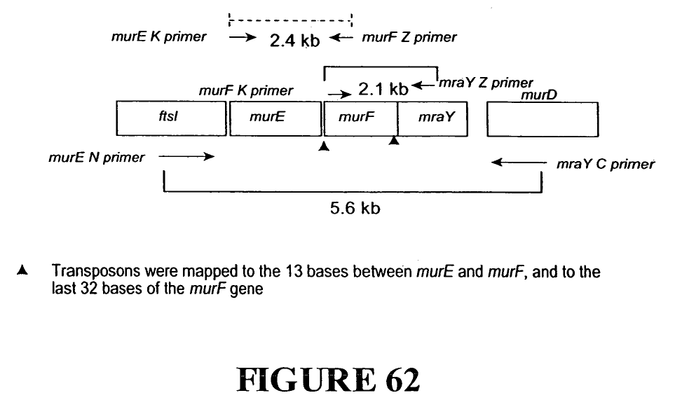

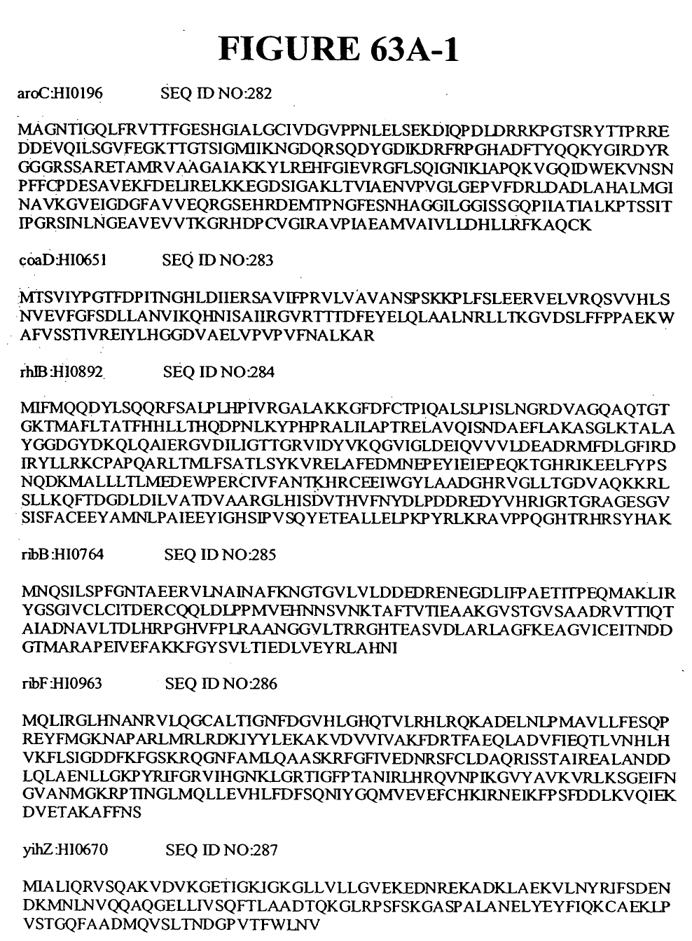

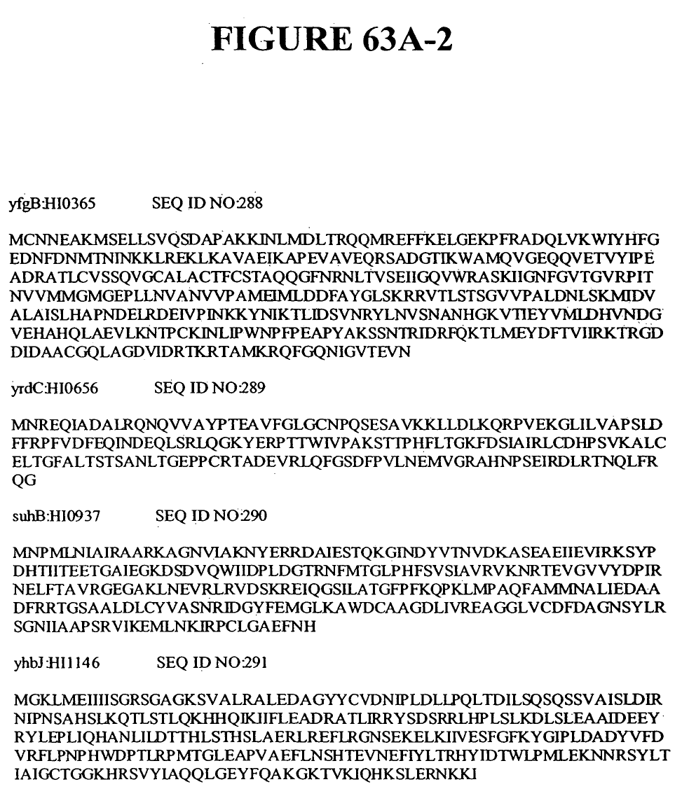

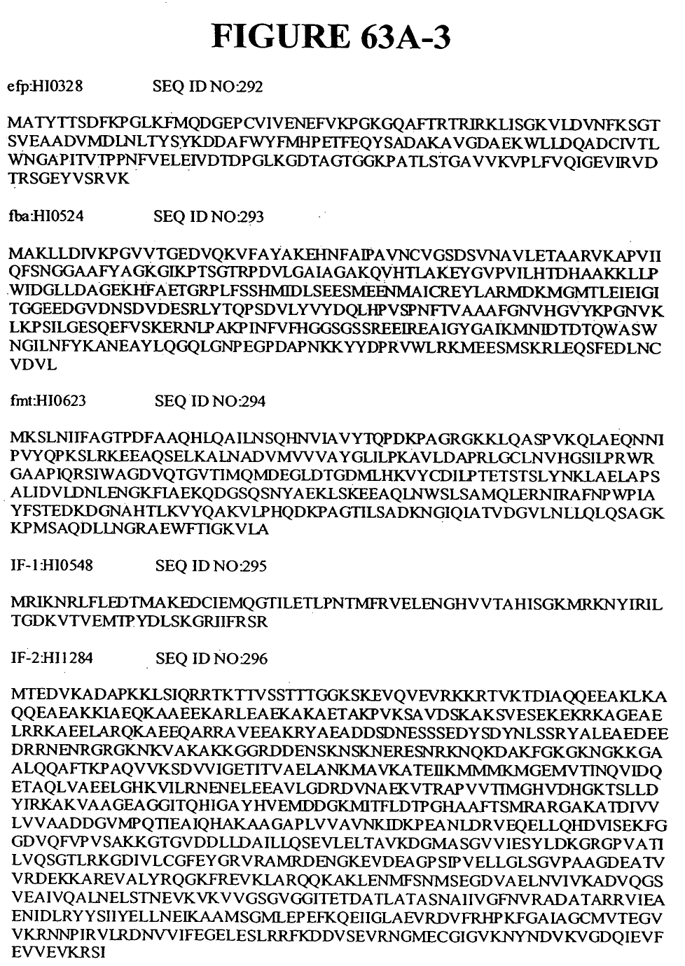

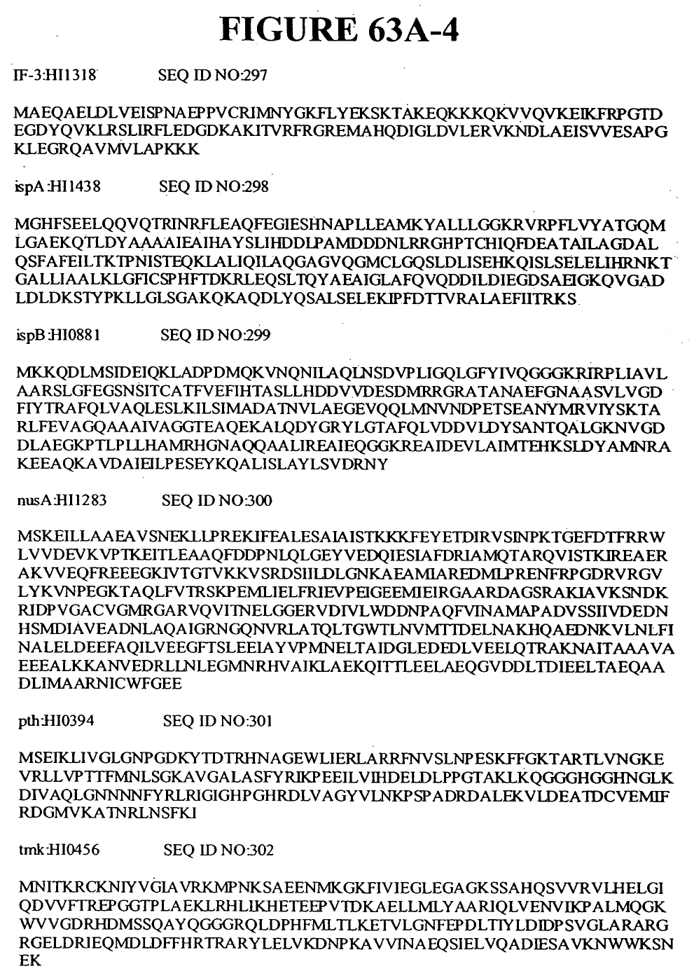

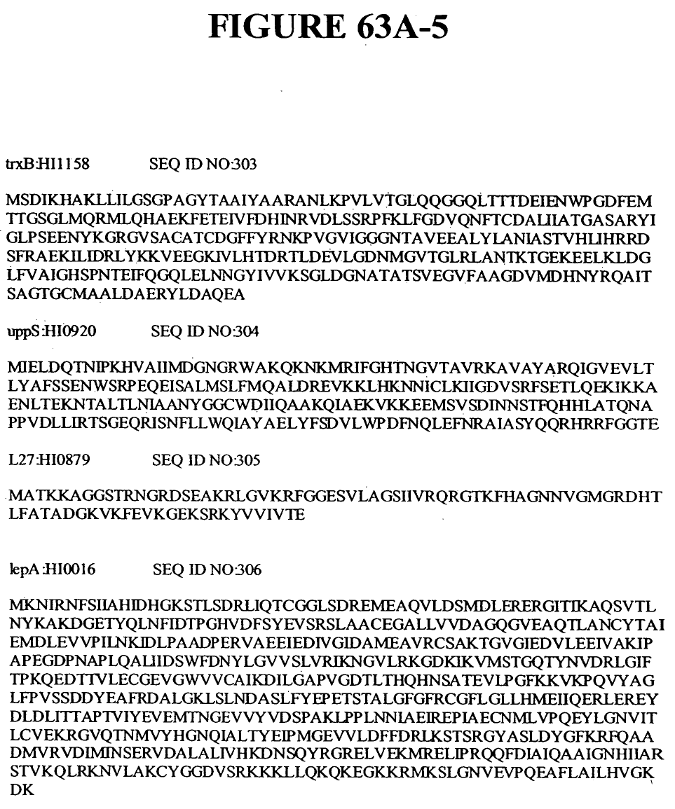

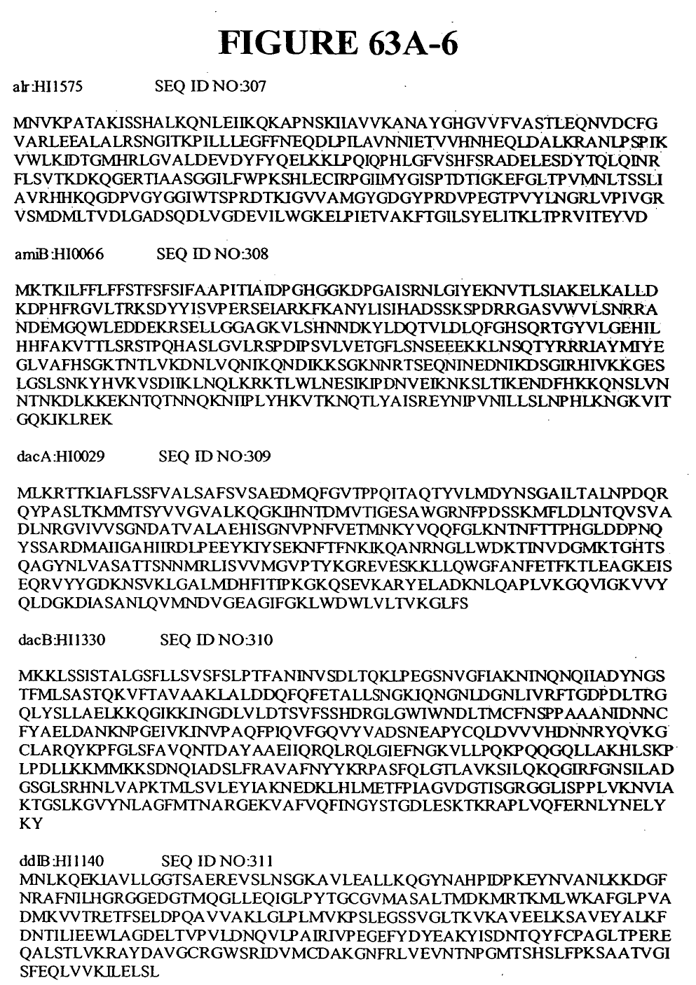

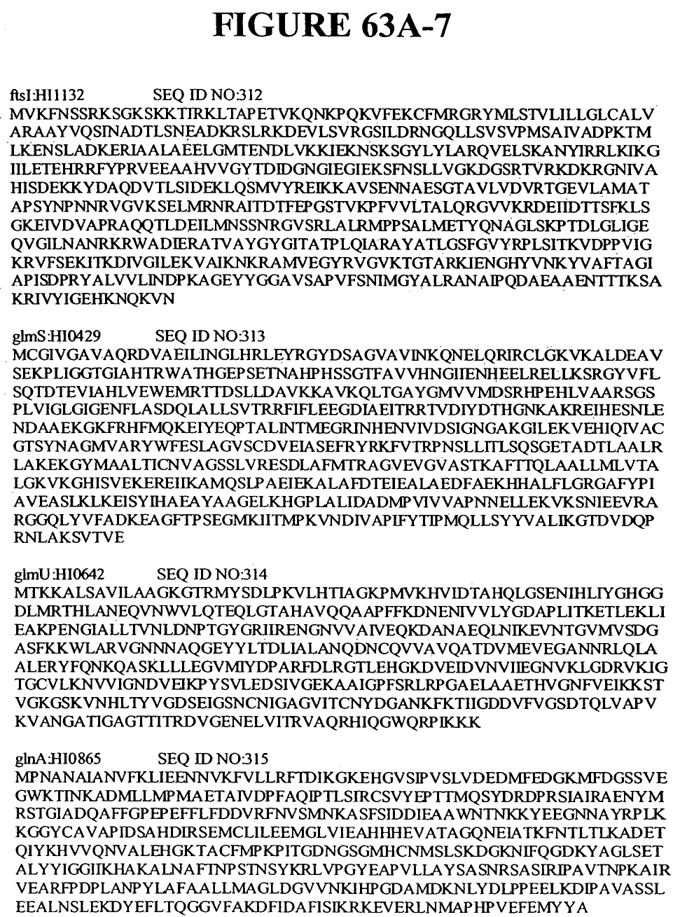

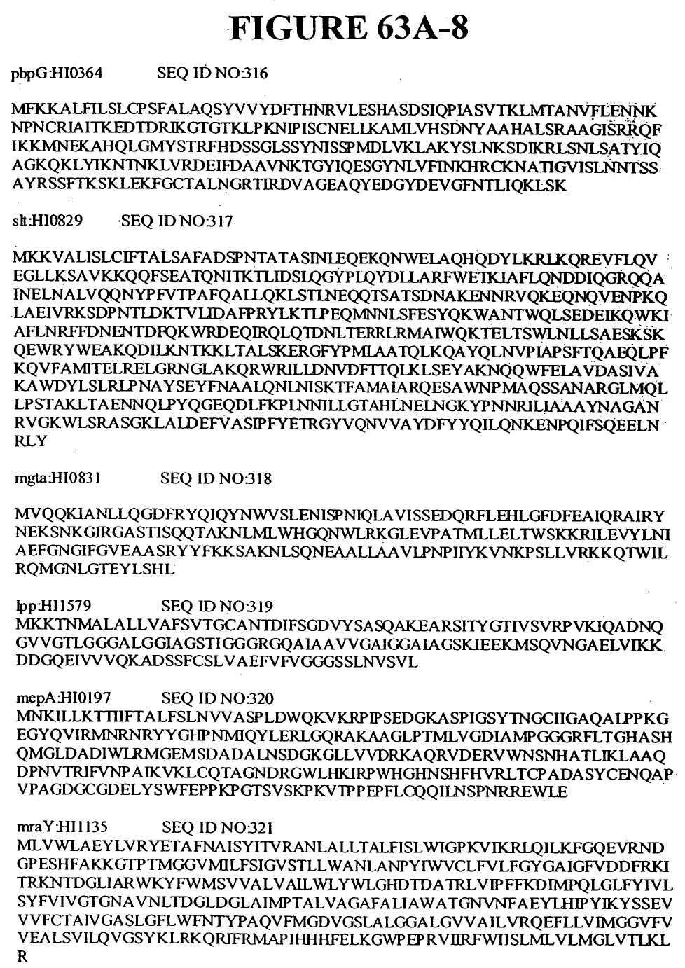

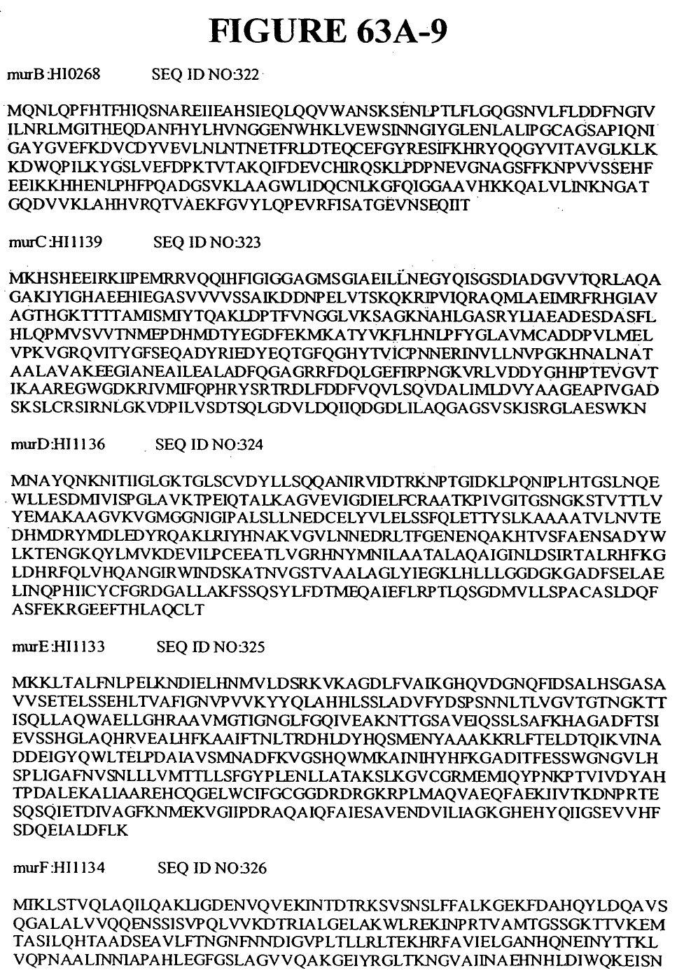

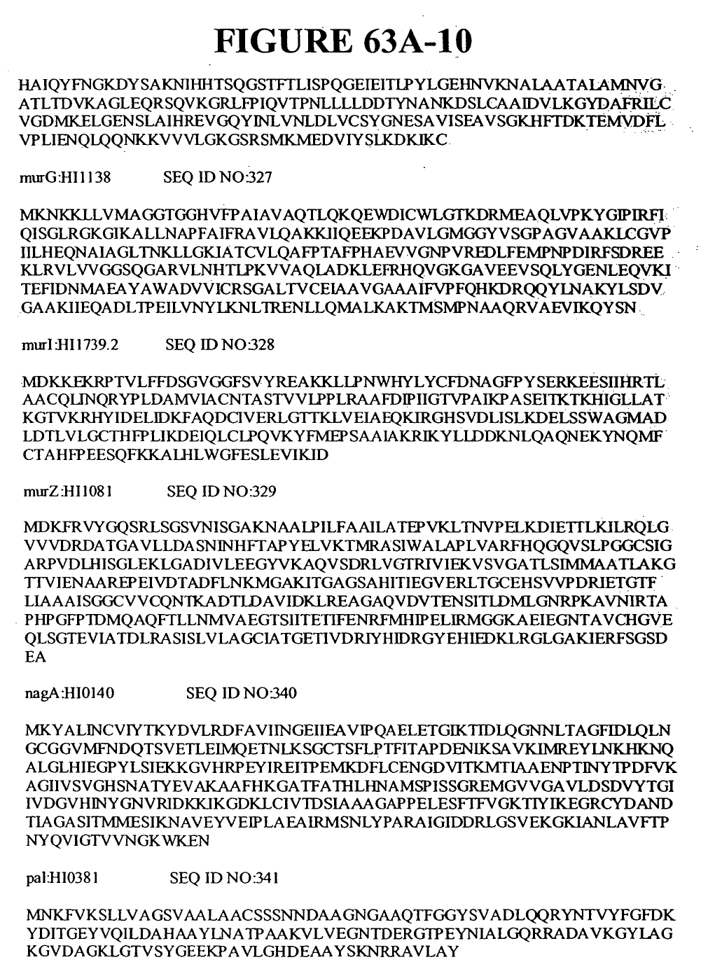

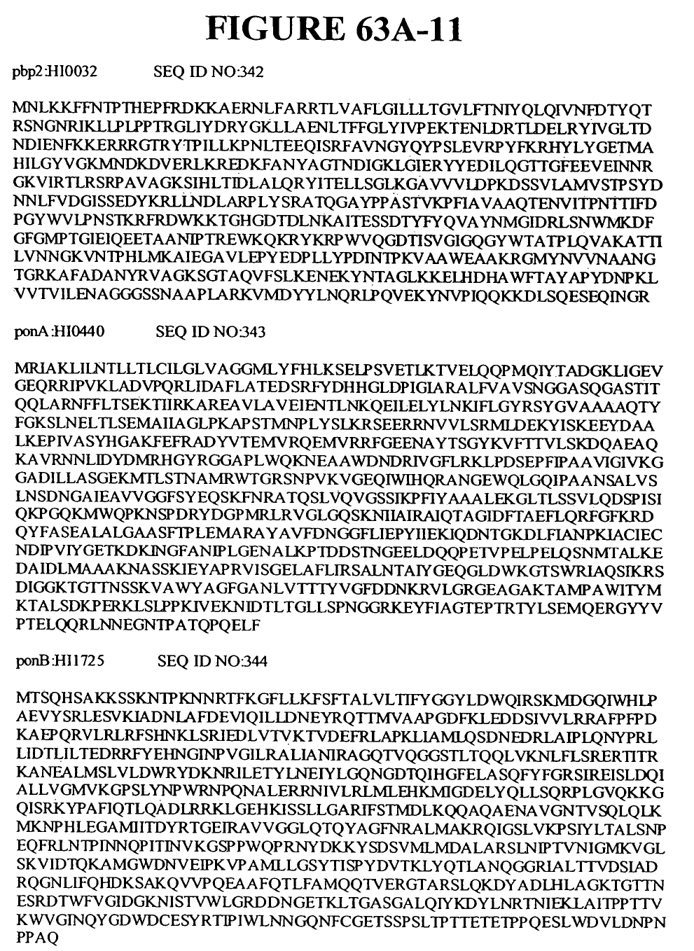

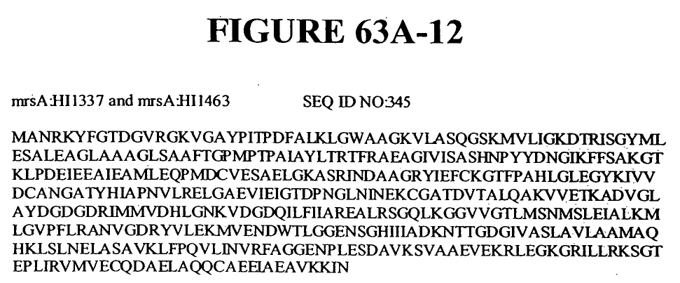

The determination of the essentiality of murE and murF was particularly challenging. In this region of the biosynthetic operon there are only 9 bases in the intergenic region between ftsI and murE, 13 bases in the intergenic region between murE and murF, and no bases between murF and mraY as the TAA stop codon of murF overlaps with the ATG start codon of mraY (FIG. 62). The largest adjacent intergenic region occurs between mraY and murD (121 bases). N/C screening of this intergenic region for mraY and murD essentiality detected numerous insertions within this 121 bases. For the mutagenesis of murE and murF, N/C primers were designed to amplify a single 5.6 kb fragment beginning upstream of murE and ending downstream of the mraY/murD intergenic region. It was anticipated that no insertions would be detected during K/Z screening of murE and murF, but that N/C screening would detect insertions within the mraY/murD intergenic region confirming that mutagenesis had occurred. However, multiple insertions were detected when recombinants were screened with either the murE-K/murF-Z primer pair or the murF-K/mraY-Z primer pair. Sequencing of the insertions revealed that the 2 unique insertions detected with the murE-K/murF-Z primer pair all mapped to the 9 base intergenic region between murE and murF. The 4 unique insertions detected with the murF-K/mraY-Z primer pair all mapped to the last 32 bases of the murF gene. Therefore, murE and murF were determined to be essential. The mapping results with murF also provide additional evidence that insertions can occur in the C-terminal end of an essential gene.

One major concern with insertional inactivation is the possibility of polar effects on downstream genes contained within an operon. Our results with the cell wall biosynthetic operon provide numerous examples of the lack of polarity of the transposon insert. Colonies containing insertions in the conserved hypothetical protein upstream of ftsI, as well as in the intergenic regions between ftsI and murE, mraY and murD, murG and murC, and murC and ddlB were all viable. Mapping studies showed that in all cases the transposon had inserted with its kanamycin gene in the same transcriptional directional as the biosynthetic genes, suggesting that the kanamycin promoter was transcribing the downstream essential genes. Both the small size of the mini-transposon and the strength of the kanamycin promoter may help to compensate for polar effects.

### Example 4

### Rapid Transposon Insertional Inactivation for Determining Essentiality of Additional H. influenzae Coding Sequences Associated with Cell Wall Biosynthesis

The H. influenzae genome contains several regions of chromosomal duplication, and one such region encompasses mrsA (glmM in E. coli). MrsA is involved in the second step of the pathway, converting glucosamine-6-phosphate to glucosamine-1-phosphate, and is encoded by both HI1337 and HI1463. In order to determine the essentiality of mrsA, two strains were constructed containing a deletion of either the HI1337 or the HI1463 mrsA gene. Each deletion strain was then independently evaluated for the insertional inactivation of the remaining mrsA gene. In both strains, the second mrsA gene could not be insertionally inactivated; therefore mrsA is essential. A related cell wall biosynthesis study was undertaken to determine if the essential gene murI (glutamate racemase) could be insertionally inactivated in the presence of D-glutamate. MurI is essential in E. coli, but murI mutants auxotrophic for D-glutamate have been identified (Doublet et al., J. Bacteriol., 175, 2970-2979 (1993)). We were unable to generate H. influenzae murI mutants in the presence of D-glutamate suggesting that H. influenzae, unlike E. coli, does not transport D-glutamate.

Materials and Methods

Bacterial strains and growth conditions: H. influenzae Rd was obtained from the American Type Culture Collection as ATCC 51907 and grown in brain-heart infusion supplemented with 5% Fildes Enrichment (sBHI) at 37° C. in 5% CO₂. Plasmid pACYC 184 was purchased from New England Biolabs. D-glutamate (Sigma G-1001) was supplemented at 100 mg/ml. sBHI selection plates were supplemented with 1 mg/ml chloramphenicol (CM), 30 mg/ml kanamycin (KAN) or a combination of both CM and KAN.

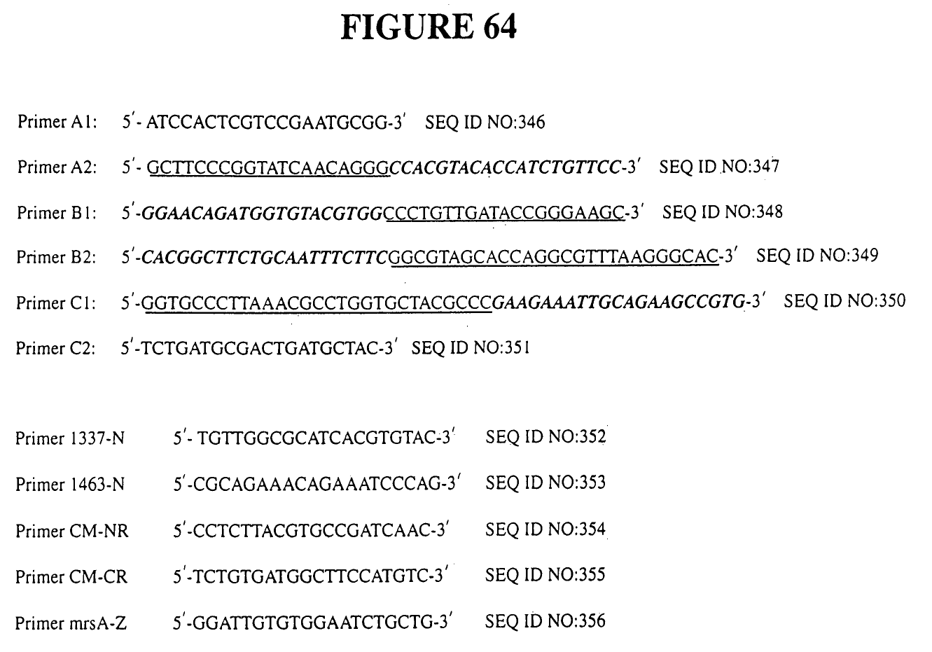

Oligonucleotide synthesis: All of the oligonucleotides used in this report were synthesized by Genosys and are shown in FIG. 64.

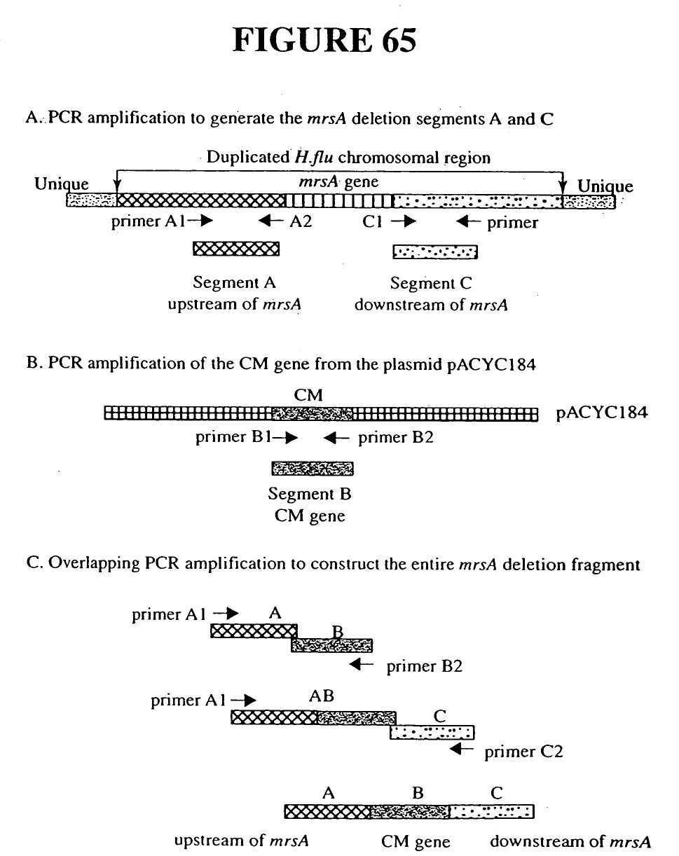

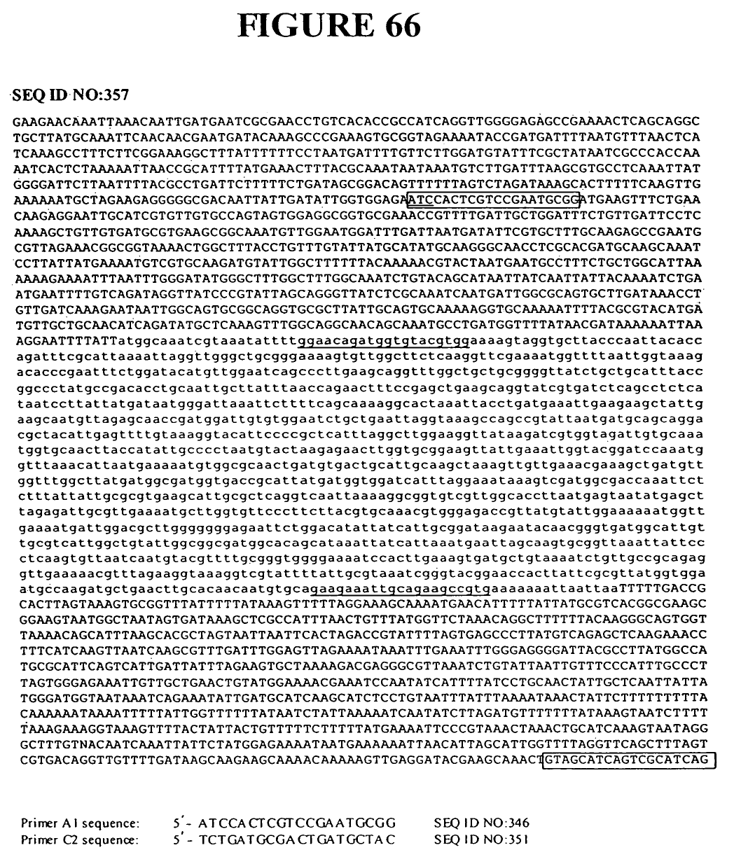

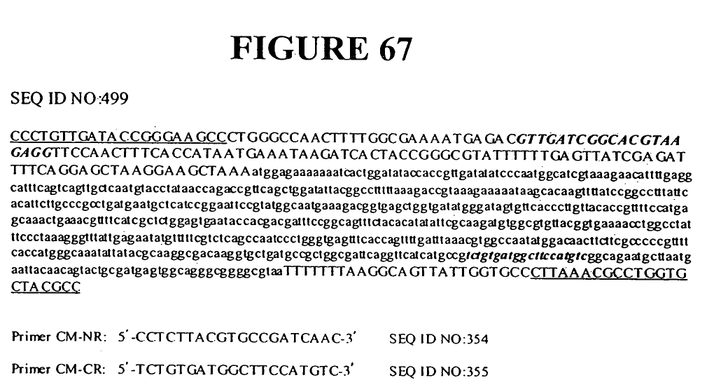

Construction of the mrsA gene deletion fragment: The PCR amplification scheme used to construct the mrsA gene deletion fragment is outlined in FIG. 65. The deletion fragment, containing a replacement of the mrsA gene (FIG. 66) with the selectable marker for chloramphenicol resistance, was first synthesized in three segments. Segment A contains sequence upstream of mrsA amplified from primers A1 (500 bp upstream of mrsA) and primer A2 (an overlapping primer containing the 5′ end of mrsA fused to the 5′ end of the CM gene). Segment B amplifies the CM gene (FIG. 67) with primers B1 and B2 (containing overlapping extensions fused to the 5′ and 3′ ends of mrsA). Segment C contains sequence downstream of mrsA amplified from primers C1 (an overlapping primer containing the 3′ end of the CM gene fused to the 3′ end of mrsA) and primer C2 (1000 bp downstream of mrsA). Segments A and C were amplified from 1 mg of H. influenzae genomic DNA whereas the CM gene was amplified from the 10 ng of E. coli plasmid pACYC184 using Gibco BRL Platinum Taq polymerase as follows: 94° C. for 5 min (1 cycle);. 94° C. for 30 sec, 55° C. for 1 min and 72° C. for 1.5 mm (25 cycles); and 72° C. for 5 min. After amplification and gel purification, segments A and B were re-amplified into a single AB fragment by overlapping PCR using primers A1 and B2. Another round of overlapping PCR using primers A1 and C2 was then performed combining segment AB with segment C to produce a single mrsA deletion fragment ABC.

Chromosomal DNA isolation. Chromosomal DNA was isolated from strains 1337D and 1463D following the enclosed directions of the Qiagen DNeasy Tissue Kit.

Transposon mutagenesis and PCR colony screening: The protocols for Epicentre EZ::TN <KAN-2> mutagenesis, transformation into H. influenzae, and PCR screening of recombinant colonies was performed as described herein with the following modification: competent cells of 1337D and 1463D transformed with the mutagenized mrsA fragment were plated onto sBHI plates supplemented with CM and KAN.

Results

Determination of MrsA essentiality: Construction of the deletion strains began with transforming the mrsA deletion fragment ABC into competent cells of H. influenzae and plating onto sBHI supplemented with 1 mg/ml CM. Since segments A and C were synthesized from sequence contained within the duplicated chromosomal region, the mrsA deletion fragment could recombine at either the HI1337 or HI1463 mrsA coding sequence. Surviving colonies were first screened with primers 1337-N or 1463-N (unique sequences upstream of the duplicated mrsA chromosomal region) and primer CM-NR (the 5′ end of the CM coding sequence) to determine which mrsA coding sequence had undergone recombination. Of 16 colonies screened with the 1337-N/CM-NR primer set, 12 appeared to contain a deletion of the 1337 mrsA coding sequence. Of the 16 additional colonies screened with the 1463-N/CM-NR primer set, 5 appeared to contain a deletion of the 1463 mrsA coding sequence. Two colonies of each were purified and subjected to additional PCR analysis with primer sets 1337-C/CM-CR and 1463-C/CM-CR to confirm the presence of the 3′ end of the CM coding sequence, as well as primer sets 1337-N/mrsA-Z and 1463-N/mrsA-Z to confirm the absence of the respective mrsA coding sequence. One strain of each was selected and designated 1337D and 1463D.

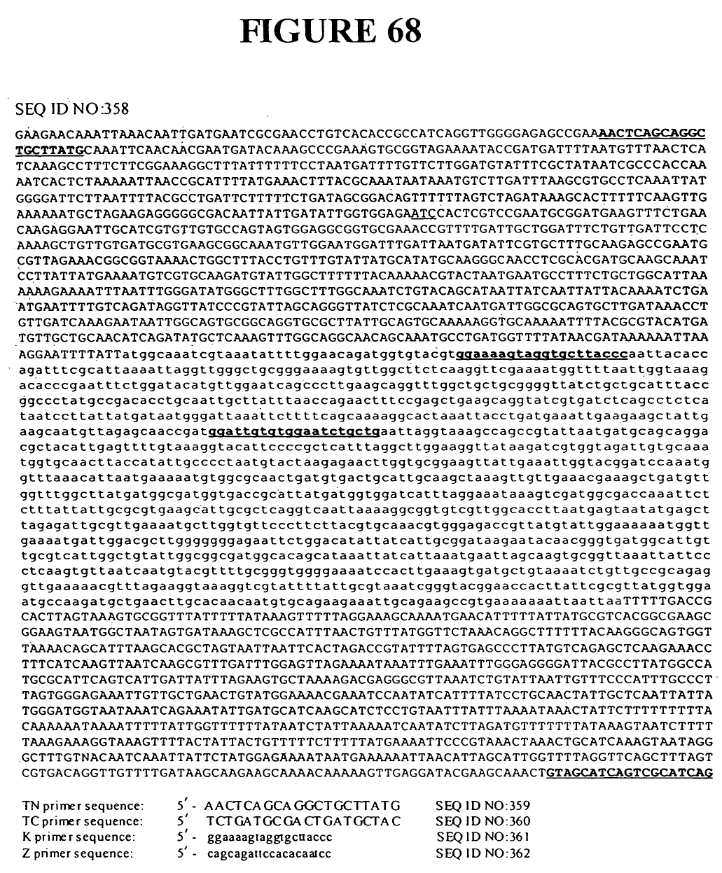

The mrsA coding sequence, the flanking regions, and the PCR primer pairs are shown in FIG. 68. The PCR product was synthesized, mutagenized with the EZ::Tn <KAN-2> transposon, and transformed directly into competent cells of either 1337D or 1463D. To maintain the CM replacement of the deleted mrsA coding sequence and to select for the recombination of the remaining mrsA coding sequence, the transformants were plated onto sBHI plates supplemented with 1 mg/ml CM and 30 mg/ml KAN. Ninety-six colonies from each transformation were then screened with the mrsA essential region K/Z primer pair. No insertions in the remaining mrsA coding sequence were found in either 1337D or 1463D. Insertions were found, however, within the flanking regions; therefore, mrsA was determined to be essential.

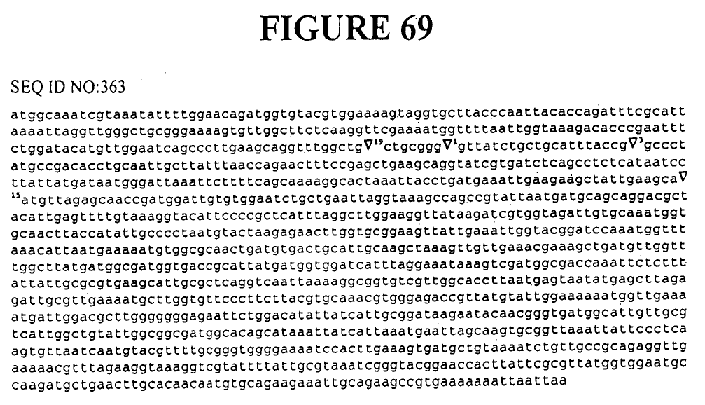

To further demonstrate the ability of the mutagenized mrsA PCR product to recombine into the chromosome, the PCR product was transformed into wild type H. influenzae. Kanamycin resistant colonies were then screened for the presence of Tn insertions within one of the two mrsA coding sequences. Of the eight colonies screened with the mrsA KZ primer pair, four were found to contain two PCR products. The 439 bp band represented the KZ fragment with no Tn insert and the 1660 bp band represented the KZ fragment containing the 1221 bp transposon, indicating that within these four colonies one of the two mrsA coding sequences was insertionally inactivated. Additional PCR analysis showed that two of the colonies contained insertions in HI1337 and the remaining two colonies contained insertions in HI1463. All four colonies (1337::Tn#1, 1337::Tn#3, 1463::Tn#15 and 1463::Tn#19) were subsequently sequenced to confirm the insertion of the transposon within the identified mrsA-terminus (FIG. 69).

The N/C primer pair was then used on chromosomal DNA isolated from strains 1337::Tn#1 and 1463::Tn#19 to generate PCR products containing Tn insertions within each mrsA coding sequence. The PCR product from 1337::Tn#1 was used to transform 1463D, and the PCR product from 1463::Tn#19 was used to transform 1337D in an attempt to replace the remaining mrsA coding sequence with an insertionally inactivated mrsA coding sequence. Transformed cells were plated onto sBHI supplemented with 1 mg/ml CM and 30 mg/ml KAN. Of the ninety-six colonies screened from each transformation, none contained insertions within′ the remaining mrsA coding sequence. To confirm that the insertionally inactivated mrsA coding sequences could recombine into the chromosome, the 1337::Tn#1 and 1463::Tn#19 PCR products were individually transformed into wild type H. influenzae. Kanamycin resistant colonies were then screened for the presence of Tn insertions within the respective mrsA coding sequence. Of the 16 colonies screened from each transformation, all 16 contained insertions in the respective mrsA coding sequence. This provided final confirmation that phosphoglucosamine mutase is an essential enzymatic step in H. influenzae cell wall biosynthesis.

Insertional inactivation of murI in the presence of D-glutamate. In order to determine if insertionally inactivated murI mutants auxotrophic for D-glutamate could be obtained in H. influenzae, wild type H. influenzae was transformed with the Tn mutagenized murI PCR fragment and plated onto sBHI supplemented with mg/ml KAN and 100 mg/ml D-glutamate. Of the ninety-six colonies screened, none contained an insertion in murI. Therefore, H. influenzae murI mutants cannot be obtained by supplementing with D-glutamate. This finding suggests that H. influenzae is unable to take up D-glutamate.

### Example 5

### Identification of Essential and Non-Essential Ribosomal Genes in Haemophilus influenzae

Many antimicrobial compounds target the bacterial ribosome. This list includes the oxazolidinones, the macrolides, the aminoglycosides, the streptogramins, the tetracyclines, lincomycin, and chloramphenicol. Recently the 50S and 30S ribosomal subunits have been crystallized (Ramakrishnan et al., Cell, 108(4), 557-72 (2002)), thus making it possible to use structural design in the development of new anti-ribosomal compounds as well as improving known anti-ribosomal classes. To determine which ribosomal genes are essential in Haemophilus influenzae, the essentiality of all 58 H. influenzae ribosomal genes was systematically determined by targeted gene disruption. With this method, a PCR amplicon consisting of the target gene and flanking sequences is mutagenized in vitro using the EZ::TN <Kan-2> transposon and transformed directly into H. influenzae. Recombinant colonies are then screened by PCR for the presence of a transposon insert within the 5′ essential region of the target gene. If an insert is found, the gene is determined to be non-essential. If no inserts are found within the 5′ essential region but are found within the flanking sequences, then the gene is determined to be essential.

Materials and Methods

Bacterial strains and growth conditions: Haemophilus influenzae Rd was obtained from the American Type Culture Collection as ATCC 51907 and grown in brain-heart infusion supplemented with 5% Fildes Enrichment (sBHI) at 37° C. in 5% CO₂. Transformants were plated on sBHI supplemented with either 30 mg/ml kanamycin (Kan), 1 mg/ml chloramphenicol (Cm), or both (Kan/Cm).

Oligonucleotide synthesis: All of the oligonucleotides used in this report were synthesized by Genosys.

Tn mutagenesis and PCR colony screening: The protocols for Epicentre EZ::Tn <Kan-2> mutagenesis, transformation into H. influenzae, PCR screening of recombinant colonies, and sequence determination of the Tn insertion site are as described in the previous examples.

Gene deletion protocol: The protocol for deleting H. influenzae genes by replacement with an antibiotic resistance marker is as described in the previous examples.

PCR sequencing: PCR products synthesized with either the K/Z or N/C primer pairs were isolated using the Qiagen MinElute columns. One hundred nanograms of PCR product, 100 ng of Epicentre Kan-2 FP-1 transposon sequencing primer and sterile water to a final volume of 12 ml were added to a single tube and subjected to rough draft sequencing.

Results and Discussion

There are a total of 58 ribosomal genes in H. influenzae. The 30S ribosomal genes include rpsA, rpsB, rpsC, rpsD, rpsE, rpsF, rpsG, rpsH, rpsI, rpsJ, rpsK, rpsL, rpsM, rpsN, rpsO, rpsP, rpsQ, rpsR, rpsS, rpsT and rpsU. The 50S ribosomal genes include rplA, rplB, rplC, rplD, rpE, rplF, rplI, rplJ, rplK, rplL, rplM, rplN, rplO, rplP, rplQ, rplR, rplS, rplT, rplU, rplV, rplW, rplX, rplY, rpmA, rpmB, rpmC, rpmD, rpmE, rpmF, rpmG, rpmH, rpmI, and rpmJ. Three additional genes associated with ribosome assembly include rimK (probable 50S protein S6 modification protein), rimL (ribosomal protein alanine transferase) and prmA (50S ribosomal protein L11 methyltransferase).

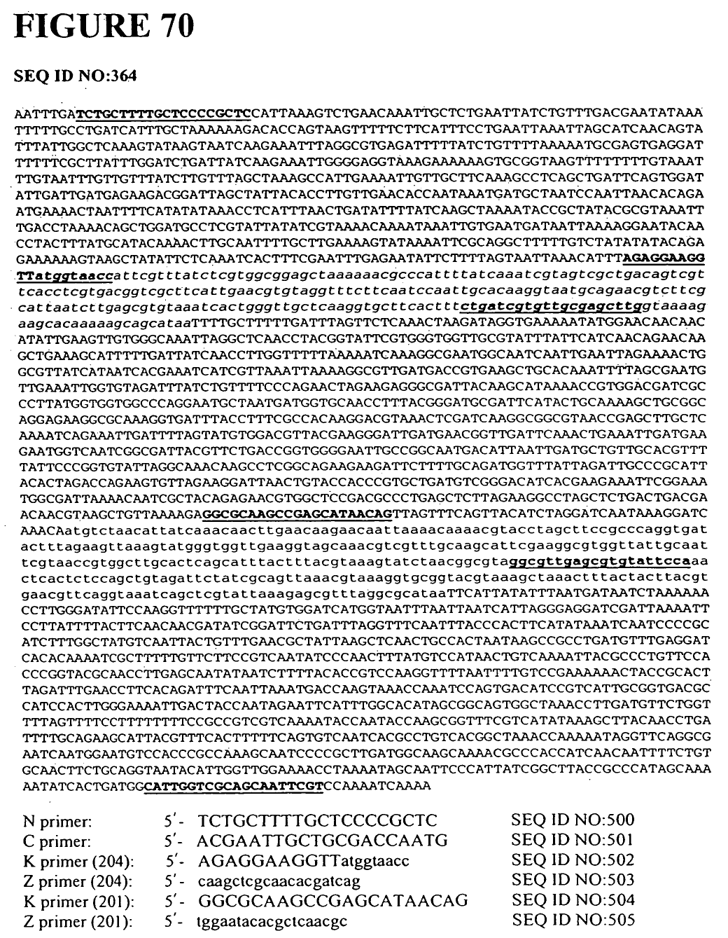

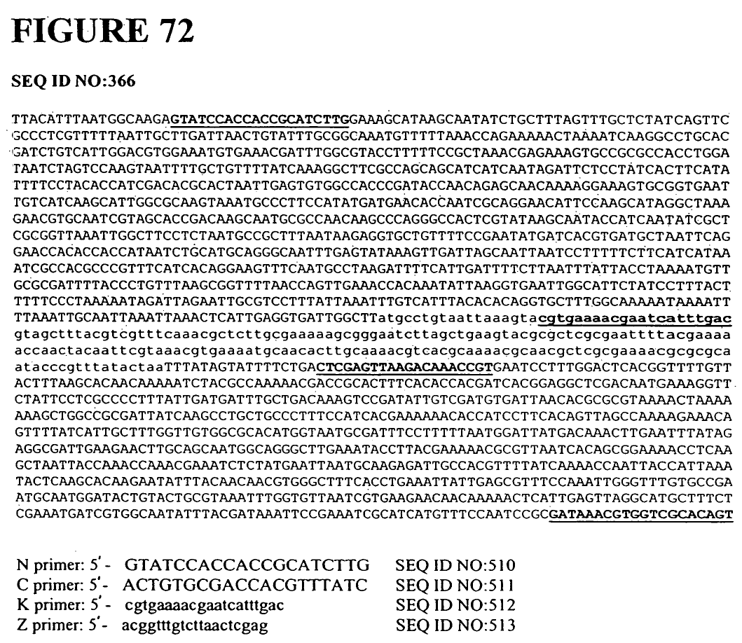

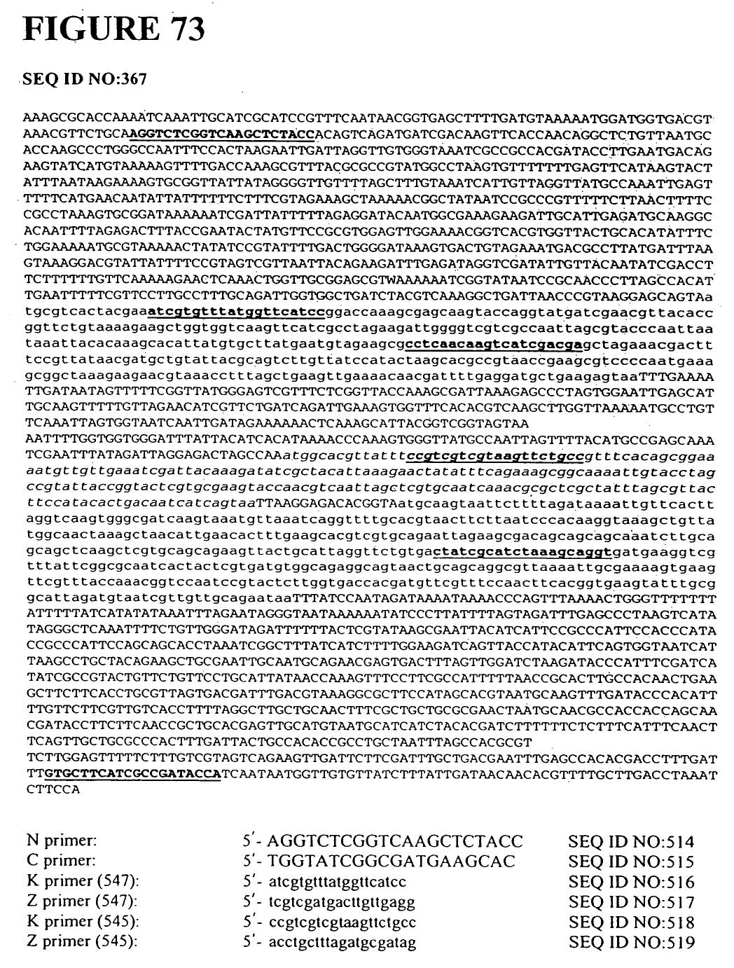

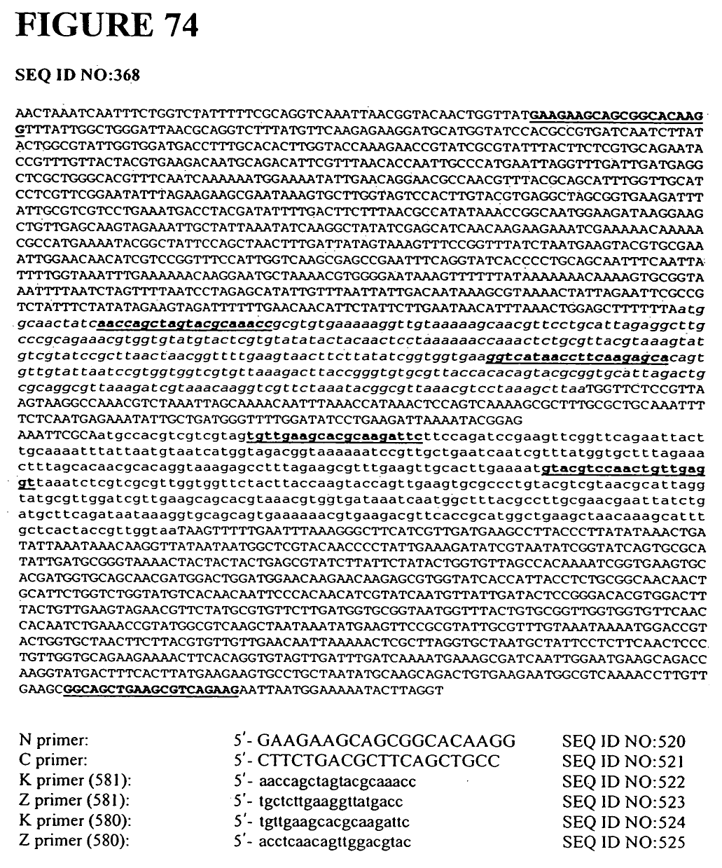

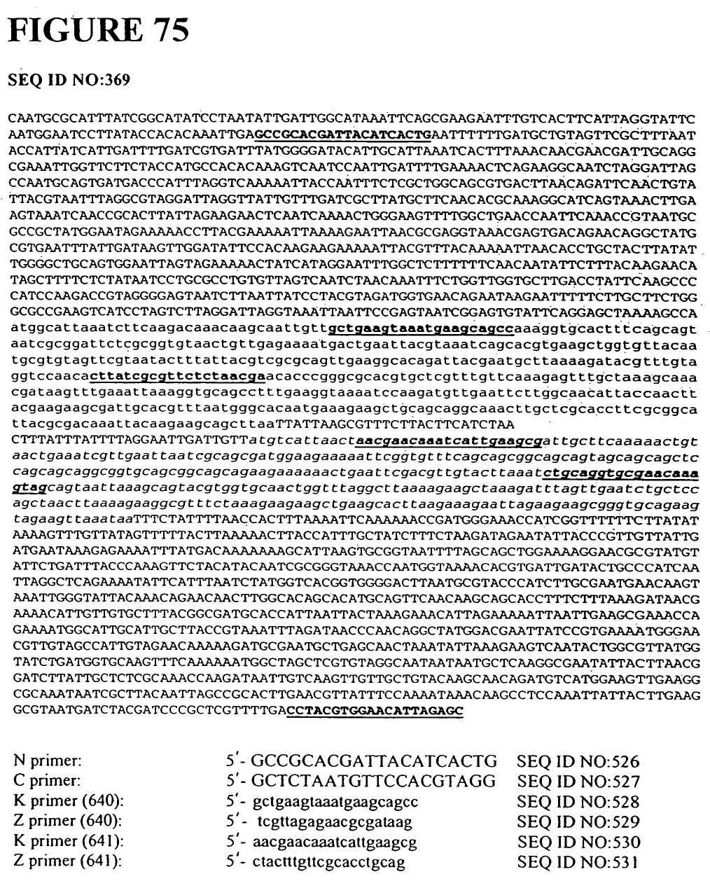

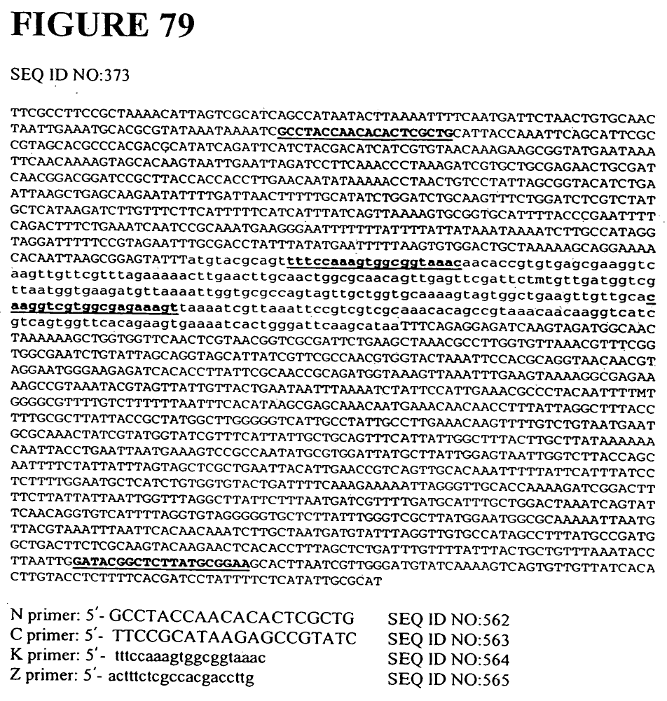

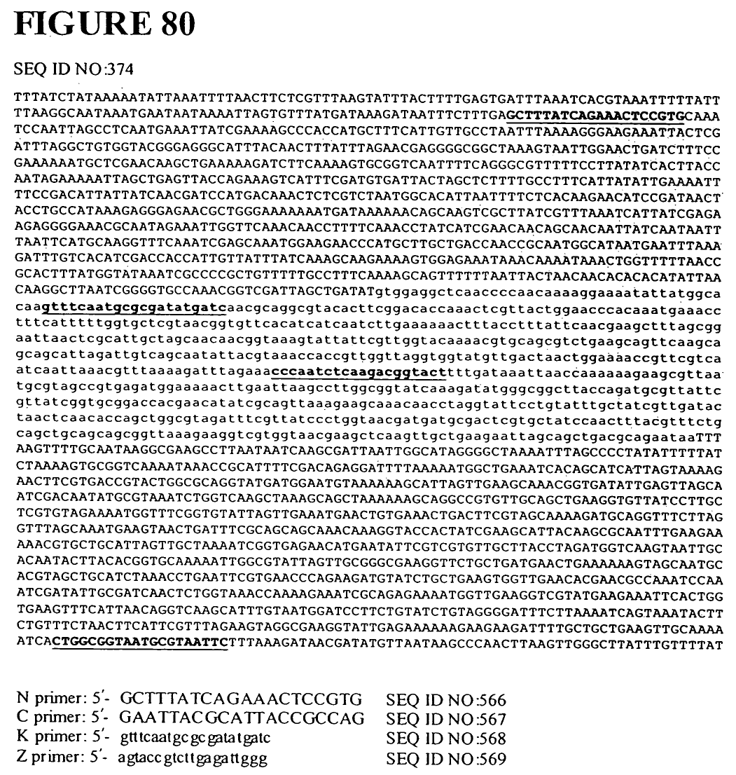

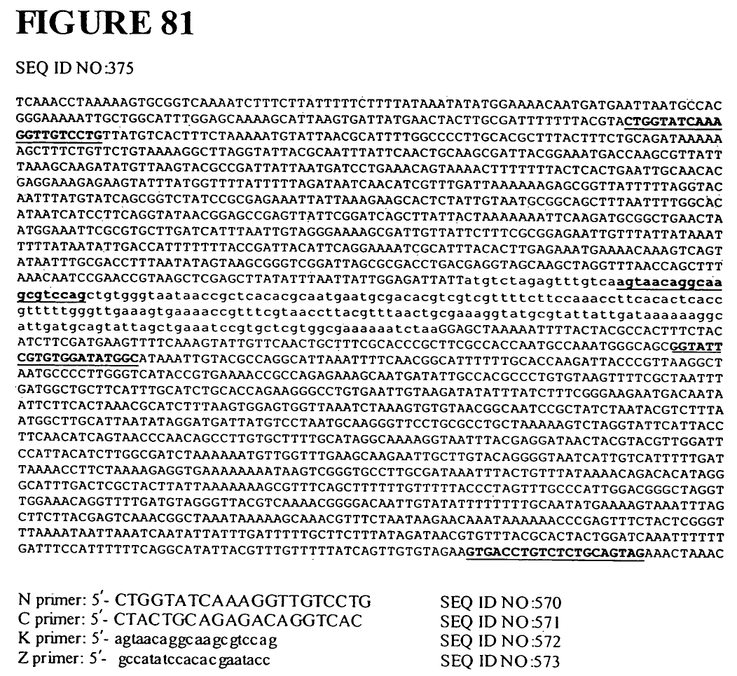

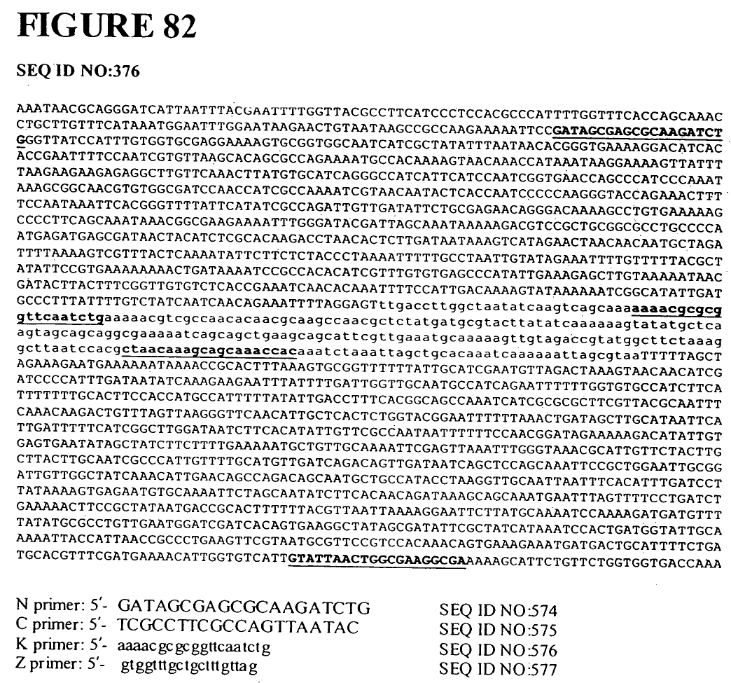

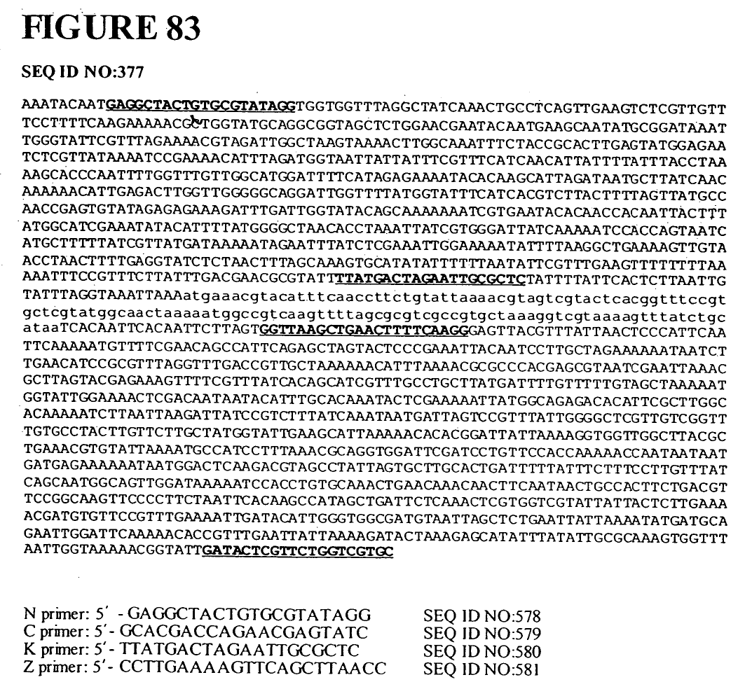

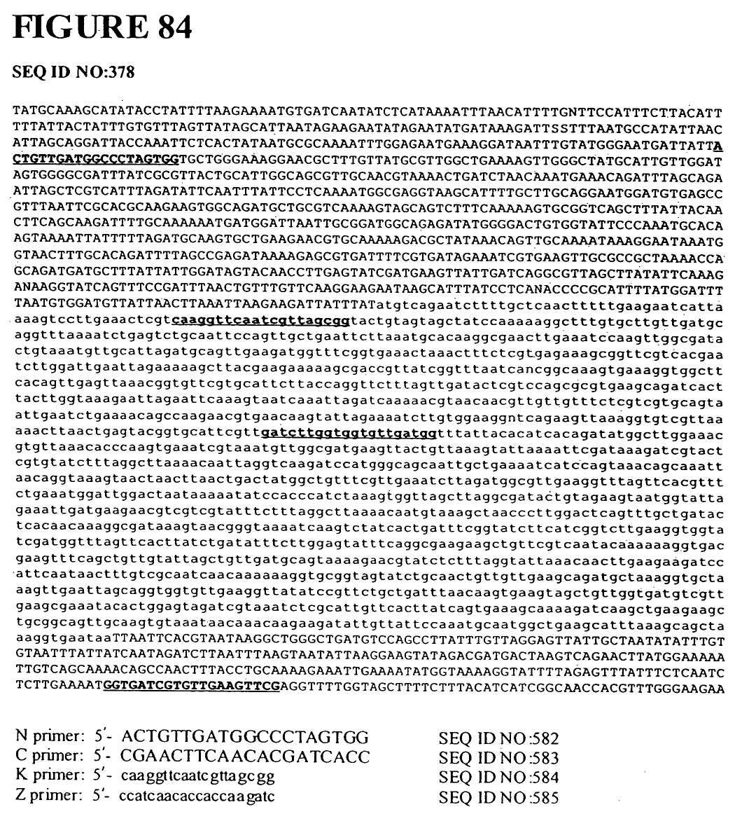

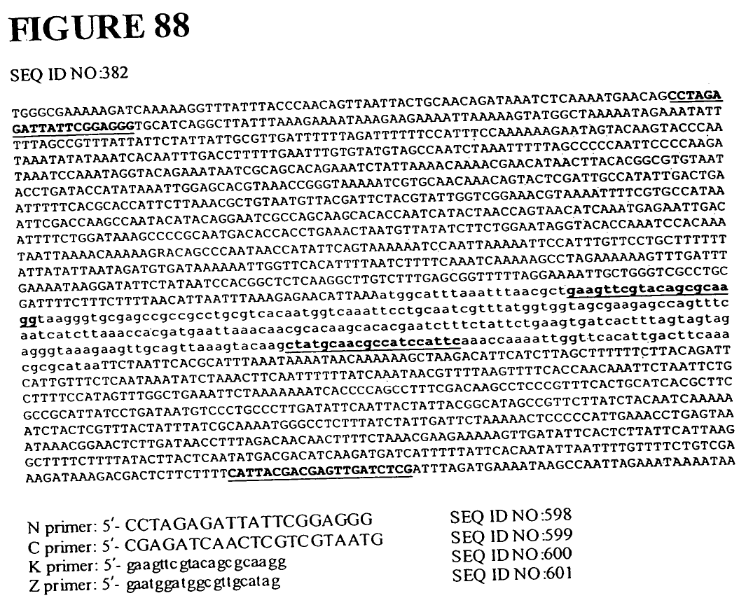

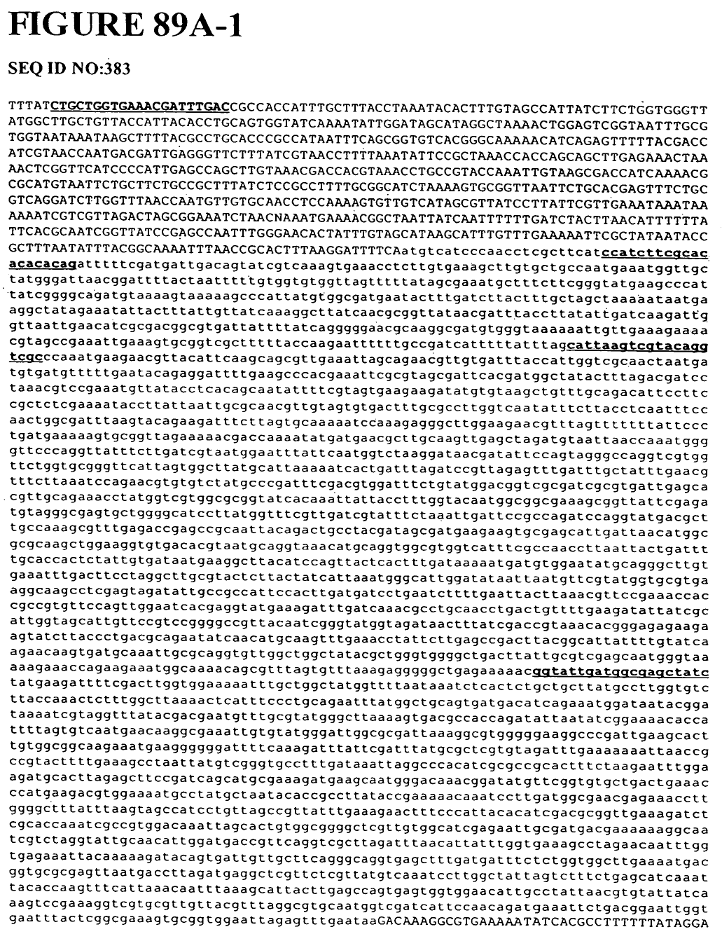

All of the gene sequences, flanking regions and PCR primer pairs are shown in FIGS. 70-88. For each gene or group of genes, the PCR product was synthesized, mutagenized with the EZ::Tn <Kan-2> transposon, and transformed directly into H. influenzae. Kanamycin selected recombinants were then screened with their essential region K/Z primer pair. In Figures in which more than one K/Z primer pair is represented, the numbers in parenthesis after each K or Z primer designates the last three digits of the gene for which the primer is specific. For example, in FIG. 70, “K primer (204)” indicates this particular K primer is specific for HI0204 and “K primer (201)” indicates this particular K primer is specific for HI0210.

Twenty-one of the twenty-two 30S ribosomal genes were found to be essential. These results are summarized in Table 5. The only gene determined to be non-essential was a duplicate copy of the rpsO gene HI1468(S15). Five of the 33 genes associated with the 50S ribosome were insertionally inactivated: rplI, rpmA, rpmE, rpmF and rpmG. The remaining 28 genes were all essential. These results are summarized in Table 7. The three genes associated with ribosome assembly rimK, rimL and prmA were all insertionally inactivated; therefore all three genes are non-essential. These results are summarized in Table 6. These three strains have been designated HI11604623 (rimK::Tn), HI111633 (rimL::Tn), and HI11036389 (prmA::Tn).

To confirm the non-essentiality of the five 50S ribosomal subunit genes rplI, rpmA, rpmE, rpmF, and rpmG, the five genes were individually deleted by homologous recombination of a PCR product containing the chloramphenicol resistance gene substituted for the targeted gene. The five deletion strains have been designated HI0544D (rplI), HI0879D (rpmA), HI0758D (rpmE), HI0158D (rpmF) and HI0950D (rpmG). In addition, an attempt was made to construct double mutant strains of the 5 non-essential 50S ribosomal genes in all possible combinations. Three double mutants were successfully constructed. Strain HI1158544 contains a deletion of the rpmF gene and an insertional inactivation of the rplI gene. Strain HI158758 contains a deletion of the rpmF gene and an insertional inactivation of the rpmE gene. Strain HI758950 contains a deletion of the rpmE gene and an insertional inactivation of the rpmG gene.

As shown in Tables 5-7, we have determined that 9 of the 58 H. influenzae ribosomal genes are non-essential. This is in contrast to E. coli, where 18 of the 58 ribosomal genes are non-essential. These include rpS6, rpS9, rpS13, rpS17, rpS20, rpL1, rpL9, rpL11, rpL15, rpL19, rpL24, rpL27, rpL28, rpL29, rpL30, rpL33, and the 3 genes associated with ribosome assembly rimK, rimL and prmA (Table 8). Two genes that are essential in E. coli were shown in this report to be non-essential in H. influenzae: rplI (rpL9) and rpmF (rpL32). These results illustrate the importance of determining gene essentiality in a variety of organisms when selecting potential targets for broad spectrum antibiotic development as individual organisms can require different genes for bacterial survival.

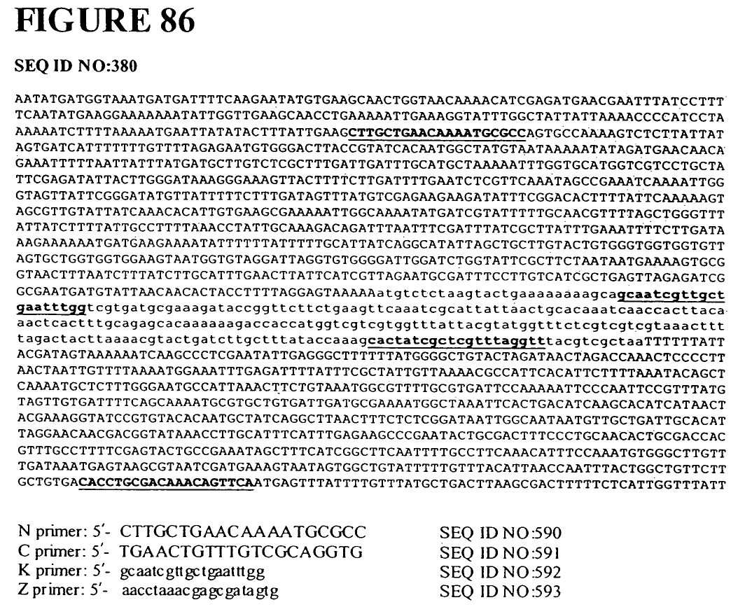

A search of the H. influenzae genomic database for the ribosomal proteins revealed two genes encoding for the S15 ribosomal protein rpsO: HI1328 (see FIG. 86) and HI1468. A direct sequence comparison showed that both genes were identical at the nucleotide level, suggesting that a chromosomal duplication had occurred. A similar gene duplication was identified during the essential determination of the cell wall biosynthetic genes in Example 4, where identical mrsA genes were encoded by both HI1337 and HI1463. Insertional inactivation followed by the attempted deletion of the remaining mrsA gene showed that although each gene could be independently inactivated, mutants could not be constructed that inactivated both copies within the same cell. Therefore, the mrsA protein product itself is required for cell viability. Taking the same approach for rpsO, we tried to first insertionally inactivate HI1328 and HI1468. We readily isolated clones containing an insert in HI1468. However, we were unable to isolate any inserts in HI1328 suggesting that this gene is essential regardless of the presence of the duplicate copy. These results are shown in Table 7. A mutant was then constructed that contained a deletion of HI1468, but we were unable to construct a mutant containing a deletion of HI1328. Taken together, these results show that HI1468 is non-essential whereas HI1328 is essential.

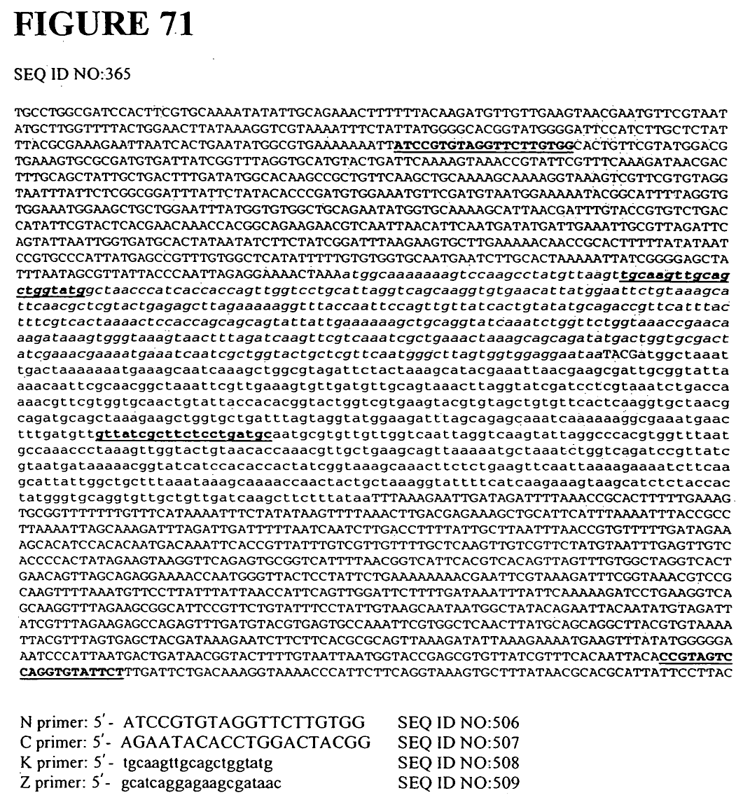

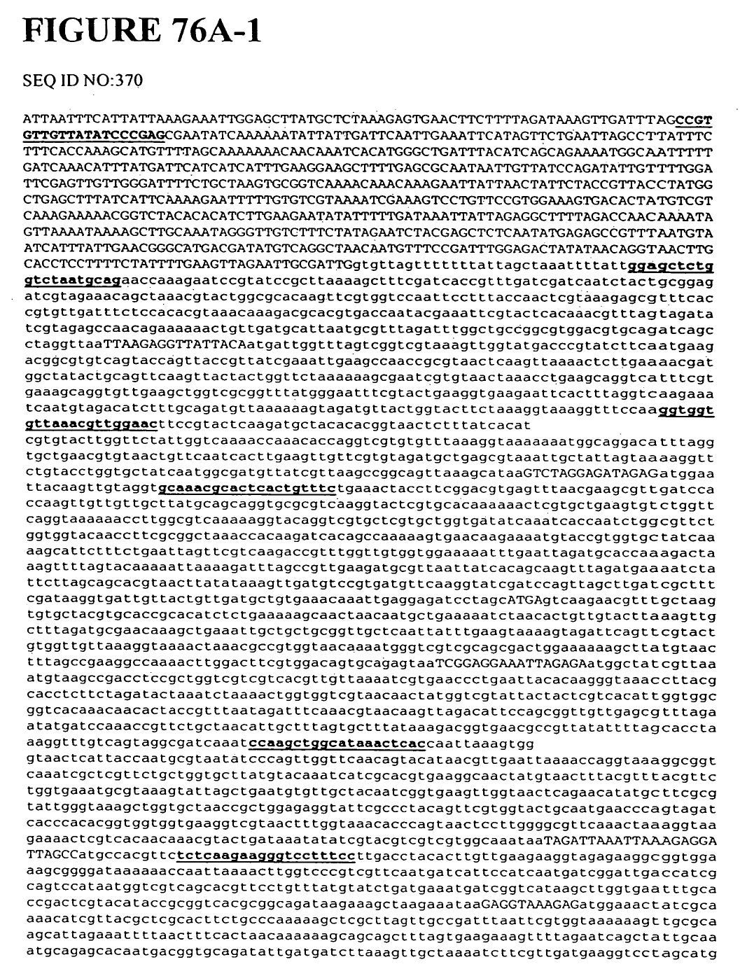

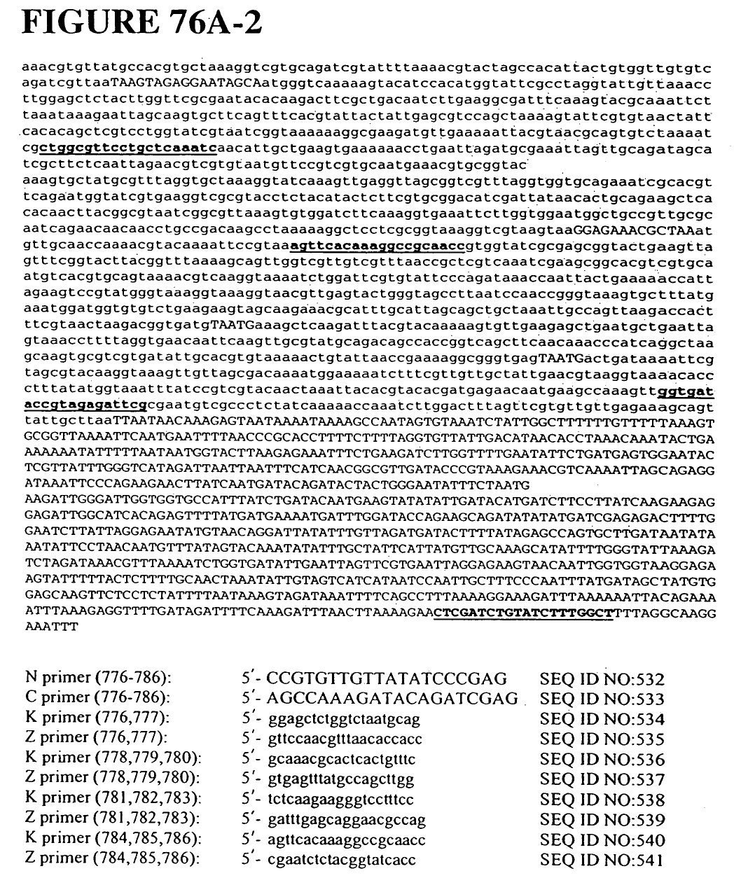

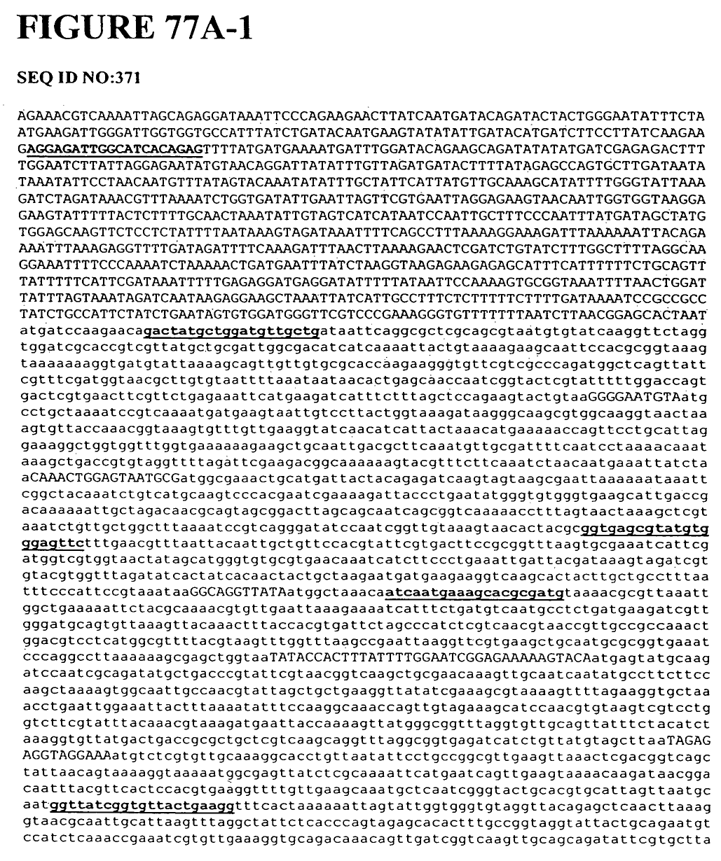

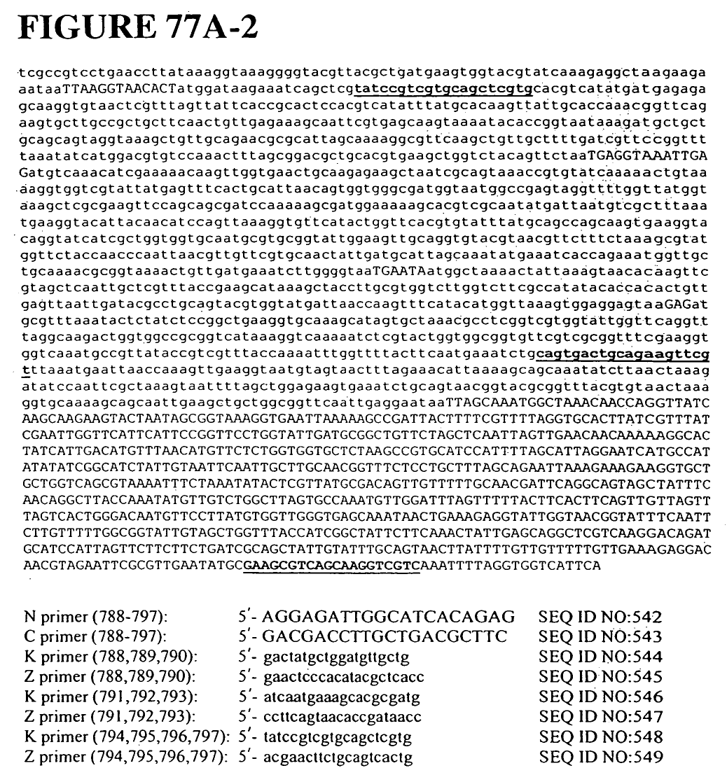

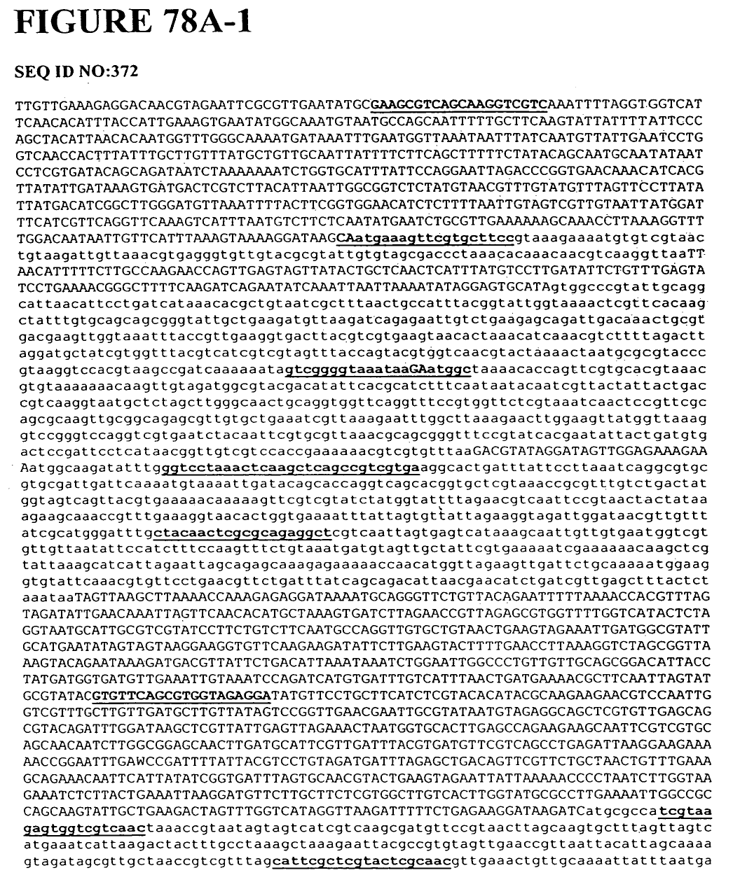

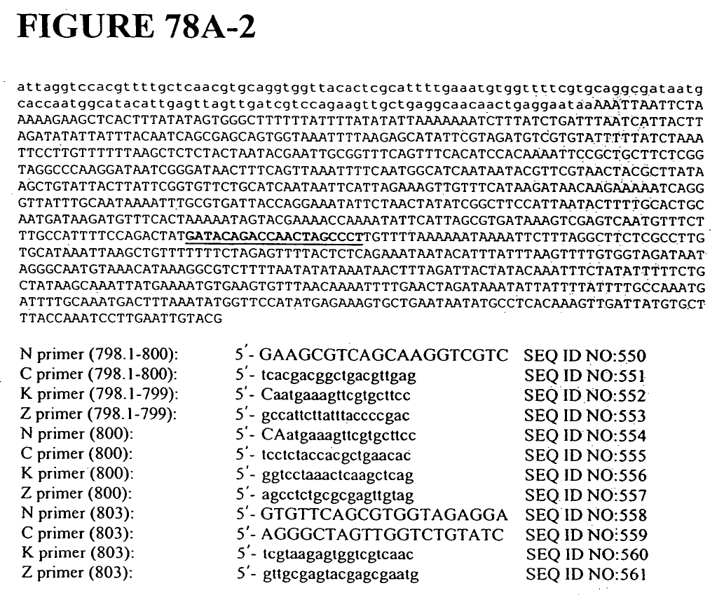

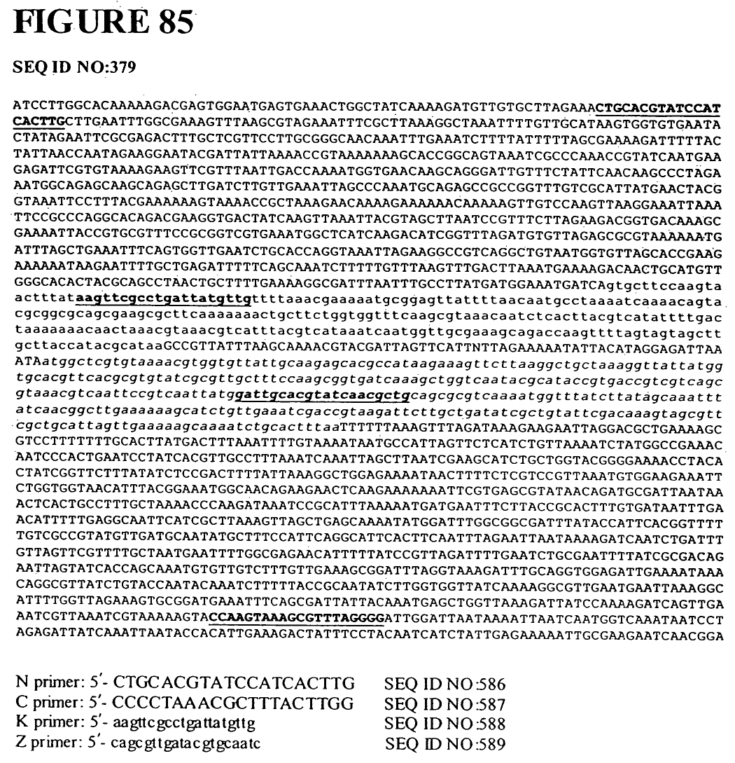

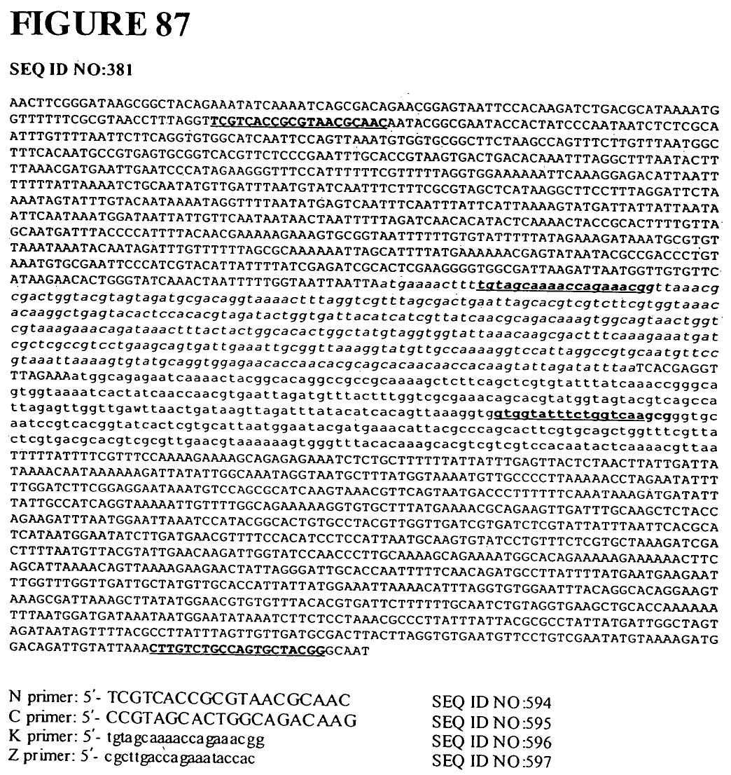

The described methodology details the use of a single K/Z primer pair encompassing the 5′ end of the target gene for screening purposes. Due to the small size (<200 basepairs) of many of the ribosomal genes and their proximity to adjacent ribosomal genes, a single K!Z primer pair was designed to encompass the 5′ region of one-target gene through the 5′ region of an adjacent target gene. The following sets of ribosomal genes were screened with a single K/Z primer pair as shown in the accompanying figures: HI0516/HI0517 (FIG. 71); HI0776/HI0777 (FIG. 76); HI0778/HI0779[HI0780 (FIG. 76); HI0781/HI0782/HI0783 (FIG. 76); HI0784/HI0785/HI0786 (FIG. 76); HI0788/HI0789/HI0790 (FIG. 77); HI0791/HI0792/HI0793 (FIG. 77); HI0794/HI0795/HI0796/HI0797 (FIG. 77); HI0798.1/HI0799 (FIG. 78); HI1319/HI1320 (FIG. 85); and HI1442/HI1443 (FIG. 87).

Insertional inactivation of the ribosomal genes presented a technical challenge in that 26 of the genes are tightly clustered in a 15 kb segment and have the same transcriptional directionality. It is likely that these genes are co-transcribed in a limited number of operons. The established protocol for determining essentiality requires screening recombinants with the K/Z primer pair, followed by re-screening with the N/C primer pair for inserts in the flanking regions if no inserts are found in the 5′ essential region. However within this extended ribosomal operon the flanking genes are also expected to be essential, so one would not expect to find inserts within the entire mutagenized PCR fragment. Distinguishing between failed mutagenesis and true essentiality relied on amplifying large fragments containing multiple genes flanked by at least one region not containing any ribosomal gene and detecting inserts within this one flanking region. Genes HI0776-HI0786 were synthesized as a single 6.6 kb fragment with inserts detected in the N-terminal flanking region. Genes HI0788-HI0797 were also synthesized as a single 5.6 kb fragment with inserts again detected in the N-terminal region. Due to the large size of these mutagenized fragments in proportion to the K/Z fragment length for each individual targeted gene, 192 recombinant colonies were screened with the KJZ primer pair per gene as opposed to the standard 96 colonies.

Repeated attempts at synthesizing and mutagenizing the last five genes in this large operon, HI0798.1, HI0799, HI0800, HI0801 and HI0803, failed to detect inserts in the flanking regions. To determine the essentiality of these five genes, we used an alternate method of vector mutagenesis. With this technique, the target genes are synthesized individually as a PCR fragment, cloned into an E. coli vector and mutagenized with the EZ::Tn <KAN-2> transposon. Following transformation, individual colonies are screened with the target gene K/Z primer pair to identify vectors containing Tn inserts within this gene. Vector DNA is then isolated and used for PCR amplification with the original N/C primer pair to produce an amplicon containing a known insertion within the target gene. This fragment is then transformed into H. influenzae and kanamycin selected recombinants are screened by PCR with the K/Z primer pair for the presence of the original Tn insert. If no inserts are detected even though each transformed amplicon contained a Tn insert within the target gene sequence, then the gene is determined to be essential.

The target gene with flanking sequences was PCR amplified from 1 mg of H. influenzae genomic DNA with the N/C primer set following the Platinum Taq™ amplification protocol: 94° C. for 5 min (1 cycle); 94° C. for 30 sec, 55° C. for 1 min and 72° C. for 3 min (30 cycles); and 72° C. for 5 min (1 cycle). The resulting amplicon was cloned into the pScript vector following the enclosed directions of the Stratagene cloning kit and transformed into competent E. coli InvαF′ cells purchased from Invitrogen. Colonies were screened by restriction analysis to confirm the presence of the PCR insert. Vector DNA was isolated using columns purchased from Qiagen.

Following the package directions of the Epicentre EZ::Tn™<KAN-2> insertion kit, 100 ng of the vector was mutagenized for 2 hours at 37° C. After heat inactivation of the transposase at 70° C. for 10 minutes, 20 μl of sterile water was then added and the reaction was passed through a Millipore Ultrafree column to remove the enzyme. 20 ng of vector was then transformed directly into E. coli InvαF′ cells. Transformants were selected on Luria broth plates containing 30 μg/ml kanamycin. 48 colonies were screened with the respective K/Z primer pair to identify a vector containing a single insertion within each target gene. Vector DNA was isolated and the respective N/C primer pair was used to amplify the target gene containing the Tn insert within the gene. 100 ng of the PCR product was transformed into competent cells of H. influenzae and plated onto sBHI supplemented with 30 μg/ml kanamycin. After 48 hours of incubation at 37° C., all resulting colonies were screened with their respective K/Z primer pair to detect Tn inserts within the targeted gene. None were found in any of these five genes. Therefore, we concluded that HI0798.1, HI0799, HI0800, HI0801 and HI0803 are essential.

Ribosomal gene essentiality determined by the targeted gene disruption method of the present invention compared to ribosomal gene essentiality determined using a whole-genome approach reported by Akerley et al. (Proc. Natl. Acad. Sci. USA 99(2), 966-971 (2002)) is shown in Table 9. Thirty-nine genes were found to be essential and two genes were found to be non-essential by both methods. The results differed on six genes: rplL, rpsO (HI1468), rimI, rimK, prmA, and rpmE. The essentiality of the remaining 12 genes was not determined by the whole-genome approach. There are several benefits to identifying essential genes by targeted analysis. This approach generates a mutant library of individual clones that can be sequenced to determine the exact transposon insertion site, whereas results from genomic approaches are derived from mutant pools. The resulting individual clones are also available for further genetic analysis. Small genes (<300 bp) can be directly evaluated without relying on their proximity to anchoring PCR primers for accurate gel mapping. Targeted analysis also identifies insertionally inactivated genes whose protein product loss has a negative effect on the growth rate of the cell. The targeted approach is able to address the essentiality of cellular function when two or more genes encode f6r the same protein by using transposons with different selectable markers or by using insertional inactivation in combination. with gene deletion (i.e. rpsO encoded by both HI1328 and HI1468). Targeted analysis does sacrifice the overall speed of the genomic approach. However, the gains made in the accuracy of the data can offset the additional time required for essential determination.

Expanding on the above comparison of gene essentiality determined the targeted gene disruption method of the present invention to gene essentiality determined using the whole-genome approach reported by Akerley et al., the following genes were found to be essential by both methods: aroC, coaD, yrdC, IF-1, IF-2, IF-3, pth, tmk, alr, ddlB, ftsI, murC, murD, murE, murF, murG, nagA, emrB, pyrH, rpsA, rpsB, rpsC, rpsD, rpsE, rpsF, rpsG, rpsH, rpsI, rpsK, rpsL, rpsM, rpsN, rpsP, rpsQ, rpsR, rpsS, rplB, rplD, rplE, rplF, rplJ, rplK, rplM, rplN, rplO, rplP, rplQ, rplR, rplS, rplT, rplV, rplW, rplX, rplY, rpmC, rpmD, rpmH, rpmI, and dnaE. The following genes were determined to be essential by the targeted gene disruption method of the present invention, but were determined to be non-essential by the whole-genome approach of Akerley: fba, fmt, uppS, pal, rplL, cdsA, and coaE. The following genes have been determined to be essential by the targeted gene disruption method of the present invention, while essentiality has not been determined using the whole-genome approach of Akerley: ribB, ribF, nusA, glmU, mraY, murB, murZ, murI, rpS15 (HI1328), rpsJ, rpsT, rpsU, rplA, rplC, rplU, rpmB, rpmJ, and mesJ.

The comparison of non-essential ribosomal genes between E. coli and H. influenzae (Table 8) reveals many differences f6r these two closely related bacteria. Although ribosomal RNA and ribosomal proteins retain strong homology across species, accumulated subtle changes in the ribosome can collectively engender significant macromolecular structural differences in the ribosomes from different bacterial species.

Targeted gene disruption was used to determine the essentiality of the 58 ribosomal genes in H. influenzae. Forty-nine genes were found to be essential. The remaining 9 genes were found to be non-essential: rimI, rimK, prmA, rplI (rpL9), rpmA (rpL27), rpmE (rpL31), rpmF (rpL32), rpmG (rpL33), and the duplicate copy of rpsO (rpS15) encoded by HI1468.

(rpS)

TABLE 5
Essentiality results of the 30S ribosomal subunit genes

| | SEQ | | | | | |
|---|---|---|---|---|---|---|
| | ID | | SEQ ID NO: | | | No. of |
| Gene* | NO: | Locus | polypeptide | Function | Insertions | inserts |
| rpsA (rpS1) | 486 | HI1220 | 431 | 30S ribosomal subunit S1 | − | 0/96 |
| rpsB (rpS2) | 482 | HI0913 | 427 | 30S ribosomal subunit S2 | − | 0/96 |
| rpsC (rpS3) | 462 | HI0783 | 407 | 30S ribosomal subunit S3 | − | 0/96 |
| rpsD (rpS4) | 479 | HI0801 | 424 | 30S ribosomal subunit S4 | − | 0/10 |
| rpsE (rpS5) | 473 | HI0795 | 418 | 30S ribosomal subunit S5 | − | 0/96 |
| rpsF (rpS6) | 450 | HI0547 | 395 | 30S ribosomal subunit S6 | − | 0/96 |
| rpsG (rpS7) | 451 | HI0580 | 396 | 30S ribosomal subunit S7 | − | 0/96 |
| rpsH (rpS8) | 470 | HI0792 | 415 | 30S ribosomal subunit S8 | − | 0/96 |
| rpsI (rpS9) | 490 | HI1442 | 435 | 30S ribosomal subunit S9 | − | 0/96 |
| rpsJ (rpS10) | 455 | HI0776 | 400 | 30S ribosomal subunit S10 | − | 0/192 |
| rpsK (rpS11) | 478 | HI0800 | 423 | 30S ribosomal subunit S11 | − | 0/19 |
| rpsL (rpS12) | 452 | HI0581 | 397 | 30S ribosomal subunit S12 | − | 0/96 |
| rpsM (rpS13) | 477 | HI0799 | 422 | 30S ribosomal subunit S13 | − | 0/8 |
| rpsN (rpS14) | 469 | HI0791 | 414 | 30S ribosomal subunit S14 | − | 0/96 |
| rpsO (rpS15) | 489 | HI1328 | 434 | 30S ribosomal subunit S15 | − | 0/96 |
| rpsO (rpS15) | — | HI1468 | — | 30S ribosomal subunit S15 | + | 30/96 |
| rpsP (rpS16) | 445 | HI0204 | 390 | 30S ribosomal subunit S16 | − | 0/96 |
| rpsQ (rpS17) | 465 | HI0786 | 410 | 30S ribosomal subunit S17 | − | 0/192 |
| rpsR (rpS18) | 449 | HI0545 | 394 | 30S ribosomal subunit S18 | − | 0/96 |
| rpsS (rpS19) | 460 | HI0781 | 405 | 30S ribosomal subunit S19 | − | 0/192 |
| rpsT (rpS20) | 484 | HI0965 | 429 | 30S ribosomal subunit S20 | − | 0/96 |
| rpsU (rpS21) | 448 | HI0531 | 393 | 30S ribosomal subunit S21 | − | 0/96 |

The insertional inactivation results of the 22 genes associated with the 30S ribosomal unit. Twenty-one genes were determined to be essential; the only non-essential gene is the duplicate copy of the rspO HI1468.
*Alternative gene name is given in parenthesis.

TABLE 6
Essentiality results of associated ribosomal synthetic proteins

| | | | | No. of |
|---|---|---|---|---|
| Gene | Locus | Function | Insertions | inserts |
| rimK | HI1531 | 50S protein S6 modification | + | 8/48 |
| | | protein | | |
| rimI | HI0010 | ribosomal protein alanine | + | 6/48 |
| | | transferase | | |
| prmA | HI0879 | ribosomal protein L11 | + | 7/48 |
| | | methyltransferase | | |

The insertional inactivation results of the three proteins associated with ribosome synthesis. All three genes were determined to be non-essential.

TABLE 7
Essentiality results of the 50S ribosomal subunit genes

| | SEQ ID | | SEQ ID NO: | | | No. of |
|---|---|---|---|---|---|---|
| Gene | NO: | Locus | polypeptide | Function | Insertions | inserts |
| rplA (rpL1) | 446 | HI0516 | 391 | 50 S ribosomal protein L1 | − | 0/96 |
| rplB (rpL2) | 459 | HI0780 | 404 | 50 S ribosomal protein L2 | − | 0/192 |
| rplC (rpL3) | 456 | HI0777 | 401 | 50 S ribosomal protein L3 | − | 0/192 |
| rplD (rpL4) | 457 | HI0778 | 402 | 50 S ribosomal protein L4 | − | 0/192 |
| rplE (rpL5) | 468 | HI0790 | 413 | 50 S ribosomal protein L5 | − | 0/96 |
| rplF (rpL6) | 471 | HI0793 | 416 | 50 S ribosomal protein L6 | − | 0/96 |
| rplL (rpL7/L12) | 454 | HI0641 | 399 | 50 S ribosomal protein | − | 0/96 |
| | | | | L7/12 | | |
| rplI (rpL9) | — | HI0544 | — | 50 S ribosomal protein L9 | + | 8/48 |
| rplJ (rpL10) | 453 | HI0640 | 398 | 50 S ribosomal protein L10 | − | 0/96 |
| rplK (rpL11) | 447 | HI0517 | 392 | 50 S ribosomal protein L11 | − | 0/96 |
| rplM (rpL13) | 491 | HI1443 | 436 | 50 S ribosomal protein L13 | − | 0/96 |
| rplN (rpL14) | 466 | HI0788 | 411 | 50 S ribosomal protein L14 | − | 0/96 |
| rplO (rpL15) | 475 | HI0797 | 420 | 50 S ribosomal protein L15 | − | 0/96 |
| rplP (rpL16) | 463 | HI0784 | 408 | 50 S ribosomal protein L16 | − | 0/192 |
| rplQ (rpL17) | 480 | HI0803 | 425 | 50 S ribosomal protein L17 | − | 0/14 |
| rplR (rpL18) | 472 | HI0794 | 417 | 50 S ribosomal protein L18 | − | 0/96 |
| rplS (rpL19) | 444 | HI0201 | 389 | 50 S ribosomal protein L19 | − | 0/96 |
| rplT (rpL20) | 488 | HI1320 | 433 | 50 S ribosomal protein L20 | − | 0/96 |
| rplU (rpL21) | 481 | HI0880 | 426 | 50 S ribosomal protein L21 | − | 0/96 |
| rplV (rpL22) | 461 | HI0782 | 406 | 50 S ribosomal protein L22 | − | 0/192 |
| rplW (rpL23) | 458 | HI0779 | 403 | 50 S ribosomal protein L23 | − | 0/192 |
| rplX (rpL24) | 467 | HI0789 | 412 | 50 S ribosomal protein L24 | − | 0/96 |
| rplY (rpL25) | 492 | HI1630 | 437 | 50 S ribosomal protein L25 | − | 0/96 |
| rpmA (rpL27) | — | HI0879 | — | 50 S ribosomal protein L27 | + | 13/96 |
| rpmB (rpL28) | 483 | HI0951 | 428 | 50 S ribosomal protein L28 | − | 0/96 |
| rpmC (rpL29) | 464 | HI0785 | 409 | 50 S ribosomal protein L29 | − | 0/192 |
| rpmD (rpL30) | 474 | HI0796 | 419 | 50 S ribosomal protein L30 | − | 0/96 |
| rpmE (rpL31) | — | HI0758 | — | 50 S ribosomal protein L31 | + | 21/48 |
| rpmF (rpL32) | — | HI0158 | — | 50 S ribosomal protein L32 | + | 2/48 |
| rpmG (rpL33) | — | HI0950 | — | 50 S ribosomal protein L33 | + | 15/96 |
| rpmH (rpL34) | 485 | HI0998 | 430 | 50 S ribosomal protein L34 | − | 0/96 |
| rpmI (rpL35) | 487 | HI1319 | 432 | 50 S ribosomal protein L35 | − | 0/96 |
| rpmJ (rpL36) | 476 | HI0798.1 | 421 | 50 S ribosomal protein L36 | − | 0/96 |

The insertional inactivation results of the 33 proteins associated with the 50S ribosomal subunit. All but five were determined to be essential. Genes rplI, rpmA, rpmE, rpmF and rpmG are not essential.
*Alternative gene name is given in parenthesis.

TABLE 8
Comparison of the non-essential ribosomal genes of
H. influenzae and E. coli

| | Essential in | | |
|---|---|---|---|
| Gene | H. flu | Essential in E. coli | Reference |
| rpsF (rpS6)* | Yes | No | Dabbs¹ |
| rpsI (rpS9) | Yes | No | Dabbs² |
| rpsM (rpS13) | Yes | No | Dabbs¹ |
| rpsQ (rpS17) | Yes | No | Dabbs² |
| rpsT (rpS20) | Yes | No | Dabbs³ |
| rplA (rpL1) | Yes | No | Dabbs⁴ |
| rplI (rpL9) | No | Yes | This study |
| rplK (rpL11) | Yes | No | Stoffler⁵ |
| rplO (rpL15) | Yes | No | Lotti⁶ |
| rplS (rpL19) | Yes | No | Lotti⁶ |
| rplX (rpL24) | Yes | No | Herold⁷ |
| rpmA (rpL27) | No | No | Dabbs² |
| rpmB (rpL28) | Yes | No | Dabbs² |
| rpmC (rpL29) | Yes | No | Dabbs² |
| rpmD (rpL30) | Yes | No | Dabbs² |
| rpmE (rpL31) | No | Yes | This study |
| rpmF (rpL32) | No | Yes | This study |
| rpmG (rpL33) | No | No | Dabbs² |
| rimK | No | No | Kang et al.⁸ |
| rimI | No | No | Isono and Isono⁹ |
| prmA | No | No | Vanet et al.¹⁰ |

There are 9 non-essential genes in H. influenzae and 18 non-essential genes in E. coli.
*Alternative gene name is given in parenthesis.
¹Dabbs, Biochimie, 73: 639-645 (1991).
²Dabbs, J Bac, 140(2): 734-737 (1979).
³Dabbs, Mol Gen Genet, 192: 301-308 (1983).
⁴Dabbs, J Mol Biol, 149: 553-578 (1981).
⁵Stoffler et al., Mol Gen Genet, 181: 164-168 (1981).
⁶Lotti et al., Mol Gen Genet, 192: 295-300 (1983).
⁷Herold et al., Mol Gen Genet, 203: 281-287 (1986).
⁸Kang et al., Mol Gen Genet, 217: 281-288 (1989).
⁹Isono and Isono, Mol Gen Genet, 177: 645-651 (1980).
¹⁰Vanet et al., Mol Microbiol, 14(5): 947-958 (1994).

TABLE 9
Targeted gene disruption vs. whole genome analysis

| Targeted | Genomic | 58 Total | Genes |
|---|---|---|---|
| Essential | Essential | 39 | rpsA, rpsB, rpsC, rpsD, rpsE, |
| | | | rpsF, rpsG, rpsH, rpsI, rpsK, |
| | | | rpsL, rpsM rpsN, rpsP, rpsQ, |
| | | | rpsR, rpsS, rplB, rplD, rplE, |
| | | | rplF, rplJ, rplK, rplM, rplN, |
| | | | rplO, rplP, rplQ, rplR, rplS, |
| | | | rplT, rplV, rplW, rplX rplY, |
| | | | rpmC, rpmD, rpmH, rpmI |
| Non-essential | Non-essential | 1 | rplI |
| Essential | Non-essential | 1 | rplL |
| Non-essential | Essential | 5 | rpsO (HI1468), rimI, rimK, |
| | | | prmA, rpmE |
| Essential | Not | 9 | rpsO (HI1328), rpsJ, rpsT, |
| | Determined | | rpsU, rplA, rplC, rplU, rpmB, |
| | | | rpmJ |
| Non-essential | Not | 3 | rpmA, rpmF, rpmG |
| | Determined | | |

Comparison of ribosome essentiality as determined by targeted gene disruption verses whole-genome analysis.

### Example 6

### Evaluation of Essentiality of Additional H. influenzae Coding Sequences

This example demonstrates the essentiality of six additional H. influenzae coding sequence.

Materials and Methods

The materials and methods used are described in Example 1.

Results.

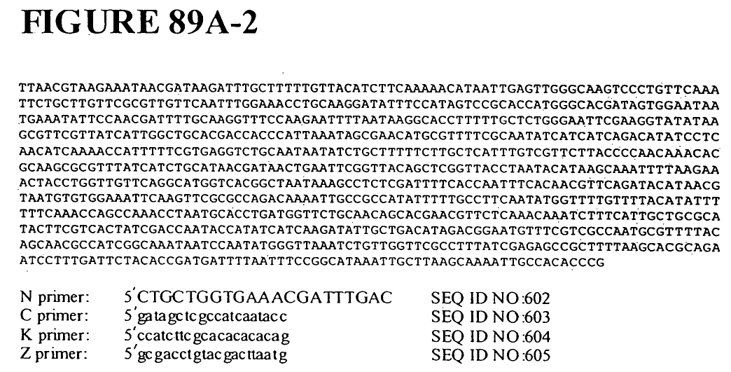

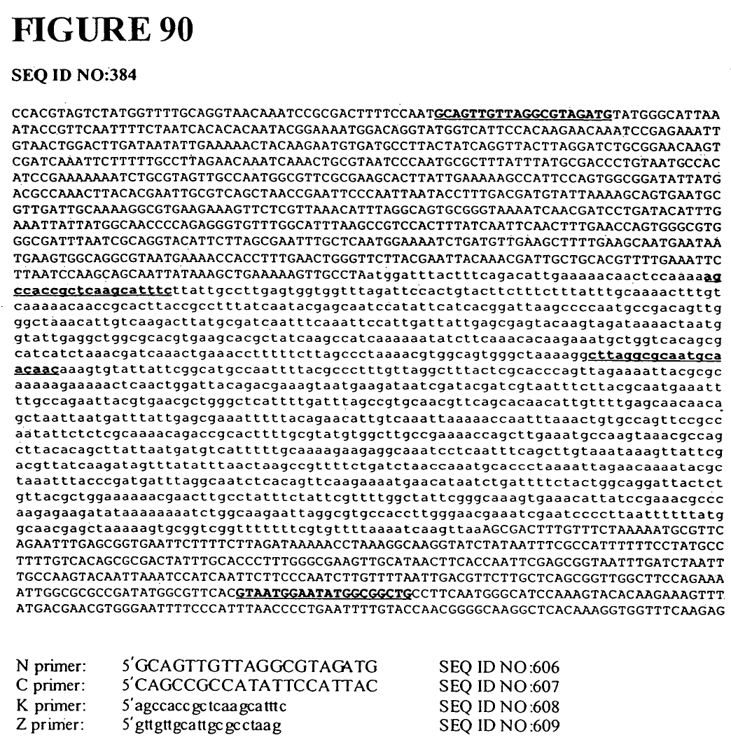

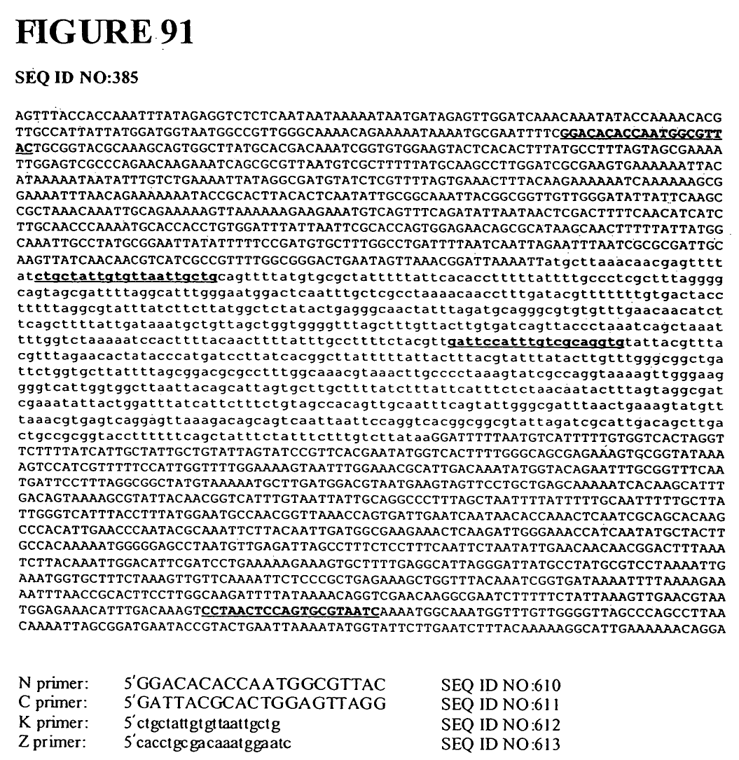

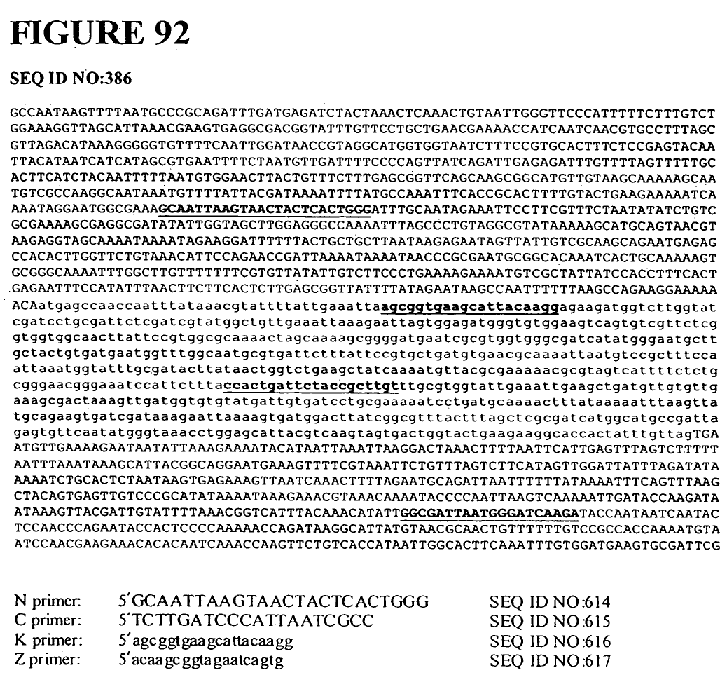

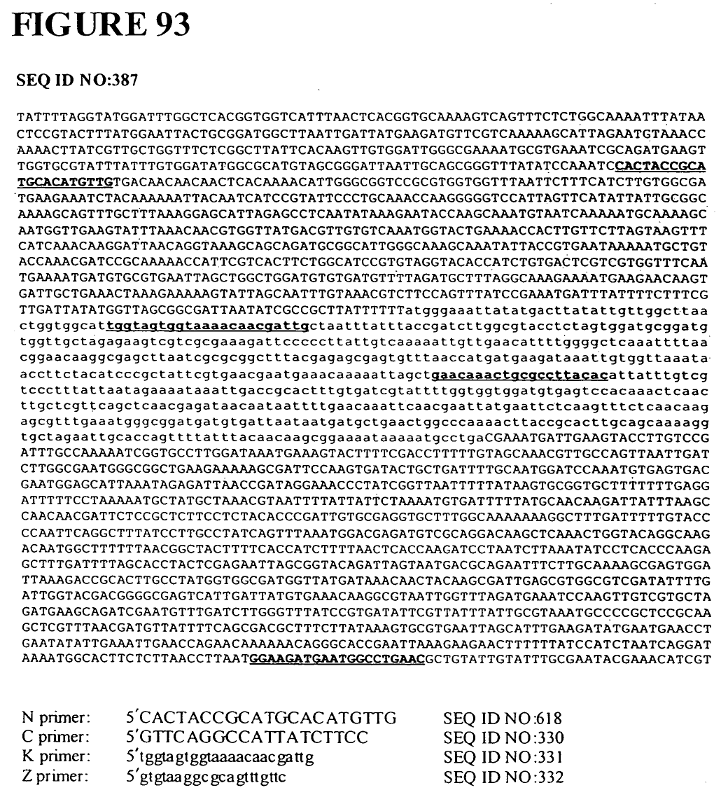

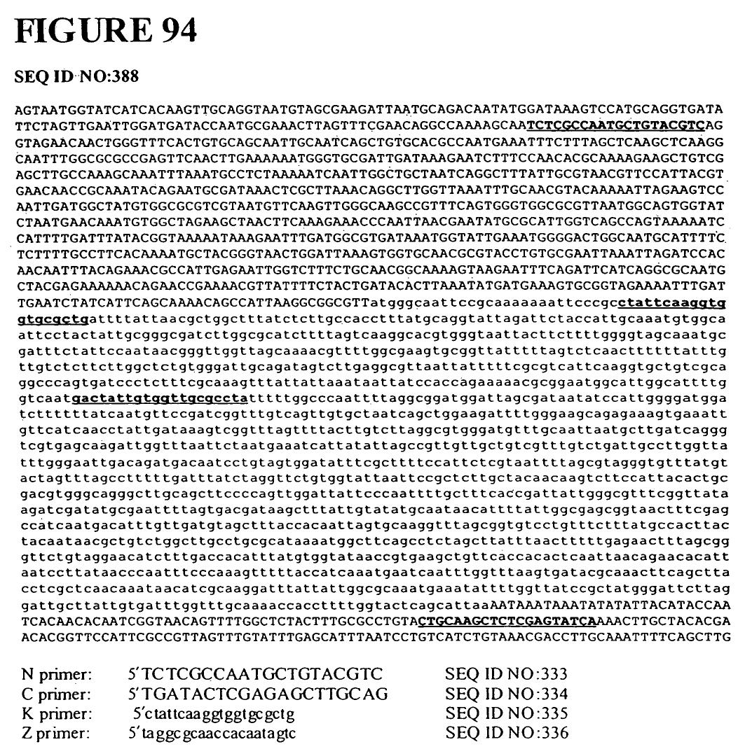

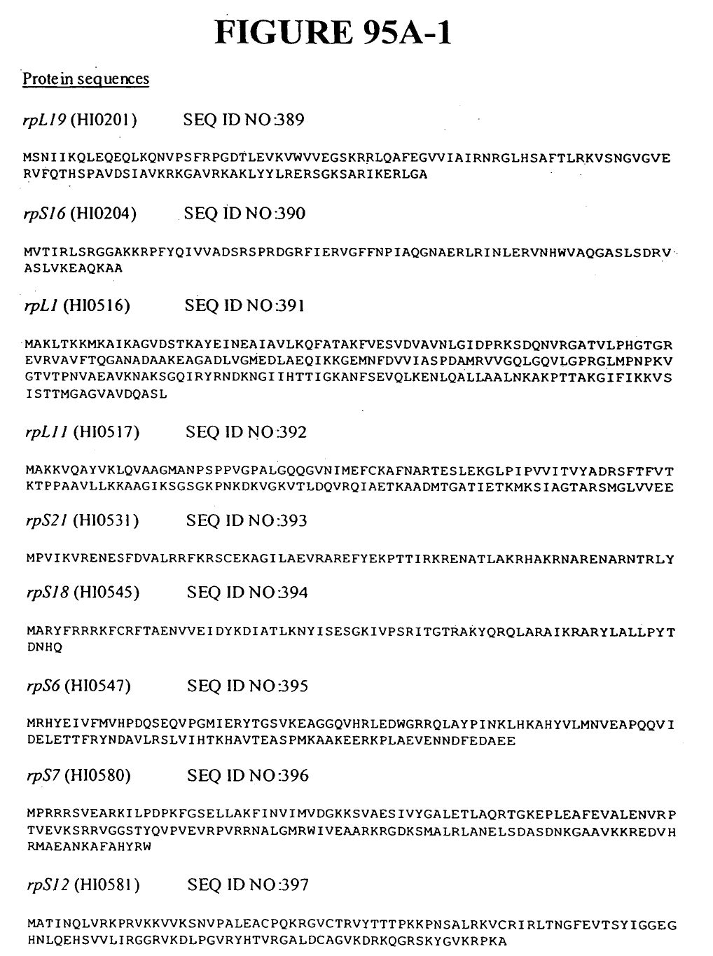

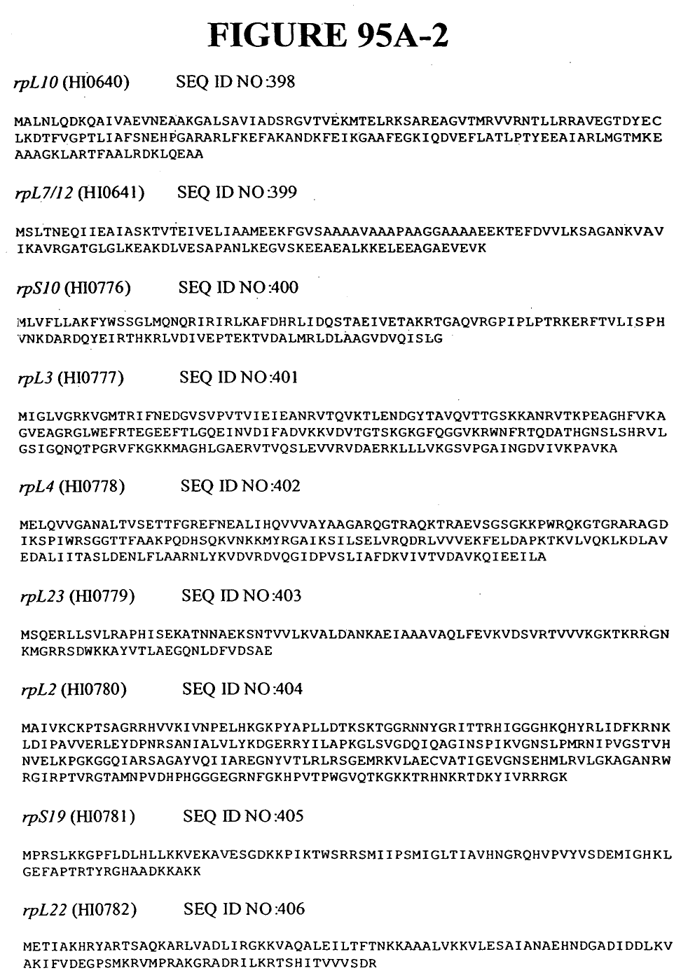

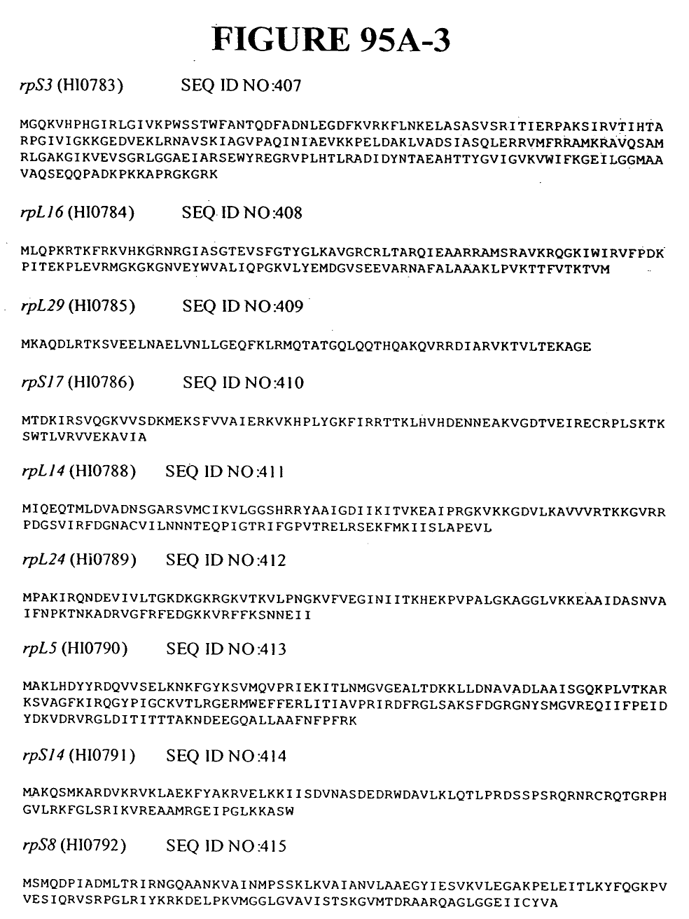

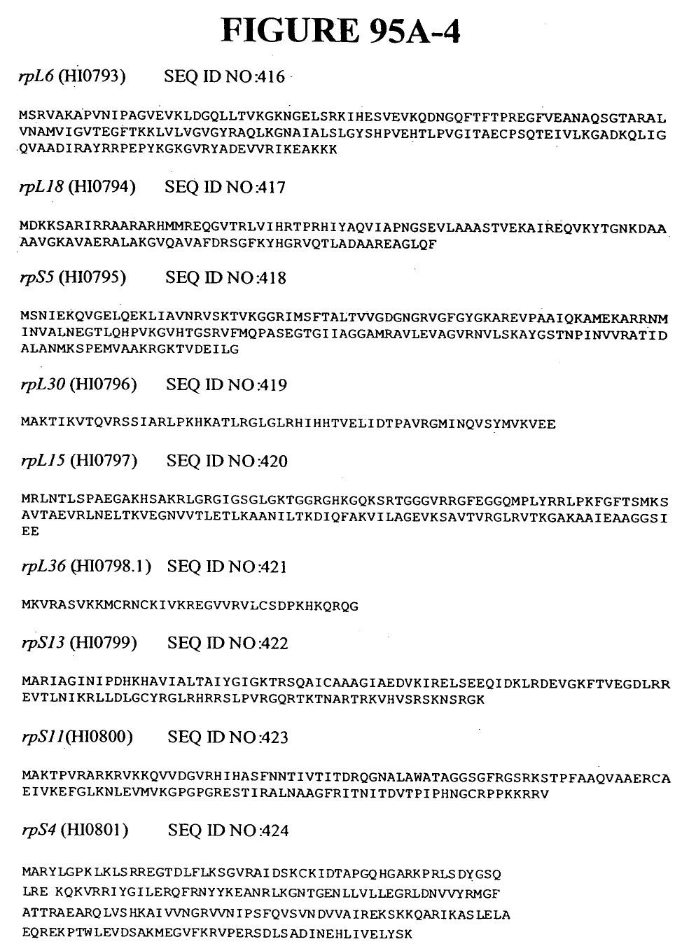

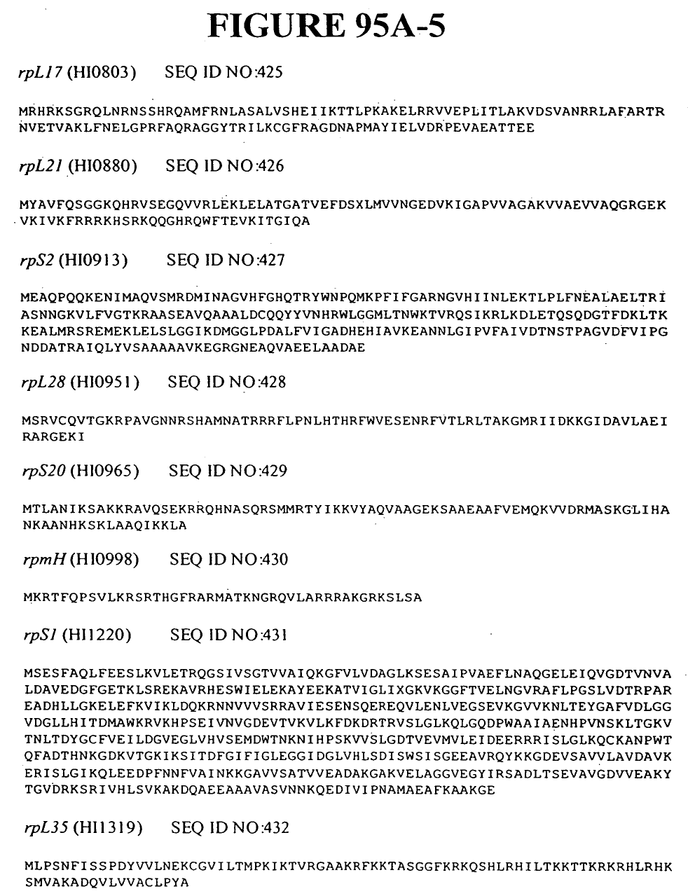

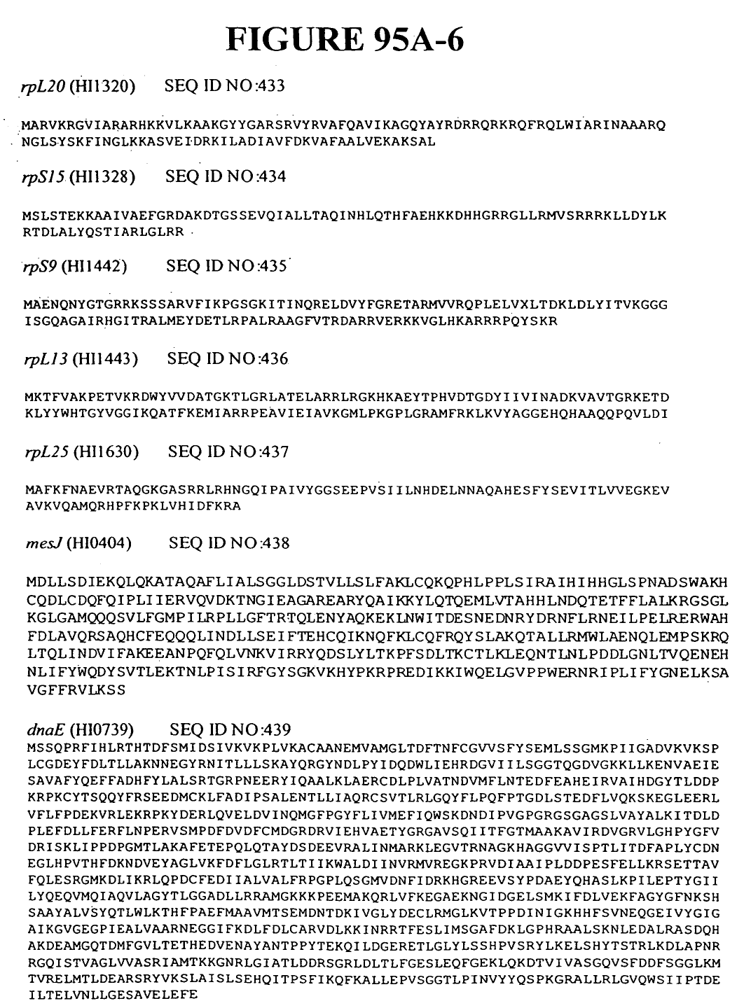

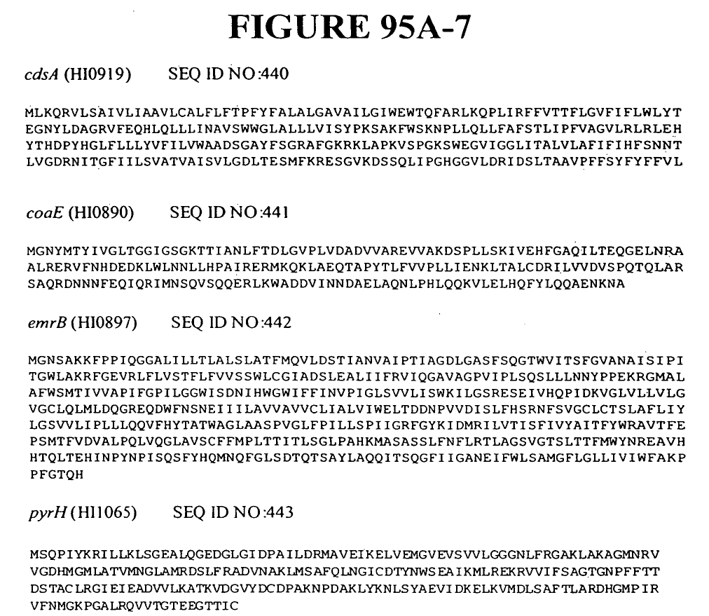

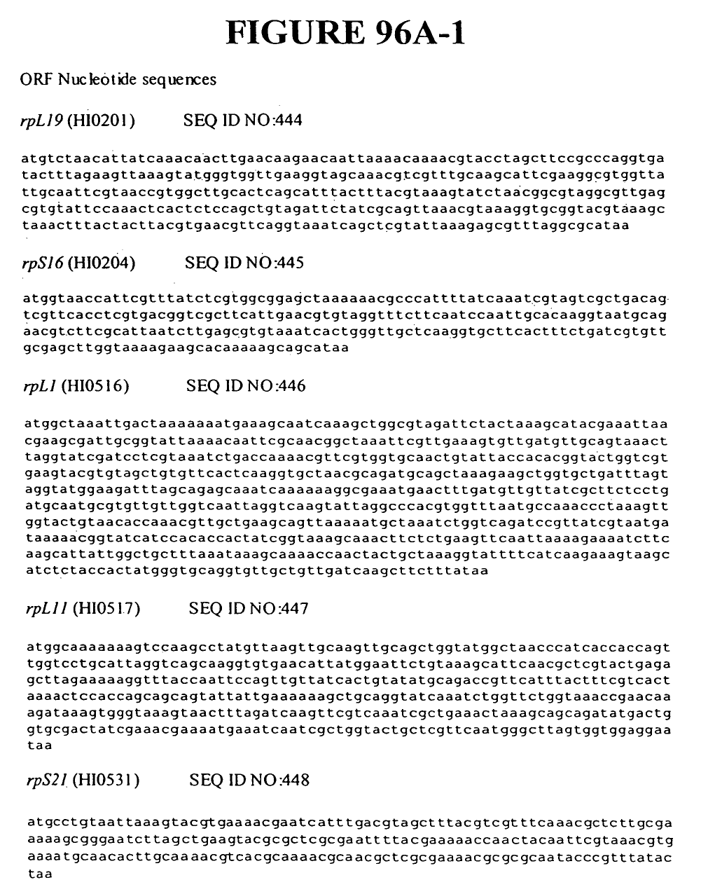

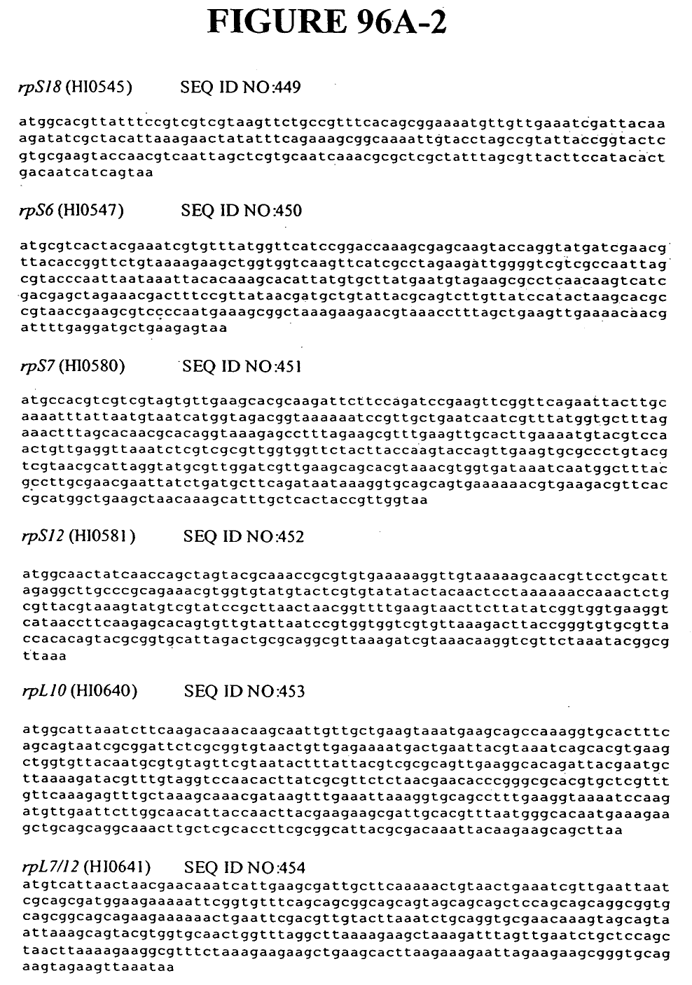

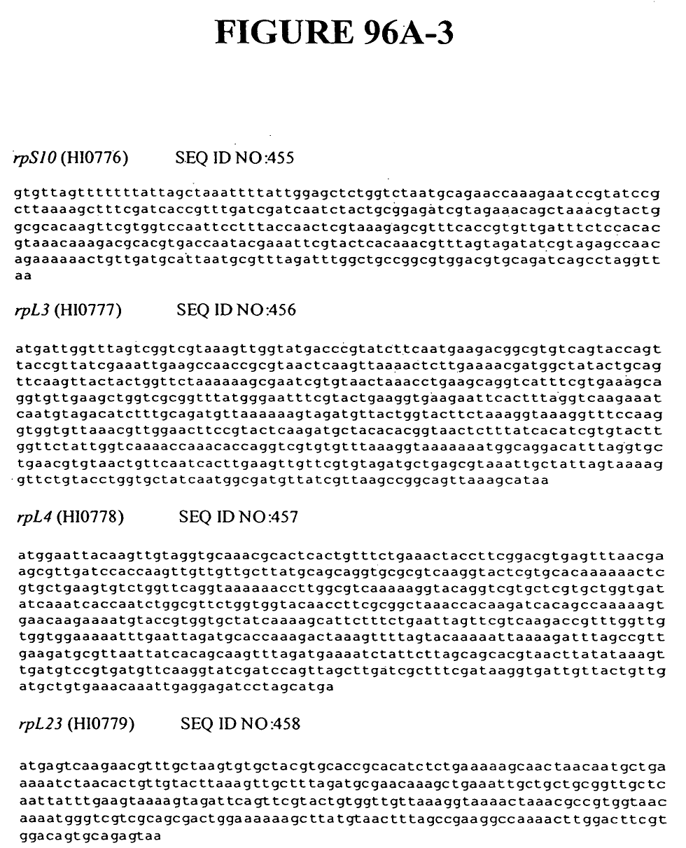

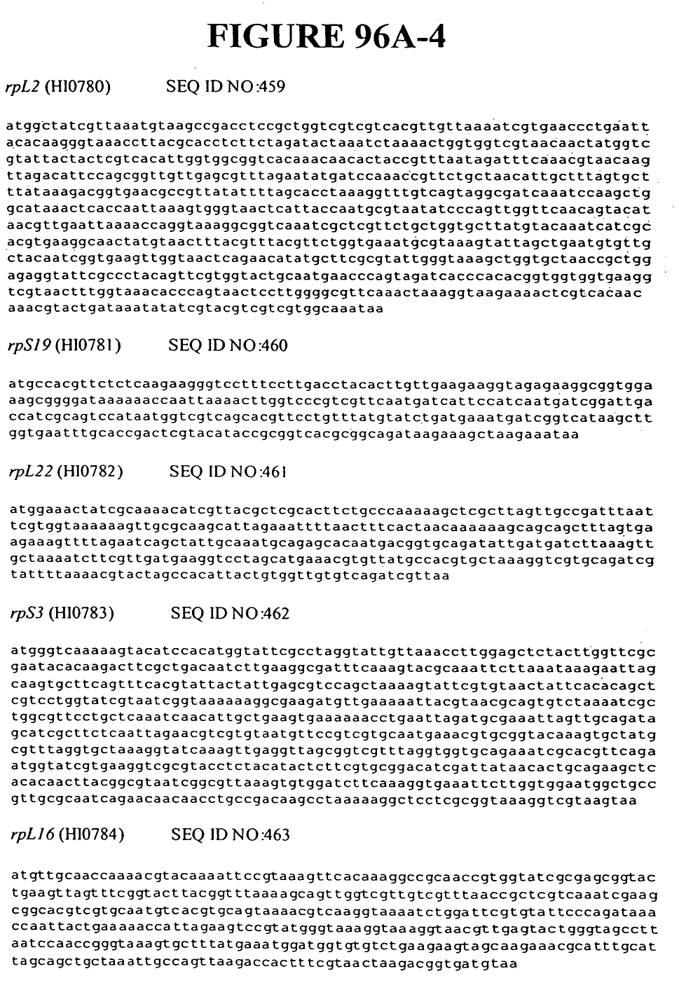

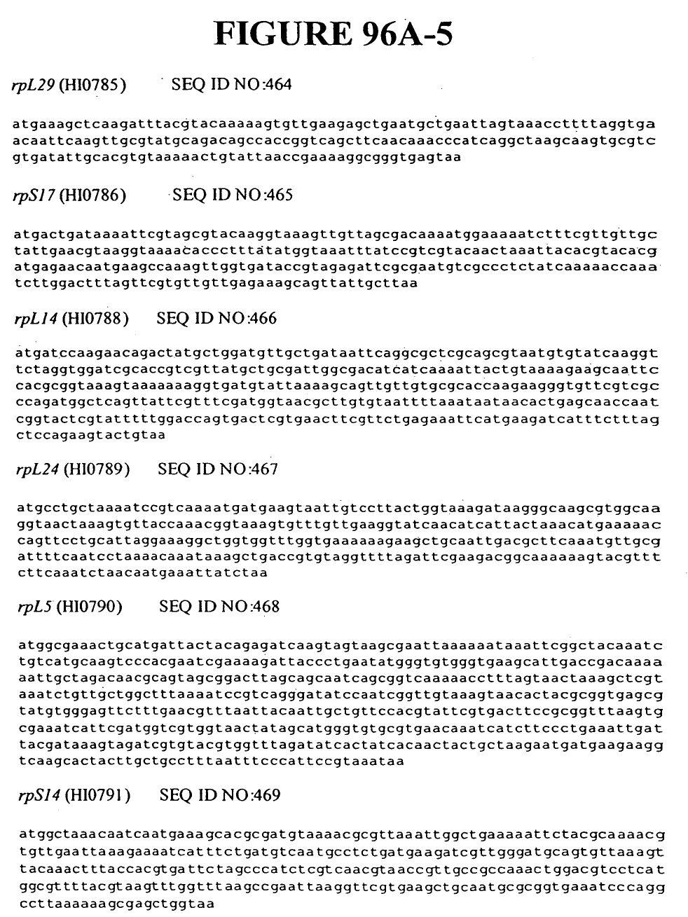

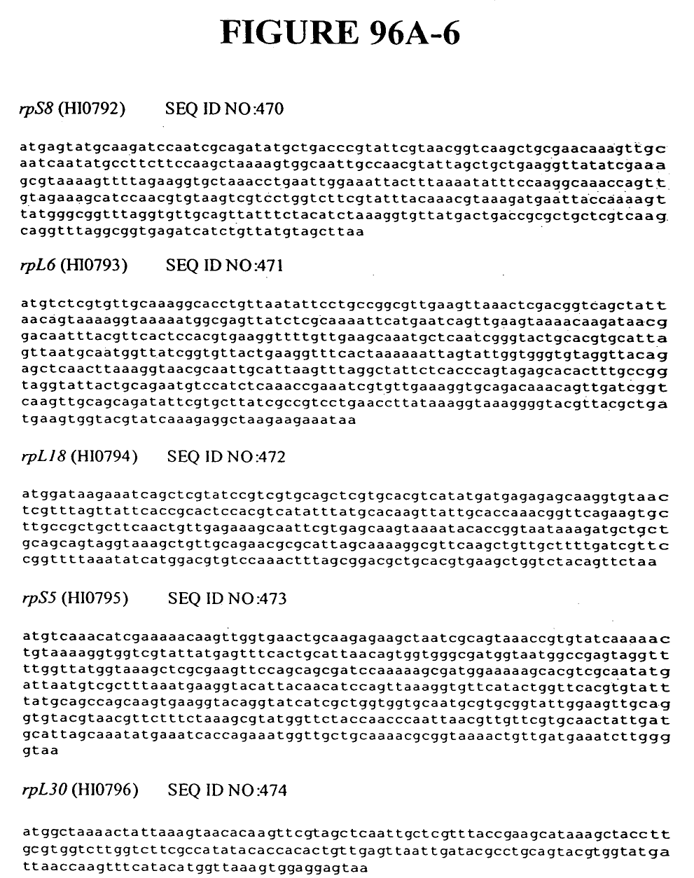

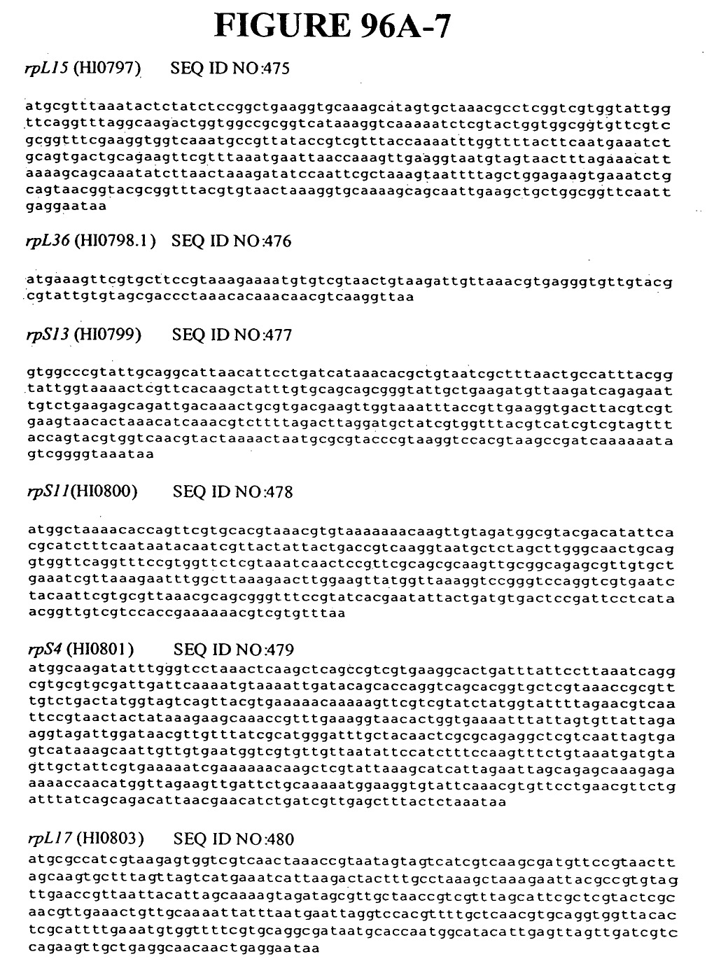

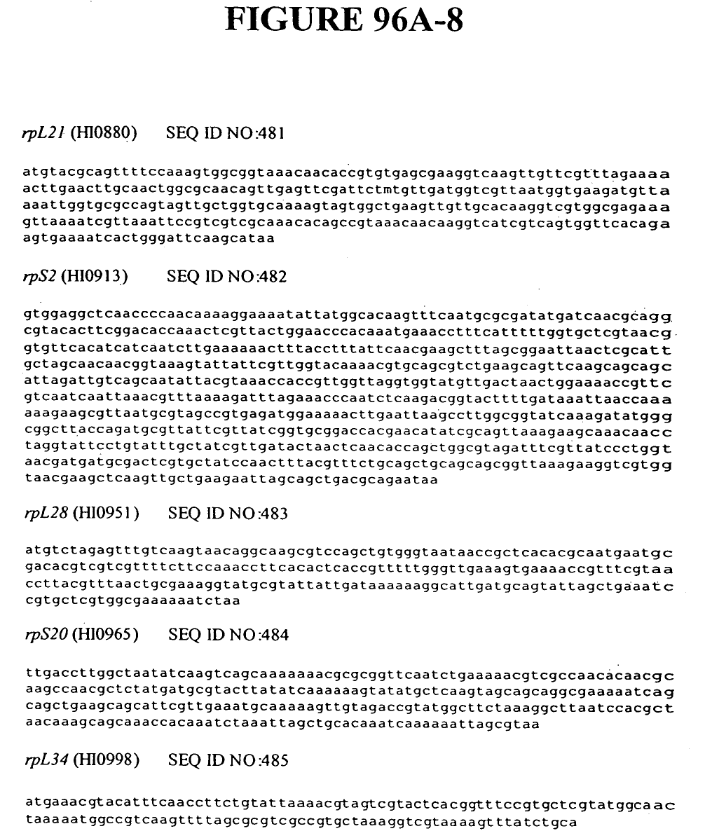

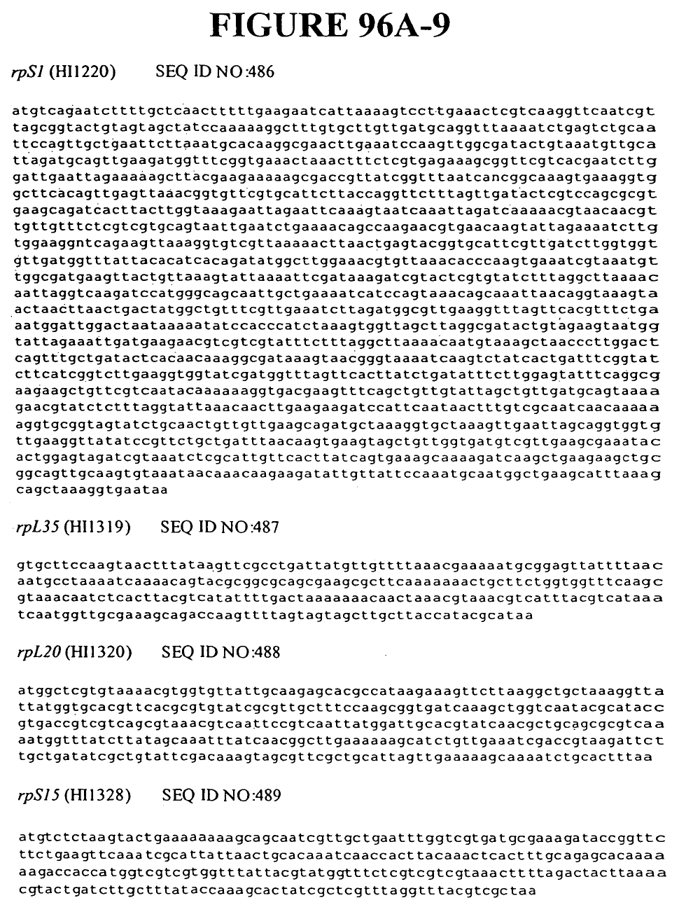

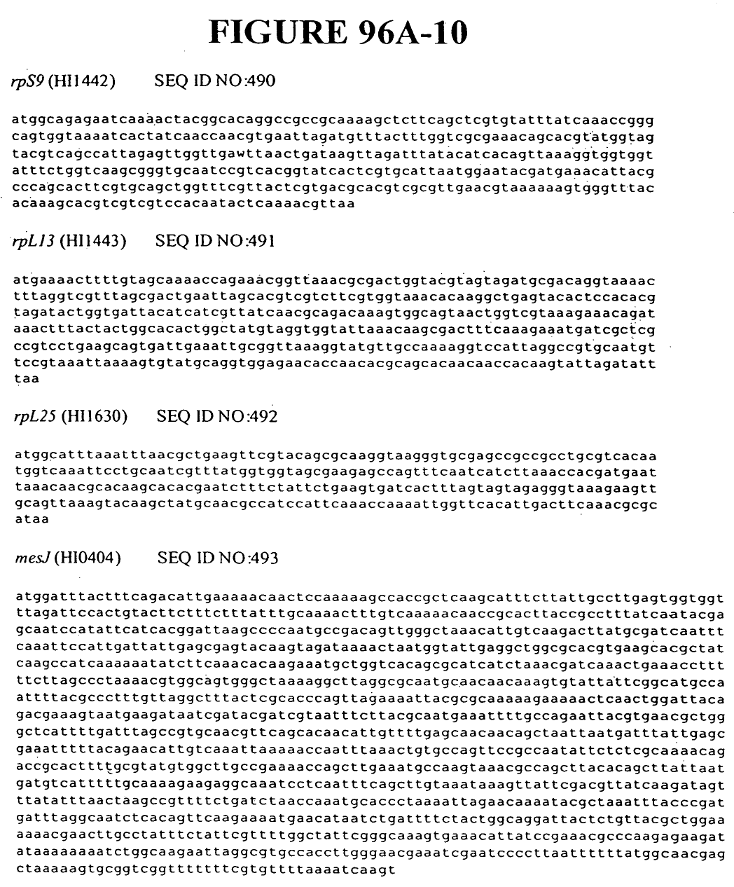

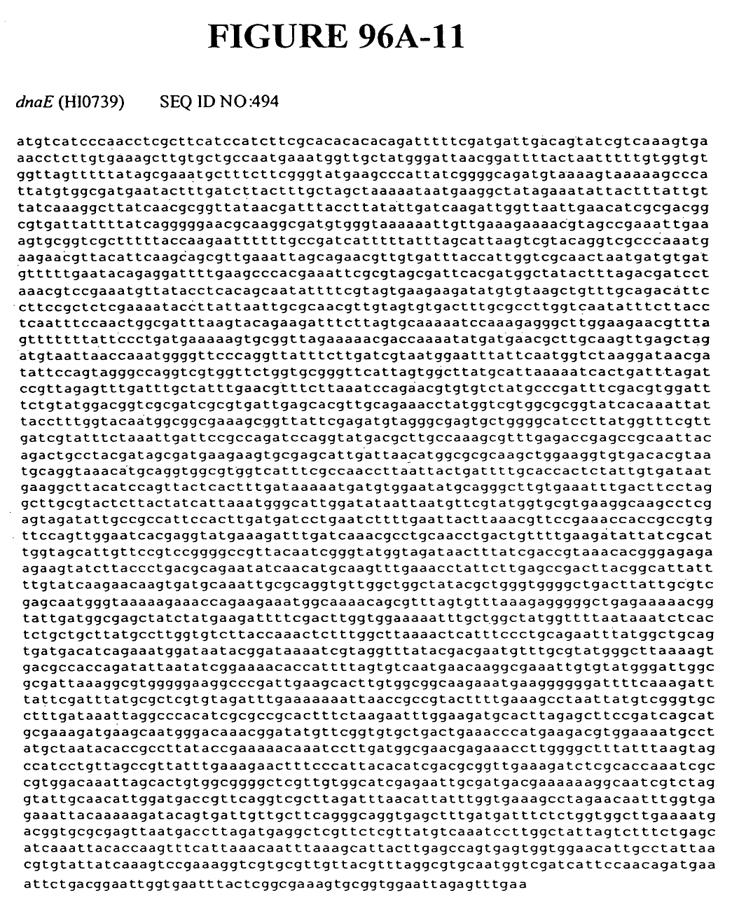

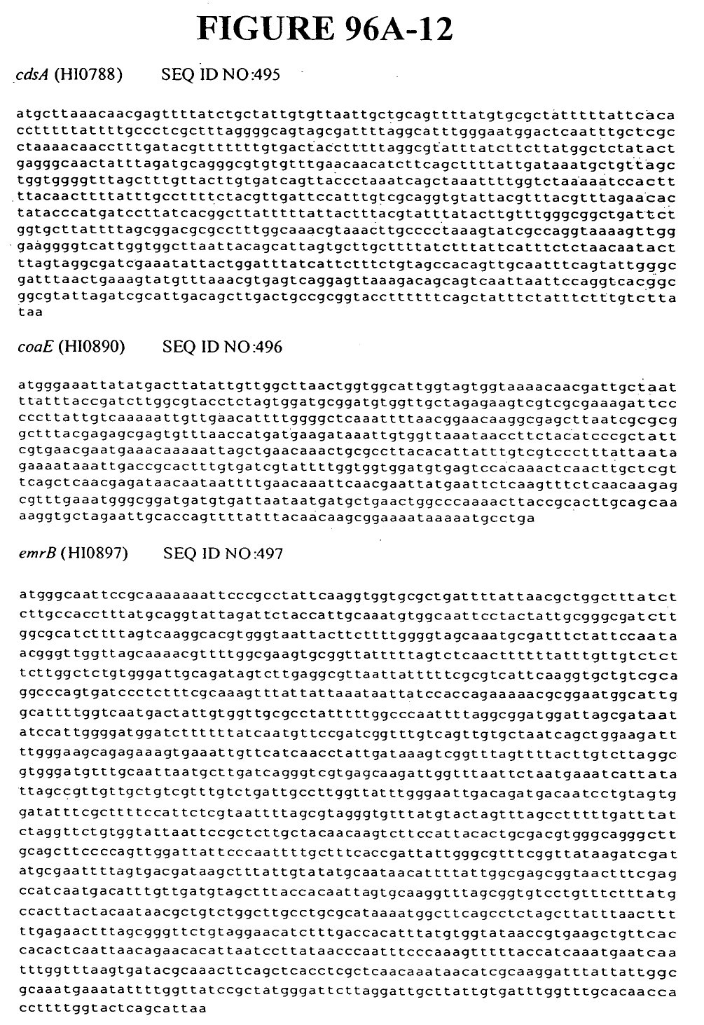

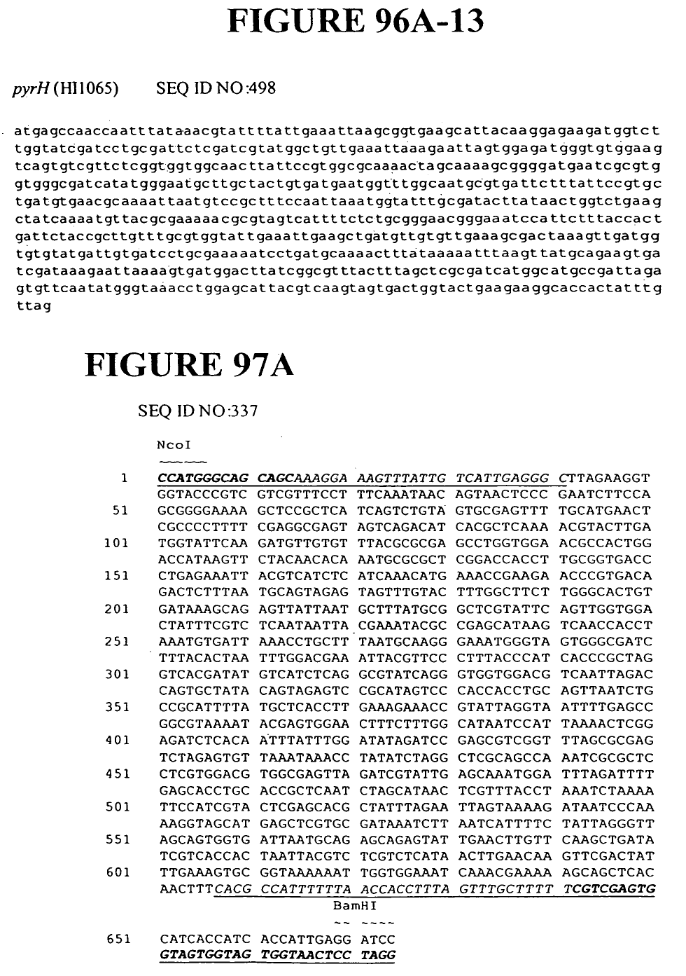

The following genes were subjected to insertional activation: dnaE (DNA polymerase III, alpha subunit), mesJ (cell cycle protein), cdsA (CDP-diglyceride synthetase), pyrH (uridylate kinase), coaA (dephosphocoenzyme A kinase) and emrB (multidrug resistance protein B). All of the gene sequences, flanking regions and PCR primer pairs are shown in FIG. 89-94. For each gene, the PCR product was synthesized, mutagenized with EZ::Tn<Tn>transposon, and transformed directly in H. influenzae. Kanamycin selected recombinants were screened with their essential region K/Z primer pair. The results are summarized in Table 10.

TABLE 10
Essentiality results for Example 6

| | | | SEQ ID NO: | | | |
|---|---|---|---|---|---|---|
| Gene | SEQ ID NO: | Locus | polypeptide | Function | Insertions | No. of inserts |
| dnaE | 494 | HI0739 | 439 | DNA polymerase III, alpha subunit | − | 0/96 |
| mesJ | 493 | HI0404 | 438 | Cell cycle protein | − | 0/96 |
| cdsA | 495 | HI0788 | 440 | CDP-diglyceride synthetase | − | 0/96 |
| pyrH | 498 | HI1065 | 443 | uridylate kinase | − | 0/96 |
| coaE | 496 | HI0890 | 441 | dephosphocoenzyme A kinase | − | 0/96 |
| emrB | 497 | HI0897 | 442 | multidrug resistance protein B | − | 0/96 |

### Example 7

### Cloning and Expression of an Essential Gene

Materials and Methods

Genomic Sequence and DNA. Sequence of H. influenzae imk was obtained from the GenBank database through the PubMed web interface, accession number NC_{—}000907. Genomic DNA from H. influenzae strain Rd (KW20) was obtained from American Type Culture Collection (ATCC 51907D).

PCR Protocol. PCR conditions were followed according to the Platinum PCR Enzyme System (GIBCO). 100 ng of genomic DNA was used with 200 nM primer concentration. Reactions were thermocycled in a GENEAMP PCR System 9700 (Perkin Elmer). Amplification was performed according to the following protocol: the reaction was incubated at 94° C. for 5 minutes, then cycled 30 times at 94° C. for 30 seconds (denaturation of DNA), 55° C. for 30 seconds (annealing of primers), and extension at 72° C. for 1 minute. The reaction finished with a final extension step performed at 72° C. for 30 minutes.

General Cloning Procedures. PCR products were ligated into pCR4-TOPO (Invitrogen) according to the manufacturer's protocol: 2-4 μL PCR product was mixed with 1 μL salt solution, 1 μL TOPO vector/enzyme solution, and 0-2 μL water to a final volume of 6 μL, and the reaction was incubated for 25-30 minutes at room temperature. Transformation was achieved by incubating 2 μL ligation mixture with 50 μL One Shot TOP10 cells on ice for 30 minutes, followed by heat shock at 42° C. for 45 seconds. After placing the cells on ice for 2 minutes, 250 μL SOC media was added, and the cells were incubated with shaking at 37° C. for 1 hour. Cells were spread on LB-Kanamycin (50 μg/mL) plates and incubated overnight at 37° C.

Overnight broth cultures (10 mL) of selected transformants underwent plasmid purification using the Qiagen QlAprep Spin Miniprep Kit. Plasmids exhibiting the proper restriction pattern were DNA sequenced to confirm nucleotide sequence of the insert.

The expression vector pET15b (Novagen) was digested with appropriate restriction enzymes and eletrophoresed in 0.8% SEAKEM GTG agarose (FMC Bioproducts). The appropriate bands were purified via the QIAEXII Gel Extraction Kit (Qiagen) and were ligated using T4 DNA ligase (Fermentas) and transformed via heat shock into TOP10 cells. Plasmids were purified and digested from selected atransformants to confirm proper ligation of insert into vector, and positive plasmid clones were transformed via heat shock into strains BL21 (DE3) and/or BL21 (DE3)pLysS (Novagen) according to manufacturer's instructions.

Cloning of Haemophilus influenzae tmk encoding a C-terminal hexahistidine tag. PCR was used to amplify tmk from Haemophilus influenzae strain Rd (KW20) genomic DNA (ATCC 51907D) as described in the general procedures above. Primers were designed to contain a 5′ NcoI and a 3′ BamHI site for cloning into the NcoI-BamHI sites of the expression vector pET15b. The 5′ primer used to clone H. influenzae tmk was also designed to encode a MGSS sequence at the N-terminus to improve efficiency of expression, while the 3′ primer was designed to encode a dual alanine linker segment followed by the hexahistidine encoding sequence (AAHHHHHH (residues 214-221 of SEQ ID NO:501)). Primer sequences were 5′-CCATGGGCAGCAGCAAAGGAAAGTTTA-TTGTCATTGAGGGC (N-terminal primer) (SEQ ID NO:502) and GGATCCTCAATGGTGATGGTGATGGTGAGCTGCTTTTTCGTTTGATTTCC A-CCAATTTTTTACCGCAC (C-terminal primer) (SEQ ID NO:503).

Expression. A single colony was picked from a fresh streak plate into NS86 seed medium containing ampicillin (100 μg/ml), grown to an absorbance of about 1 at 550 nanometers (˜1 A₅₅₀) at 30° C. and frozen ampules (20% glycerol was added as a cryoprotectant) prepared. Ampules were stored in the vapor phase of liquid nitrogen.

To prepare seeds, cells were grown in NS86 medium (2.6 g/l K₂HPO₄, 10.9 g/l NaNH₄HPO₄.4H₂O, 2.1 g/l citric acid, 0.67 g/l (NH₄)₂SO₄, 0.25 g/l MgSO₄.7H₂O, 10.4 g/l yeast extract and 5 g/l glycerol) containing ampicillin (100 μg/ml). Shake flask medium was MIM (32 g/l tryptone, 20 g/l yeast extract, 6 g/l Na₂HPO₄, 3 g/l KH₂PO₄, 0.5 g/l NaCl, and 1 g/l NH₄Cl) containing ampicillin (100 μg/ml).

Seeds were prepared by the inoculation of 0.1 ml thawed ampule-contents into 25 mls of NS86 medium and grown overnight at 30° C. Flasks (500 ml volume) containing 50 mls MIM medium were inoculated at 0.1 A₅₅₀. Cells were grown at 25° C. or 30° C. and induced at a density of ˜1 A₅₅₀ by the addition of IPTG.

SDS/PAGE analysis of culture samples was performed using the Laemmmeli procedure under reducing conditions. For Tmk titer estimation, 0.1 A-ml (where A-ml refers to A550 absorbance units per ml) cells pellets (lysed in sample buffer) were loaded; product was quantitated from the dried gels by densitometry using a Molecular Dynamics densitometer (Model 375A). Titer was estimated based on an external BSA standard (2 μg); specific expression level (% total cell protein, TCP) was calculated from the titer and the cell density using the conversion factors 1 A₅₅₀=0.26 g dry weight=0.14 g protein.

To assess soluble product, a 50 A-ml cell pellet was resuspended in 5 mls of cold PBS and the cells disrupted using a French Press. Following centrifugation (38, 700×g, 1 hour), a sample of the supernatant (soluble), as well as a sample of the unspun resuspension (total), were subjected to electrophoresis as described above. Densitometry on the dried gel provided a direct comparison of soluble to total product.

Results

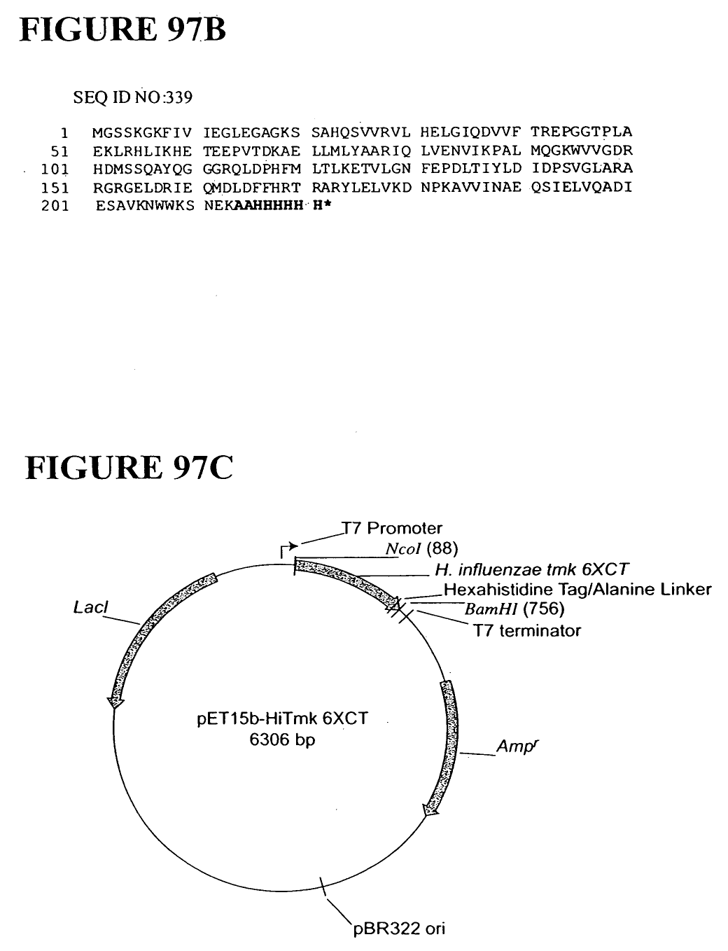

PCR amplification of tmk genes was performed using genomic DNA template as indicated in Materials and Methods. PCR products were ligated into pCR4-TOPO and transformed into E. coli TOP10. Recombinant plasmids were purified from selected transformants and DNA sequenced for confirmation. DNA sequencing results and the corresponding amino acid sequence is show in FIG. 97. The tmk gene of correct sequence was then ligated into the NcoI-BamHI sites of the expression vector pET15b (FIG. 97). Clones were transformed into E. coli BL21 (DE3) and frozen stocks were prepared.

Expression of H. influenzae Tmk was evaluated at both 25° C. and 30° C. and induction with 0.1 or 1 mM final concentration of IPTG. Both temperature and IPTG level had a dramatic effect on the expression as shown in Table 11. The ‘control’ conditions, 30° C. and 1 mM IPTG, resulted in a product titer of 288 mg/l (36% of total cell protein). When cells were induced with 0.1 mM IPTG (30° C.) the Tmk titer increased ˜2.5 times and reached 50% of the total cell protein. Dropping the temperature to 25° C. (0.1 mM IPTG resulted in titers that were ˜2 times that of the control (˜38% of total cell protein). Under all conditions, the Tmk was expressed exclusively in the soluble form.

TABLE 11
Effect of Temperature and IPTG Level on Tmk Expression

| Product | HiTmk 6XCT¹ | | |
|---|---|---|---|
| Temperature | 30° C. | 30° C. | 25° C. |
| IPTG (mM) | 1 | 0.1 | 0.1 |
| Titer (mg/l) | 288 | 749 | 528 |
| % TCP² | 36.6 | 50.4 | 38.0 |
| % Soluble | 100 | 100 | 100 |

¹HiTmk 6XCT; H. influenzae Tmk protein modified to encode a hexahistidine tag at the C-teminus of the protein.
²% of total cell protein

The complete disclosure of all patents, patent applications, and publications, and electronically available material (e.g., GenBank amino acid and nucleotide sequence submissions, and computer programs) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

1. An isolated polynucleotide comprising a nucleotide sequence having at least about 95 percent structural similarity with a nucleotide sequence selected from the group consisting of the coding sequence in SEQ ID NO:31, 99, 109, 124, 180, 210, 215, 200, and the complements thereof.

2. (canceled)

3. An isolated polynucleotide comprising a coding sequence encoding a polypeptide having at least about 95 percent structural similarity with an amino acid sequence selected from the group consisting of SEQ ID NO: 286, 301, 314, 322, 323, 327, and 299.

4. (canceled)

5. An isolated polypeptide comprising an amino acid sequence having at least about 95 percent structural similarity with an amino acid sequence selected from the group consisting of SEQ ID NO: 286, 299, 301, 304, 314, 322, 323, 327.

6. (canceled)

7. A method for identifying an agent that binds a polypeptide, the method comprising:
combining a polypeptide and an agent to form a mixture, wherein the polypeptide is encoded by a coding sequence comprising a nucleotide sequence having at least about 95 percent structural similarity with a nucleotide sequence selected from the group consisting of SEQ ID NO:31, 99, 109, 124, 180, 210, 215, 232, and 200; and
determining whether the agent binds the polypeptide.

8. The method of claim 7 wherein determining comprises an assay selected from the group consisting of an enzyme assay, a binding assay, and a ligand binding assay.

9. The method of claim 7 further comprising determining whether the agent decreases the growth rate of a microbe, comprising:
combining a microbe with the agent;
incubating the microbe and the agent under conditions suitable for growth of a microbe that is not combined with the agent; and
determining the growth rate of the microbe combined with the agent, wherein a decrease in growth rate compared to the microbe that is not combined with the agent indicates the agent decreases the growth rate of the microbe.

10. The method of claim 9 wherein the microbe is H. influenzae.

11-12. (canceled)

13. A method for identifying an agent that binds a polypeptide, the method comprising:
combining a polypeptide and an agent to form a mixture, wherein the polypeptide has at least about 95 percent structural similarity with an amino acid sequence selected from the group consisting of SEQ ID NO: 286, 299, 301, 304, 314, 318, 322, 323, and 327;
determining whether the agent binds the polypeptide.

14. The method of claim 13 wherein determining comprises an assay selected from the group consisting of an enzyme assay, a binding assay, and a ligand binding assay.

15. The method of claim 13 further comprising determining whether the agent decreases the growth rate of a microbe, comprising:
combining a microbe with the agent;
incubating the microbe and the agent under conditions suitable for growth of a microbe that is not combined with the agent; and
determining the growth rate of the microbe combined with the agent, wherein a decrease in growth rate compared to the microbe that is not combined with the agent indicates the agent decreases the growth rate of the microbe.

16. The method of claim 15 wherein the microbe is H. influenzae.

17. The method of claim 15 wherein the microbe is in vitro or in vivo.

18-36. (canceled)

37. A method for decreasing the growth rate of a microbe, the method comprising:
combining a microbe with an agent that binds to a polypeptide encoded by a coding sequence comprising a nucleotide sequence selected from the group consisting of SEQ ID NO: 31, 99, 109, 124, 180, 210, 215, 232, and 200.

38. The method of claim 37 wherein the microbe is in vitro or in vivo.

39-42. (canceled)

43. A method for making an H. influenzae with reduced virulence, the method comprising:
altering a coding sequence in an H. influenzae to comprise a mutation, the non-mutagenized coding sequence comprising a nucleotide sequence selected from the group consisting of SEQ ID NO:31, 99, 109, 124, 180, 210, 215, 232, and 200; and
determining if the H. influenzae comprising the mutation has reduced virulence compared to an H. influenzae that does not comprise the mutation.

44. The H. influenzae of claim 43 wherein the mutation is selected from the group consisting of a deletion mutation, an insertion mutation, a nonsense mutation, and a missense mutation.

45. An H. influenzae of claim 43.

46. A vaccine composition comprising the H. influenzae organism of claim 43.

47-54. (canceled)

55. The isolated polynucleotide of claim 1 wherein the nucleotide sequence is selected from the group consisting of the coding sequence in SEQ ID NO:31, 99, 109, 124, 180, 210, 215, 232, 200, and the complements thereof

56. The isolated polynucleotide of claim 3 comprising a coding sequence encoding a polypeptide having an amino acid sequence selected from the group consisting of 286, 299, 301, 304, 314, 318, 322, 323, and 327.
